(19) United States
(12) Patent Application Publication
(10) Pub. No.: US 2025/0120731 A1
(43) Pub. Date: Apr. 17, 2025

(54) OBSTRUCTION RETRIEVAL DEVICES

(71) Applicant: NeuRx Medical Inc., Philadelphia, PA (US)

(72) Inventor: Timothy William Ingraham Clark, Philadelphia, PA (US)

(21) Appl. No.: 18/488,283

(22) Filed: Oct. 17, 2023

(51) Int. Cl. A61B 17/221; A61M 25/00

(52) U.S. Cl. CPC A61B 17/221; A61B 2017/2212; A61M 25/0074

(57) ABSTRACT

Retrieval assemblies configured to capture an obstruction located in a bodily duct of a patient are provided. According to one implementation a retrieval assembly is provided that includes a distal collar operatively coupled to an elongate wire, a proximal collar slideable along a portion of the length of the elongate wire, and a plurality of circumferentially spaced-apart elongate clot capturing elements that each has a proximal end coupled to the proximal collar and a distal end coupled to the distal collar. The assembly being configured such that when the elongate wire is withdrawn proximally, the retrieval device transitions towards a fully expanded state.

### FIELD

The present invention relates to devices, assemblies, and methods for removing obstructions from a bodily duct of a patient, such as, for example, removing clots located within the vasculature of a patient.

### BACKGROUND

Existing clot retrieval devices are based on the concept of passing a guidewire through or near the center of the thrombus until the guidewire is beyond the thrombus, passing a delivery catheter over the guidewire across the thrombus and then exchanging the guidewire for a retriever. The delivery catheter is thereby withdrawn, exposing the retriever to the clot to enable capture after expansion of the retriever. This is followed by withdrawal of the clot-containing retriever device into an aspiration catheter positioned proximal to the clot of sufficient diameter to enable retrieval of the device with retention of the clot. One such retrieval device is the Solitaire™ revascularization device.

Most clot capturing elements of current retrievers are made of Nitinol and passively expand into and through the clot using the intrinsic radial force of the device prior to retraction of the device to remove the thrombus. This radial force is through thermally-induced, martensitic transformation of the Nitinol elements of the device by a predetermined shape change in the geometry of the Nitinol. By necessity, this expansion produces shearing and fracturing of the thrombus, since the clot capturing elements cannot expand through the clot to contact the vessel wall without producing linear defects within the clot. This clot shearing and fracturing can in turn increase the risk of distal embolization.

An estimated 700,000 patients in the U.S. and 950,000 in Europe suffer ischemic strokes each year, the majority of which are large vessel occlusions (LVOs). Some of the pivotal randomized prospective clinical trials to date have established the superiority of radially expandable retriever devices over drug therapy alone for LVOs of the anterior cerebral circulation. These clinical trials include: MR CLEAN, with published analysis appearing in the New England Journal of Medicine 2015; SWIFT PRIME, with published analysis appearing in the New England Journal of Medicine 2015; ESCAPE, with published analysis appearing in the New England Journal of Medicine 2015; EXTEND IA, with published analysis appearing in the New England Journal of Medicine 2015; REVASCAT, with published analysis appearing in the New England Journal of Medicine 2015 and THRACE, with published analysis appearing in Lancet Neurology 2016.

The published analyses of these trials have not emphasized the number of new ischemic strokes in the interventional arms of these studies compared to standard medical therapy. These new strokes are attributed to distal embolization, vessel spasm or dissection. In the MR CLEAN study, 5.6% (13 of 233) of patients in the retriever arm had clinical signs of a new ischemic stroke in a different vascular territory compared to 0.4% (1 of 267) in the control group, representing a 14-fold relative risk. In the REVASCAT study, 4.9% of patients had distal embolization into a new vascular territory compared to none in the control group, and 12.7% had local arterial complications attributed to passage of the retriever device (3.9% dissection, 4.9% perforation, and 3.9% vasospasm requiring treatment) compared to none in the control group. In the THRACE study, 6% had distal embolization in a new territory, and 26% experienced vasospasm, dissection, or perforation. None of these events occurred in the control group.

The number of additional passes (withdrawal of the retriever across the initial clot-containing segment of the vessel) has also been underemphasized in the clinical trials of these devices to date. Each pass of a retriever requires manipulation and advancement of a catheter and guidewire across the segment of the thrombus-containing artery to enable repeat delivery/deployment of the retriever, thereby creating potential vessel injury in the form of vasospasm, dissection or perforation. In a recent study, as many as 5 separate retriever passes were required for intended thrombus removal (REVIVE 2018) with a mean of 2.2 passes. The Instructions for Use (IFU) for the Solitaire™ thrombectomy device (sold by Medtronic) instructs physicians to perform no more than three recovery attempts in the same vessel. The IFU for the Trevo™ thrombectomy device (sold by Stryker) instructs physicians to exercise caution when withdrawing the device through an area or arterial vasospasm. The 3D Revascularization device (sold by Penumbra) enables up to 5 passes of the retriever device, each of which requires re-crossing of the target vessel (e.g. M1 segment of the middle cerebral artery) with a catheter and guidewire.

### SUMMARY

Obstruction retrieval devices, assemblies and methods are disclosed herein along with methods of manufacturing retrieval devices. Although the example implementations disclosed herein are directed to the removal of blood clots located within an artery of a patient, it is appreciated that the devices, assemblies and methods are applicable to the retrieval of other types of obstructions located in other bodily ducts of the patient.

According to some implementations a clot retrieval assembly is provided that includes an elongate wire having a proximal end, a distal end, and a longitudinal axis on which is mounted a retrieval device. According to some implementations the retrieval device includes a distal collar that is operatively coupled with the elongate wire. The retrieval device also includes a proximal collar having a longitudinal through opening through which the elongate wire passes. The proximal collar has a proximal facing surface. Disposed between the proximal and distal collars is a plurality of elongate clot capturing elements that each has a proximal end attached to the proximal collar and a distal end attached to the distal collar. According to some implementations, the proximal collar, distal collar, and elongate clot capturing elements are made from a single piece of material.

The retrieval device is configured to transition from an unexpanded state to an expanded state, wherein when in the unexpanded state no portion of the plurality of elongate clot capturing elements is located over the proximal collar and wherein when in the expanded state at least some portions of the plurality of elongate clot capturing elements are inverted and residing over the proximal collar. A tube is mounted on the elongate wire and has a longitudinal through opening through which the elongate wire passes. According to one implementation the proximal collar is attached to a distal end of the tube. According to an alternative implementation the proximal facing surface of the proximal collar faces a distal facing surface of the tube without there being an attachment of the proximal collar to the tube. In use, the retrieval device is configured to transition from the unexpanded state to the expanded state at least partially by withdrawing the elongate wire proximally while the tube is held stationary. The retrieval device, elongate wire and tube are assembled such that as the elongate wire is withdrawn proximally a distance between the proximal and distal collars decreases to induce a bending of the plurality of elongate clot capturing elements. According to some implementations the bending results in one or more or all of the plurality of elongate clot capturing elements assuming a looped configuration.

According to some implementations, the retrieval device is made of a shape memory alloy (e.g. Nitinol) and is configured to at least partially self-expand from the unexpanded state to a non-fully expanded state. This beneficially reduces the amount of axial force that needs to be applied to one or both of the proximal and distal collars when the retrieval device is initially transitioned away from the unexpanded state.

According to some implementations the retrieval device is delivered to the site of the clot in its radially unexpanded state inside a delivery catheter. According to some implementations, the retrieval device is deployed inside the duct (e.g. vasculature) of the patient at a location distal to the clot/obstruction. During deployment, the elongate clot capturing elements extend radially outward with some portions inverting around the proximal collar as explained above. After deployment of the retrieval device inside the duct of the patient to an expanded state, or a fully expanded state, the elongate wire is withdrawn proximally by the clinician to cause the elongate clot capturing elements of the retrieval device to engage the clot/obstruction.

According to some implementations, the clot/obstruction retrieval assembly is made by laser cutting a tubular member to produce the retrieval device that includes the proximal collar, the distal collar, and the plurality of elongate clot capturing elements that each has a proximal end attached to the proximal collar and a distal end coupled to the distal collar. The method also includes mounting the tube on the elongate wire, the tube having a longitudinal through opening through which the elongate wire passes.

The retrieval device is also mounted on the elongate wire at a location distal to the tube such that the elongate wire extends through the proximal collar and at least partially through the distal collar. According to one implementation the proximal collar is attached to a distal end of the tube. According to an alternative implementation the proximal facing surface of the proximal collar faces a distal facing surface of the tube without there being an attachment of the proximal collar to the tube. The distal collar is operatively coupled to a distal end or a distal end portion of the elongate wire. “Operatively coupled” meaning that 1) the distal collar is attached fixed to the elongate wire, or 2) a distal facing surface of the distal collar faces a proximal facing surface of a stop formed on or otherwise attached to the elongate wire without the distal collar being attached to the elongate wire. As noted above, the retrieval device, elongate wire and tube are assembled such that as the elongate wire is withdrawn proximally and the tube is held stationary a distance between the proximal and distal collars decreases to cause a bending and radial expansion of the plurality of elongate clot capturing elements.

According to other implementations, a clot retrieval assembly is provided that includes an elongate wire with first and second retrieval devices mounted thereon, with the first retrieval device located distal to the second retrieval device. Each of the first and second retrieval devices includes a distal collar, a proximal collar, and a plurality of elongate clot capturing elements that each has a proximal end attached to the proximal collar and a distal end attached to the distal collar.

Each of the first and second retrieval devices is configured to transition from an unexpanded state to an expanded state. In the unexpanded state no portion of the plurality of elongate clot capturing elements is located over the proximal collar of the respective first and second retrieval devices. However, in the expanded state at least a portion of the plurality of elongate clot capturing elements are inverted and residing over the proximal collar of the respective first and second retrieval devices. According to some implementations, the proximal collar, distal collar, and elongate clot capturing elements of each of the first and second retrieval devices are made from a single piece of material.

The clot retrieval assembly includes a first tube slidably mounted on the elongate wire between the proximal collar of the first retrieval device and the distal collar of the second retrieval device. According to some implementations the first tube has a distal end attached to the proximal collar of the first retrieval device and a second end attached to the distal collar of the second retrieval device. According to other implementations, the first tube is not attached to either of the first and second retrieval devices and has a distal facing surface that faces the proximal facing surface of the proximal collar of the first retrieval device, and has a proximal facing surface that faces the distal facing surface of the distal collar of the second retrieval device. Furthermore, according to some implementations, the first tube and the proximal collar, distal collar and elongate clot capturing elements of each of the first and second retrieval devices are all made from a single piece of material.

The clot retrieval assembly includes a second tube mounted on the elongate wire and has a longitudinal through opening through which the elongate wire passes. According to some implementations the proximal collar of the second retrieval device is attached to a distal end of the second tube. According to an alternative implementation the proximal facing surface of the proximal collar of the second retrieval device faces a distal facing surface of the second tube without there being an attachment of the proximal collar of the second retrieval device to the second tube.

The first and second retrieval devices are each configured to transition from their respective unexpanded states to their respective expanded states at least partially by withdrawing the elongate wire proximally while holding the second tube stationary. More particularly, the first and second retrieval devices, elongate wire and first and second tubes are assembled such that as the elongate wire is withdrawn proximally while the second tube is held stationary a distance between the proximal and distal collars of each of the first and second retrieval devices decreases. This induces a bending and radially expansion of the plurality of elongate clot capturing elements of each of the first and second retrieval devices. According to some implementations the bending and radial expansion of the elongate clot capturing elements results in the formation of a plurality of at least partially overlapping and radially extending petal-like/looped structures that are configured to engage the clot/obstruction.

According to some implementations, the elongate clot capturing elements of the first retrieval device have a width and/or a thickness that is different that the respective width and/or thickness of the elongate clot capturing elements of the second retrieval device. According to some implementations, the width and/or thickness dimensions of the elongate clot capturing elements are selected such that the elongate clot capturing elements of one of the first and second retrieval devices is stiffer than the elongate clot capturing elements of the other. According to one such implementation, the stiffness of the elongate clot capturing elements of the first retrieval device is greater than the stiffness of the elongate clot capturing elements of the second retrieval device. According to another implementation, the stiffness of the elongate clot capturing elements of the second retrieval device is greater than the stiffness of the elongate clot capturing elements of the first retrieval device.

According to some implementations, when the first and second retrieval devices are in their respective fully expanded state, one of the first and second retrieval devices has a fully expanded diameter greater than the fully expanded diameter of the other. According to one such implementation, the first retrieval device has a fully expanded diameter greater than that of the second retrieval device. A retrieval device is considered to be in a “fully expanded state” when a distance between the proximal and distal collars is at a minimum. Typically the distance between the proximal and distal collars is at a minimum when at least some of the proximal end portions of the elongate clot capturing elements make contact with or are pressed against at least some of the distal end portions of the elongate clot capturing elements as, for example, shown in FIG. 22B.

According to some implementations, the elongate clot capturing elements of the first and second retrieval devices differ in width and/or in thickness (which results in different stiffness characteristics) in addition to the first and second retrieval devices having different expanded diameters. For example, according to one implementation the elongate clot capturing elements of the second retrieval device are stiffer than those of the first retrieval device and the second retrieval device has a fully expanded diameter less than the fully expanded diameter of the first retrieval device.

According to some implementations, the clot/obstruction retrieval assembly is made by laser cutting a first tubular member to produce the first retrieval device and laser cutting a second tubular member to produce the second retrieval device. Each of the first and second tubular members is cut to produce in each retrieval device the proximal collar, the distal collar, and the plurality of elongate clot capturing elements that each has a proximal end attached to the proximal collar and a distal end coupled to the distal collar. The first and second retrieval devices are mounted on the elongate wire such that the elongate wire extends through their proximal and distal collars. The method also includes mounting the first and second tubes on the elongate wire such that the first tube resides between the proximal collar of the first retrieval device and the distal collar of the second retrieval device, and such that the second tube resides proximal to the second retrieval device. Each of the first and second tubes includes a longitudinal through opening through which the elongate wire passes. The first and second retrieval devices, first and second tubes, and elongate wire are assembled such that as the elongate wire is withdrawn proximally and the second tube is held stationary a distance between the proximal and distal collars in each of the first and second retrieval devise decreases to cause a bending and radial extension of the respective plurality of elongate clot capturing elements.

As noted above, according to some implementations the first tube and the proximal collar, distal collar and elongate clot capturing elements of each of the first and second retrieval devices are all made from a single piece of material.

According to some implementations the first and second retrieval devices are delivered to the site of the clot in their radially unexpanded state inside a delivery catheter. According to some implementations, the first and second retrieval devices are deployed inside the duct of the patient at a location distal to the clot/obstruction. During deployment of the retrieval devices, the clot capturing elements radially extend with some portions inverting around the proximal collar as explained above. After deployment of the retrieval devices inside the duct of the patient to their expanded state, or a fully expanded state, the elongate wire is withdrawn proximally by the clinician to cause the clot capturing elements of the second and first retrieval devices to sequentially engage the clot/obstruction.

According to other implementations, a clot retrieval assembly is provided that includes first, second and third retrieval devices mounted in series along a length of an elongate wire with the second retrieval device located between the first and third retrieval devices and the first retrieval device located distal to the third retrieval device. Each of the first, second and third retrieval devices includes a proximal collar, a distal collar and a plurality of elongate clot capturing elements that each has a proximal end attached to the proximal collar and a distal end attached to the distal collar. Each of the first, second and third retrieval devices is configured to transition from an unexpanded state to an expanded state. In the unexpanded state no portion of the plurality of elongate clot capturing elements is located over the proximal collar. In the expanded state at least some portions of the plurality of elongate clot capturing elements are inverted and reside over the proximal collar.

The clot retrieval assembly includes a first tube slidably mounted on the elongate wire between the proximal collar of the first retrieval device and the distal collar of the second retrieval device. According to some implementations the first tube has a distal end attached to the proximal collar of the first retrieval device and a second end attached to the distal collar of the second retrieval device. According to other implementations, the first tube is not attached to either of the first and second retrieval devices and has a distal facing surface that faces the proximal facing surface of the proximal collar of the first retrieval device, and has a proximal facing surface that faces the distal facing surface of the distal collar of the second retrieval device.

The clot retrieval assembly includes a second tube slidably mounted on the elongate wire that is located between the proximal collar of the second retrieval device and the distal collar of the third retrieval device. According to some implementations the second tube has a distal end attached to the proximal collar of the second retrieval device and a second end attached to the distal collar of the third retrieval device. According to other implementations, the second tube is not attached to either of the second and third retrieval devices and has a distal facing surface that faces the proximal facing surface of the proximal collar of the second retrieval device, and has a proximal facing surface that faces the distal facing surface of the distal collar of the third retrieval device.

The clot retrieval assembly also includes a third tube mounted on the elongate wire that has a longitudinal through opening through which the elongate wire passes. According to some implementations the proximal collar of the third retrieval device is attached to a distal end of the third tube. According to an alternative implementation the proximal facing surface of the proximal collar of the third retrieval device faces a distal facing surface of the third tube without the proximal collar of the third retrieval device being attached to the third tube.

The first, second and third retrieval devices are each configured to transition from their respective unexpanded states to their respective expanded states at least partially by withdrawing the elongate wire proximally while holding the second tube stationary. More particularly, the elongate wire, first, second and third retrieval devices and first, second and third tubes are assembled such that as the elongate wire is withdrawn proximally a distance between the proximal and distal collars of each of the first, second and third retrieval devices decreases. This induces a bending of the plurality of elongate clot capturing elements. According to some implementations the bending of the clot capturing elements results in each of the first, second and third retrieval devices the formation of a plurality of at least partially overlapping and radially extending petal-like/looped structures that are configured to engage the clot/obstruction.

According to some implementations, the elongate clot capturing elements of the first retrieval device have a width and/or a thickness that is different that the respective width and/or thickness of the elongate clot capturing elements of one or both of the second and third retrieval devices. According to some implementations, the width and/or thickness dimensions of the elongate clot capturing elements are selected such that the elongate clot capturing elements of at least one of the first, second and third retrieval devices is stiffer than the elongate clot capturing elements of one of the others. According to one such implementation, the stiffness of the elongate clot capturing elements of the third retrieval device is greater than the stiffness of the elongate clot capturing elements of the second retrieval device, and the stiffness of the elongate clot capturing elements of the second retrieval device is greater than the stiffness of the elongate clot capturing elements of the first retrieval device.

According to another implementation, the stiffness of the elongate clot capturing elements of the first retrieval device is greater than the stiffness of the elongate clot capturing elements of each of the second and third retrieval devices.

According to some implementations, when the first, second and third retrieval devices are in their respective fully expanded state, one of the first, second and third retrieval devices has an expanded diameter greater than the expanded diameter of at least one of the other retrieval devices. According to one such implementation, the first retrieval device has an expanded diameter greater than that one or both of the second and third retrieval devices. According to other implementations, the first retrieval device has an expanded diameter greater than the second retrieval device and the second retrieval device has an expanded diameter greater than that of the third retrieval device.

According to some implementations, the elongate clot capturing elements of the first, second and third retrieval devices differ in width and/or in thickness (which results in different stiffness characteristics) in addition to the first, second and third retrieval devices having different fully expanded diameters. For example, according to one implementation the elongate clot capturing elements of the third retrieval device are stiffer than those of the first retrieval device and the third retrieval device has an expanded diameter less than the expanded diameter of the first retrieval device.

According to some implementations the first, second and third retrieval devices are each delivered to the site of the clot/obstruction in its radially unexpanded state inside a delivery catheter. According to some implementations, the first, second and third retrieval devices are deployed inside the duct of the patient at a location distal to the clot/obstruction. During deployment of the retrieval devices, the clot capturing elements extend radially with portions thereof inverting around the proximal collar as explained above. After deployment of the retrieval devices inside the duct of the patient to an expanded state, or a fully expanded state, the elongate wire is withdrawn proximally by the clinician to cause the clot capturing elements of the third, second and first retrieval devices to sequentially engage the clot/obstruction.

These and other implementations along with their advantages and features will become evident in view of the drawings and the detailed description.

### BRIEF DESCRIPTION OF THE DRAWINGS

FIG. 1A illustrates an obstruction retrieval assembly according to one implementation with the retrieval device in an unexpanded state.

FIG. 1B illustrates the obstruction retrieval assembly of FIG. 1A with the retrieval device in a fully expanded state.

FIG. 1C is an enlarged view of the retrieval device depicted in FIG. 1A

FIG. 1D is an enlarged view of the retrieval device depicted in FIG. 1B.

FIG. 1E is a front view of the retrieval device depicted in FIG. 1D.

FIG. 2A shows an alternative implementation of FIG. 1A wherein a distal end of the distal collar of the retrieval device is not fixed to the elongate wire and abuts a stop on the elongate wire.

FIG. 2B illustrates the obstruction retrieval assembly of FIG. 2A with the retrieval device in a fully expanded state.

FIG. 3 shows an alternative implementation of the assembly of FIG. 1A wherein a proximal end of the proximal collar is not fixed to the tube and abuts a ferrule fixed to a distal end of the tube.

FIG. 4A shows a distal facing surface of the distal collar of the retrieval device according to one implementation.

FIG. 4B shows a proximal facing surface of a stop fixed to a distal end or distal end portion of the elongate wire according to one implementation.

FIG. 5A shows a proximal facing surface of the proximal collar of the retrieval device according to one implementation.

FIG. 5B shows a distal facing surface of the tube depicted in FIGS. 1A, 1B, 2A and 2B according to one implementation.

FIG. 5C shows a distal facing surface of the ferrule depicted in FIG. 3 according to one implementation.

FIG. 6 illustrates an actuation device according to one implementation.

FIG. 7 shows a retrieval device according to one implementation in which at least a portion of the retrieval device is self-expandable.

FIG. 8 shows the retrieval device of FIG. 1C that is cut along its axial length and laid flat on a surface.

FIG. 9A shows a retrieval device similar to that of FIG. 1C that is cut along its axial length and laid flat on a surface, wherein the elongate clot capturing elements have proximal and distal sections of different widths.

FIG. 9B shows a front view of the retrieval device of FIG. 9A in the expanded state.

FIG. 10A shows a retrieval device similar to that of FIG. 1C that is cut along its axial length and laid flat on a surface, wherein a central section of the elongate clot capturing elements have a reduced width.

FIG. 10B shows a front view of the retrieval device of FIG. 10A in the fully expanded state.

FIG. 11A shows a retrieval device similar to that of FIG. 1C that is cut along its axial length and laid flat on a surface, wherein neighboring the elongate clot capturing elements have different widths.

FIG. 11B shows a front view of the retrieval device of FIG. 11A in the fully expanded state.

FIG. 12 shows a tube from which a retrieval device may be made, the tube having a thinned wall section.

FIG. 13 shows the retrieval device of FIG. 1C that is cut along its axial length and laid flat on a surface, wherein the elongate clot capturing elements have proximal and distal sections of different wall thicknesses.

FIG. 14 shows the retrieval device of FIG. 1C that is cut along its axial length and laid flat on a surface, wherein the elongate clot capturing elements have a central section having a reduced wall thickness.

FIG. 15 shows a section of the tube of FIGS. 1A, 1B, 2A, 2B and 3 having a plurality of perforations that extend through the wall of tube and arranged to create areas of articulations to enhance the longitudinal flexibility of the tube.

FIG. 16A illustrates an obstruction retrieval assembly comprising first and second retrieval devices mounted in series on an elongate wire in their unexpanded state.

FIG. 16B illustrates the obstruction retrieval assembly of FIG. 16A with each of the first and second retrieval devices being in a fully expanded state.

FIG. 17 shows an alternative implementation of the assembly of FIG. 16A wherein a distal end of the distal collar of the second retrieval device is not fixed to the elongate wire and abuts a stop on the elongate wire.

FIG. 18A is related to FIG. 17 and shows a proximal facing surface of a stop on a distal end or distal end portion of the elongate wire according to one implementation.

FIG. 18B is related to FIGS. 16A-B and 17 and shows a distal facing surface of the distal collar of the second retrieval device according to one implementation.

FIG. 19A is related to FIGS. 16A-B and 17 and shows a proximal facing surface of the first tube according to one implementation.

FIG. 19B is related to FIGS. 16A-B and 17 and shows a distal facing surface of the distal collar of the first retrieval device according to one implementation.

FIG. 19C is related to FIGS. 16A-B and 17 and shows a distal facing surface of the first tube according to one implementation.

FIG. 19D is related to FIGS. 16A-B and 17 and shows a proximal facing surface of the proximal collar of the second retrieval device according to one implementation.

FIG. 20A is related to FIGS. 16A-B and 17 and shows a proximal facing surface of the distal collar of the first retrieval device according to one implementation.

FIG. 20B is related to FIGS. 16A-B and 17 and shows a distal facing surface of the second tube according to one implementation.

FIG. 21 illustrates an actuation device according to one implementation.

FIG. 22A shows a retrieval device that is self-expandable to a radially unconstrained state.

FIG. 22B shows the retrieval device of FIG. 22A being in a fully expanded state.

FIGS. 23A-B show an implementation in which the elongate clot capturing elements of the first and second retrieval devices have different widths.

FIGS. 24A-B show another implementation in which the elongate clot capturing elements of the first and second retrieval devices have different widths.

FIGS. 25A-B show the first and second retrieval devices having different fully expanded state diameters.

FIGS. 26A-B show the first and second retrieval devices having different fully expanded state diameters and with their elongate clot capturing elements having different widths.

FIG. 27 shows an implementation in which the stiffness characteristics of the elongate clot capturing element of the first and second retrieval devices vary in a way that results in the second retrieval device expanding before the first retrieval device.

FIG. 28 shows a scenario in which the elongate clot capturing elements of the first retrieval device are inverted backwards.

FIG. 29 shows an assembly similar to that of FIG. 16A, wherein the first tube has a shorter length that results in at least a portion of the elongate clot capturing elements of the second retrieval device contacting at least a portion of the elongate clot capturing elements of the first retrieval device when each of the first and second retrieval devices are in a fully expanded state.

FIG. 30 shows the first and second retrieval devices of FIG. 29 with each being in a fully expanded state.

FIG. 31 shows an implementation of FIG. 29 wherein the first and second retrieval devices and the first tube are unitarily formed, being made from a single piece of material.

FIG. 32 shows an assembly similar to that of FIG. 16A, wherein a distal end of the first retrieval device abuts a proximal end of second retrieval device.

FIG. 33 shows the first and second retrieval devices of FIG. 32.

FIGS. 34A and 34B show configurations of the first and second retrieval devices of FIGS. 29-33 according to one implementation.

FIGS. 35A and 35B show configurations of the first and second retrieval devices of FIGS. 29-33 according to another implementation.

FIGS. 36A and 36B show configurations of the first and second retrieval devices of FIGS. 29-33 according to another implementation.

FIGS. 37A and 37B depict a clot/obstruction retrieval device assembly that includes a reinforcement member having struts that are coupled to a midsection of the retrieval device for the purpose of inhibiting a reverse inversion of the elongate clot capturing elements as the retrieval device is advanced proximally across a clot/obstruction.

FIG. 38A illustrates an obstruction retrieval assembly comprising first, second and third retrieval devices mounted in series on an elongate wire in their unexpanded state.

FIG. 38B illustrates the obstruction retrieval assembly of FIG. 38A with each of the first, second and third retrieval devices being in an expanded state.

FIGS. 39A-C show front views of an implementation of the first, second and third retrieval devices of the assembly of FIGS. 38A-B in their fully expanded state.

FIGS. 40A-C show front views of an implementation of the first, second and third retrieval devices of the assembly of FIGS. 38A-B in their fully expanded state.

FIGS. 41A-C show front views of an implementation of the first, second and third retrieval devices of the assembly of FIGS. 38A-B in their fully expanded state.

FIGS. 42A-C show front views of an implementation of the first, second and third retrieval devices of the assembly of FIGS. 38A-B in their fully expanded state.

FIGS. 43A-C show front views of an implementation of the first, second and third retrieval devices of the assembly of FIGS. 38A-B in their fully expanded state.

FIGS. 44A-C show front views of an implementation of the first, second and third retrieval devices of the assembly of FIGS. 38A-B in their fully expanded state.

FIGS. 45A-C show front views of an implementation of the first, second and third retrieval devices of the assembly of FIGS. 38A-B in their fully expanded state.

FIG. 46 illustrates a similar construction to that of FIG. 31 in which the tube is substituted with a set of circumferentially spaced-apart struts.

FIG. 47A shows a retrieval device according to another implementation in which the elongate clot capturing elements are selectively tapered to induce a more symmetric expansion of the retrieval device.

FIG. 47B is a representation of the retrieval device of FIG. 47A showing a change of width of the elongate clot capturing elements along their length.

### DETAILED DESCRIPTION

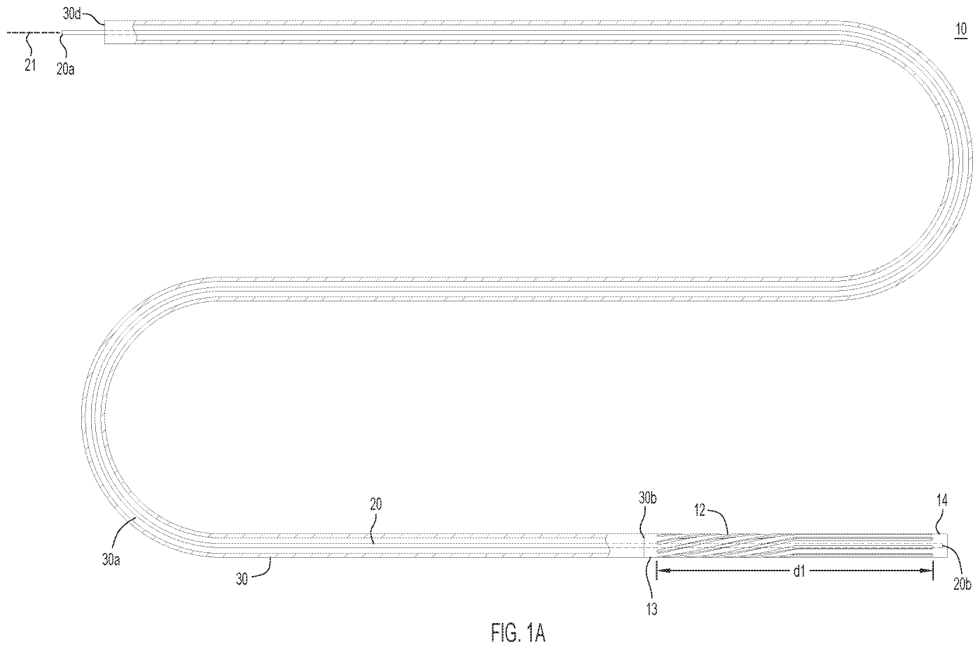

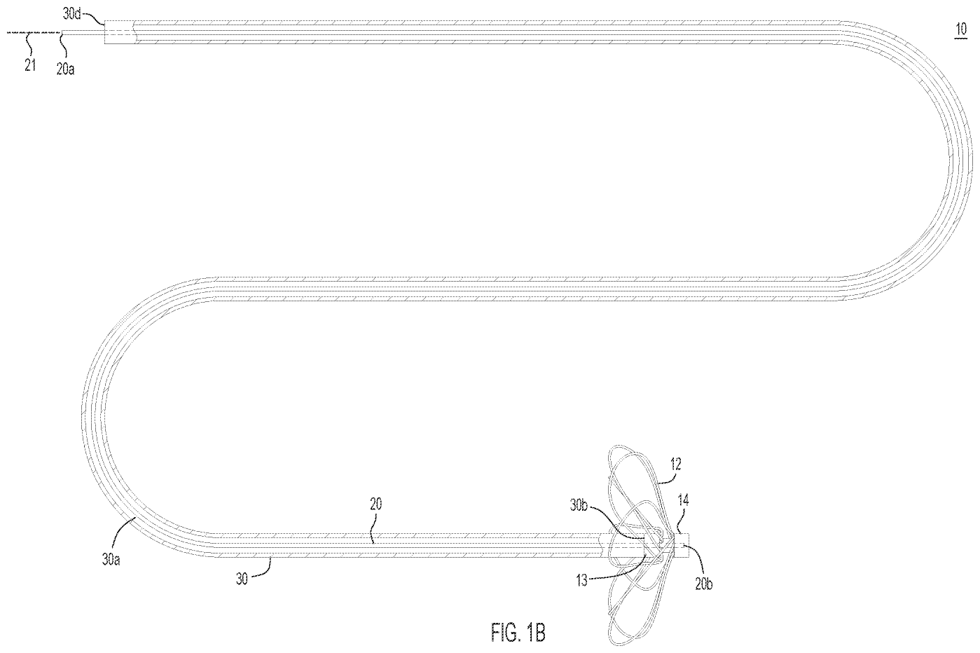

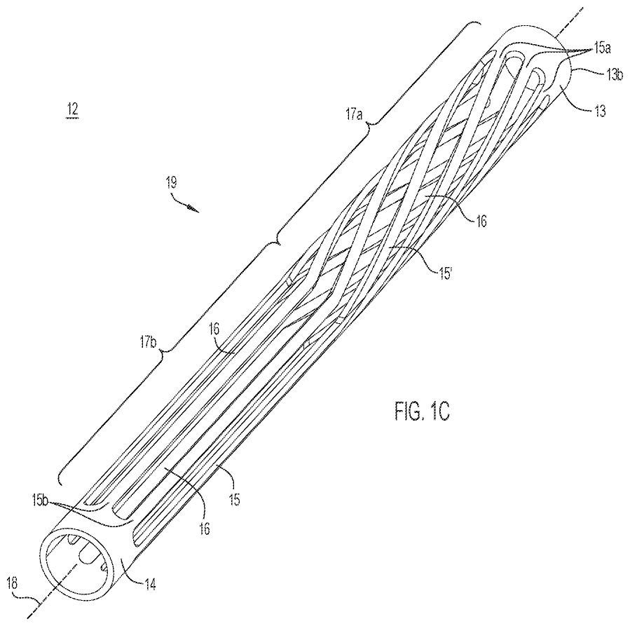

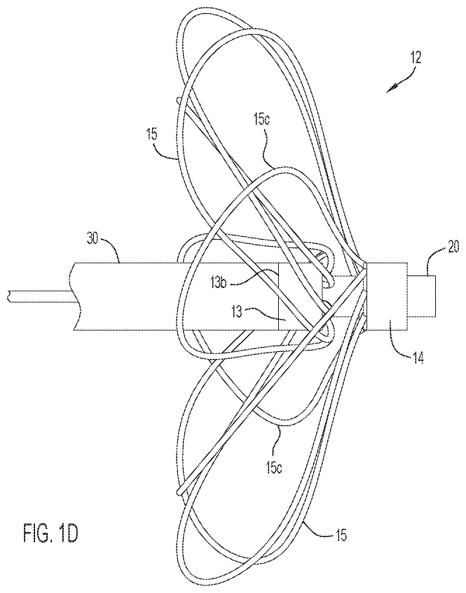

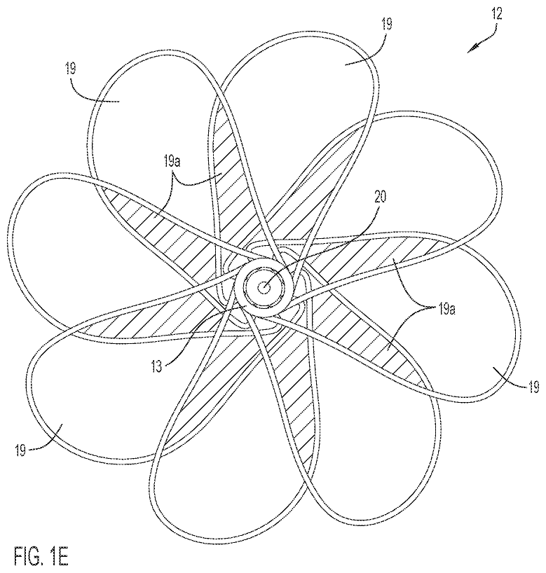

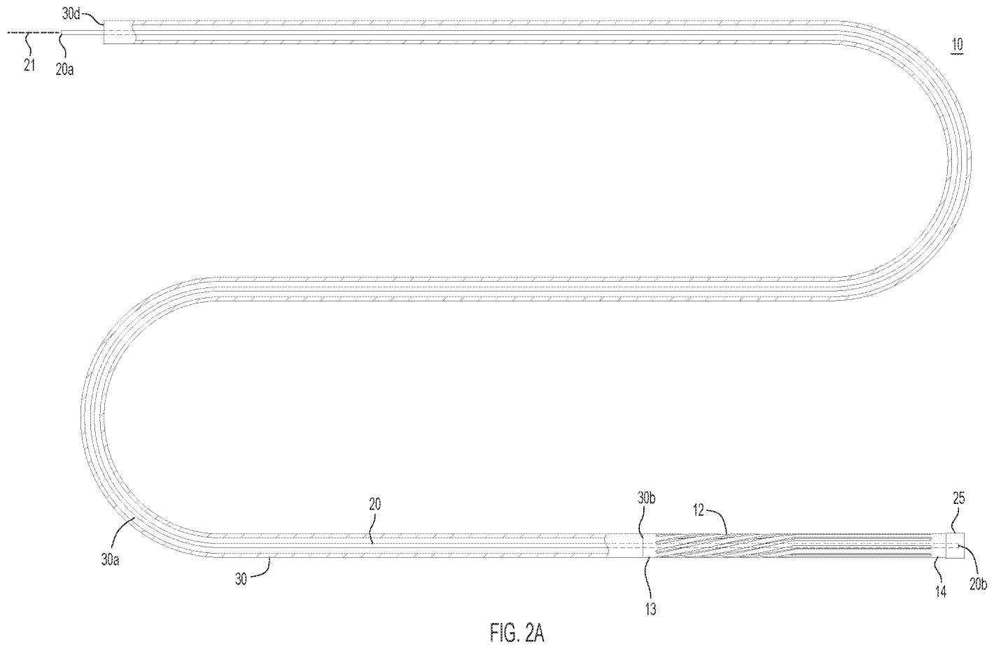

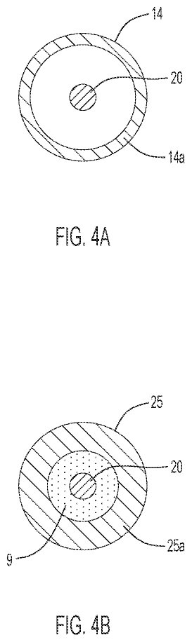

FIG. 1A illustrates an assembly 10 useful in removing obstructions (e.g. blood clots) located within a bodily duct of a patient. The assembly includes a retrieval device 12 that is transitional between an unexpanded state as shown in FIG. 1A and an expanded state as shown in FIG. 1B. The assembly includes an elongate wire 20 having a proximal end 20a, a distal end 20b, and a longitudinal axis 21 on which is mounted the retrieval device 12. The retrieval device 12 includes a distal collar 14 that is operatively coupled with the elongate wire 20. In the implementation of FIGS. 1A and 1B, the distal collar 14 is fixed to the distal end or distal end portion of the elongate wire 20. The distal collar 14 may be fixed to the elongate wire using an adhesive, by soldering, by welding, etc. In an alternative implementation, the distal collar 14 is not fixed to the elongate wire 20. In such implementations, as shown in FIGS. 2A and 2B, a stop 25 is provided on the elongate wire 20. The stop 25 includes a proximal facing surface 25a that faces a distal facing surface 14a of the distal collar 14 as shown in FIGS. 4A and 4B. The stop 25 may be formed integrally with the wire 20 or may be secured to the wire with the use of an adhesive 9 as shown in FIG. 4B, or by other fastening methods, such as soldering, welding, etc.

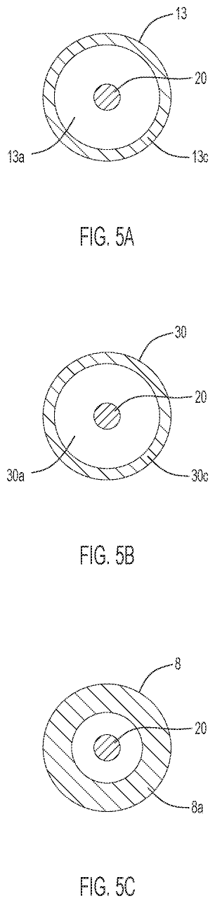

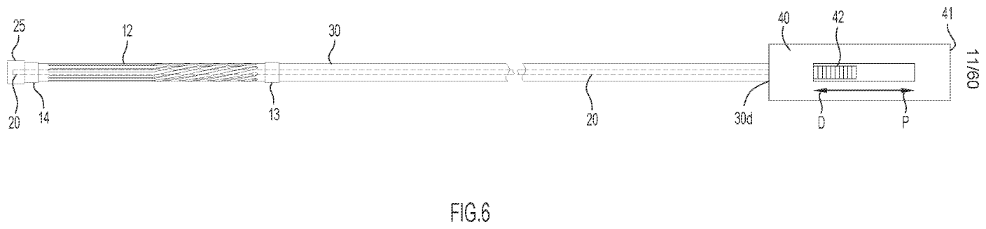

FIG. 1C illustrates an enlarged view of the retrieval device 12 in the unexpanded state and FIG. 1D illustrates an enlarged view of the retrieval device 12 in the expanded state. The retrieval device 12 also includes a proximal collar 13 having a longitudinal through opening 13a (FIG. 5A) through which the elongate wire 20 passes. Disposed between the proximal collar 13 and distal collar 14 is a plurality of elongate clot capturing elements 15 that each has a proximal end 15a attached to the proximal collar 13 and a distal end 15b attached to the distal collar 14. As shown in FIGS. 1A and 1C, when the retrieval device 12 is in the unexpanded state no portion of the plurality of elongate clot capturing elements 15 is located over the proximal collar 13. As shown in FIGS. 1B and 1D, when the retrieval device 12 is in the expanded state at least some portions 15c of the plurality of elongate clot capturing elements 15 are inverted and residing over the proximal collar 13. According to some implementations, at least some portions of the plurality of elongate clot capturing elements extend proximally to a location proximal to the proximal end 13b of the proximal collar 13 as shown in FIGS. 1B and 1D.

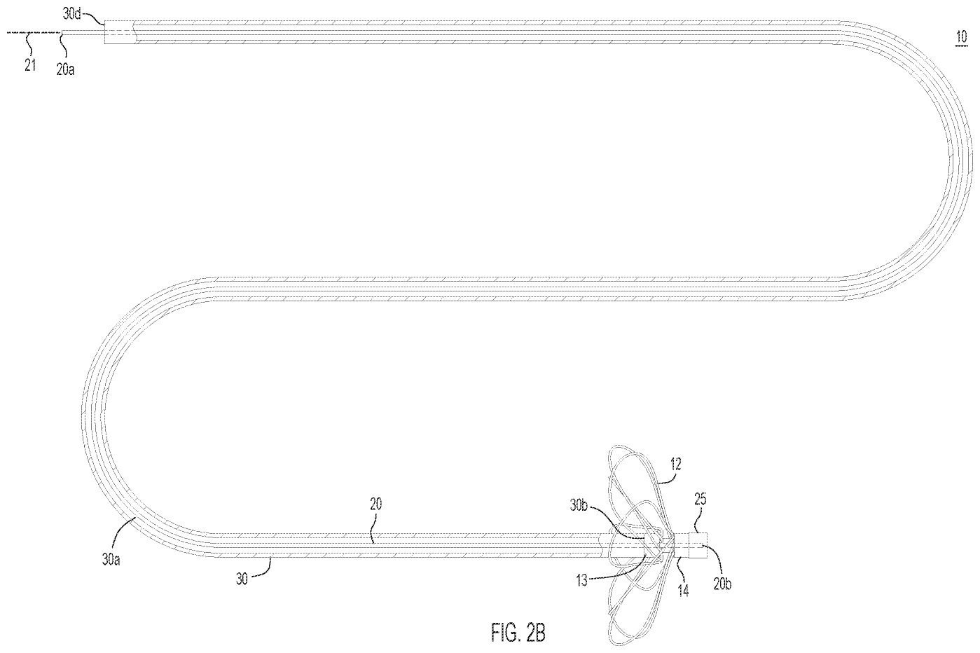

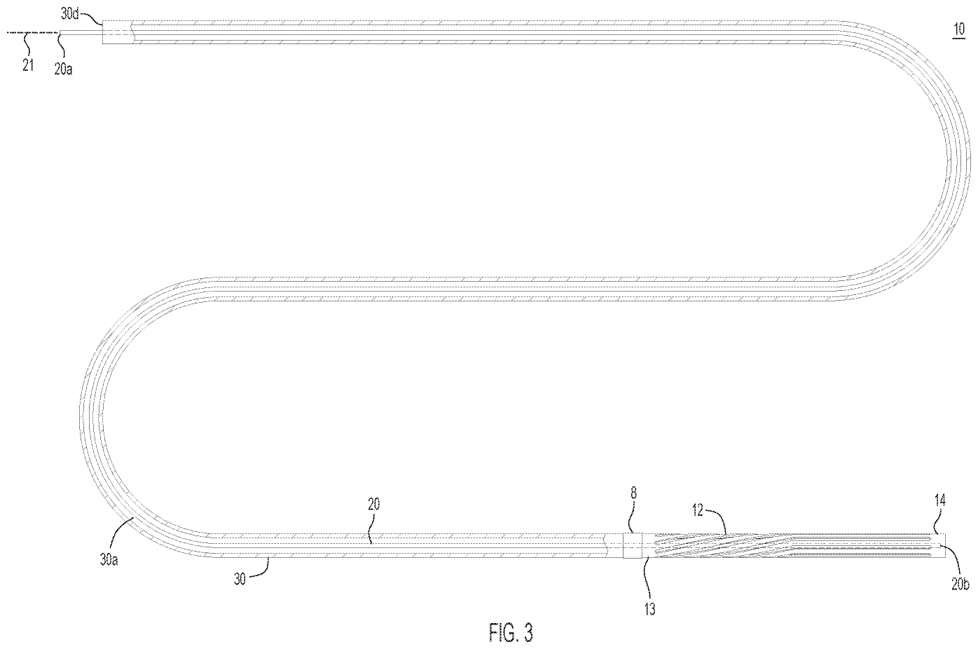

The assembly also includes a tube 30 mounted on the elongate wire 20. The tube 30 has a longitudinal through opening/passageway 30a through which the elongate wire 20 passes. According to some implementations, the proximal collar 13 is attached to a distal end 30b of the tube 30 by an adhesive, by soldering, welding, etc. In an alternative implementation, the proximal collar 13 is not fixed to the tube 30 and has a proximal facing surface 13c (FIG. 5A) that faces a distal facing surface 30c (FIG. 5B) of the tube 30. According to some implementations, as shown in FIGS. 3 and 5C, the distal end 30b of the tube 30 may be fitted with a ferrule 8 (or other hollow structure) that has a distal facing surface 8a that faces the proximal facing surface 13c of the proximal collar 13 of retrieval device 12. As shown in FIGS. 5B and 5C, the surface area of distal facing surface 8a of ferrule 8 is greater than the surface area of the distal facing surface 30c of tube 30.

According to some implementations, the assembly 10 is configured such that the retrieval device 12 transitions from the unexpanded state to the expanded state at least partially by a withdrawing of the elongate wire 20 proximally (in a direction towards the medical practitioner) while the tube 30 is held stationary. The retrieval device 12, elongate wire 20 and tube 30 are assembled such that as the elongate wire 20 is withdrawn proximally, the distal collar 14 moves proximally and a distance d1 (FIG. 1A) between the proximal collar 13 and distal collar 14 decreases to induce a bending of the plurality of elongate clot capturing elements 15 as shown in FIGS. 1B and 1D. According to some implementations, as shown in FIG. 1E, the elongate clot capturing elements 15 are configured to form petal-like structures 19 in which adjacent petal-like structures at least partially overlap with one another. The regions 19a of FIG. 1E identify such overlaps.

According to some implementations, the assembly 10 is also configured such that when the retrieval device 12 is in the expanded state, it is capable of transitioning from the expanded state towards or to the unexpanded state by a pushing of the elongate wire 20 distally (in a direction away from the medical practitioner). According to such implementations, the retrieval device 12, elongate wire 20 and tube 30 are assembled such that as the elongate wire 20 is pushed distally, the distance between the proximal collar 13 and distal collar 14 increases.

According to some implementations, the assembly 10 is configured such that retrieval device 12 transitions from the unexpanded state to the expanded state at least partially by a pushing of the tube 30 distally (in a direction away from the medical practitioner) while the elongate wire 20 is held stationary. According to such implementations, the retrieval device 12, elongate wire 20 and tube 30 are assembled such that as the tube 30 is pushed distally, the proximal collar 13 moves distally so that the axial distance d1 between the proximal collar 13 and distal collar 14 decreases to induce a bending of the plurality of elongate clot capturing elements 15.

According to some implementations, expansion of the retrieval device 12 occurs with the distal collar 14 fixed/attached to the elongate wire 20 and with the proximal collar 13 fixed/attached to tube 30. According to other implementations, expansion of the retrieval device 12 occurs with the distal collar 14 fixed to elongate wire 20 and with the proximal facing surface 13c of the proximal collar 13 abutting the distal facing surface 30c of the tube (surfaces 13c and 30c not being fixed/attached to one another). According to other implementations, expansion of the retrieval device 12 occurs with the proximal facing surface 25a of the stop 25 abutting the distal facing surface 14a of the distal collar 14 and with the proximal collar 13 fixed to tube 30 (surfaces 14a and 25a not being fixed/attached to one another). According to other implementations, expansion of the retrieval device 12 occurs with the proximal facing surface 25a of the stop 25 abutting the distal facing surface 14a of the distal collar 14 (surfaces 14a and 25a not being fixed/attached to one another) and with the proximal facing surface 13c of the proximal collar 13 abutting the distal facing surface 30c of the tube (surfaces 13c and 30c not being fixed/attached to one another).

To induce axial movement of the elongate wire 20 and/or the tube 30 to cause the retrieval device 12 to transition between the nonexpanded and expanded states may come in the form of a hand-held device that may be operated by the medical practitioner with the use of a single hand. FIG. 6 depicts an example actuation device 40 that includes an external housing 41 that is configured to be gripped by the hand of the medical practitioner. According to one implementation, the actuation device 40 includes a sliding member 42 to which is attached or coupled to the proximal end 20a or to a proximal end portion of the elongate wire 20. According to such an implementation, a proximal end 30d of tube 30 may be coupled or attached to a fixed part of the actuation device 40, such as a part of the housing 41. According to some implementations, the actuation device 40 is configured such that the sliding member 42 is capable of moving proximally in the P direction and distally in the D direction as shown in FIG. 6. According to one implementation, the starting position of the sliding member 42 is the forward-most position as shown in FIG. 6 and this position corresponds to the retrieval device 12 being in the unexpanded state. In use, upon the retrieval device 12 having been delivered to a location distal to an obstruction in the duct of the patient, the medical practitioner may act on the sliding member 42 with his/her thumb to move the sliding member proximally. This causes a withdrawing of the elongate wire 20 proximally, which by virtue of the distal collar 14 being operatively coupled with the elongate wire 20, results in the distal collar 14 advancing proximally nearer the proximal collar 13. As explained above, this results in a bending of the elongate clot capturing elements 15 so that they assume a looped or petal-like configuration sufficient to ensnare an obstruction. According to some implementations, the actuation device 40 is configured such that when the sliding member 42 is in the proximal-most position, the retrieval device 12 is in a fully expanded state.

According to some implementations, it may be beneficial to cause the retrieval device 12 to assume a partial expanded state or a fully unexpanded state after an obstruction has been captured. In such cases, the sliding member 42 may be advanced distally to cause the retrieval device 12 to assume the partial expanded state or unexpanded state.

As noted above, according to some implementations the retrieval device 12 is expanded by holding the elongate wire 20 stationary while advancing the tube 30 distally. This results in the distal facing surface 30c of tube 30 pushing against the proximal facing surface 13c of the proximal collar 13 of the retrieval device 12 to cause the proximal collar 13 to advance distally towards the distal collar 14. According to such implementations, the proximal end 20a or a proximal end portion of the elongate wire 20 is fixed to a stationary part of the actuation device 40 and the proximal end of the tube 30 is attached to or otherwise coupled to the sliding member 42.

According to other implementations, the actuation device 40 may be configured to cause an axial movement of both the elongate wire 20 and tube 30 in opposite directions to cause the retrieval device 12 to transition to the expanded state.

According to other implementations, a two-handed approach may be used to cause the retrieval device 12 to transition between the unexpanded and expanded states. This may be accomplished, for example, by holding one of the elongate wire 20 and tube 30 stationary while applying axial movement to the other.

According to some implementations, the proximal collar 13, distal collar 14 and plurality of elongate clot capturing elements 15 of the retrieval device 12 are made from a single piece of material. According to some implementations the retrieval device 12 is formed from a metal tubular member 19 as shown in FIG. 1C that is laser cut to form the plurality of elongate clot capturing elements 15. The metal tubular member 19 may be made of any of a variety of metals or metal alloys, such as, for example, stainless steel and Nitinol.

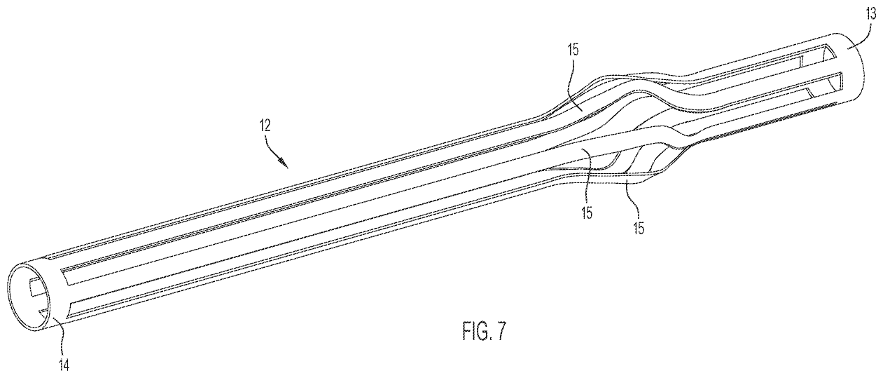

The retrieval device 12 may be constructed to transition from the unexpanded state to the expanded state only by applying axially directed forces to one or both of the proximal and distal collars 13 and 14 as discussed above. The retrieval device 12 may also be constructed to self-expand to at least a partially expanded state. According such implementations, a sheath may be used to constrain the clot capturing elements 15 prior to deploying the retrieval device 12 inside the duct of the patient. At the time of deployment, the sheath is removed and an axial force is applied to at least one of the proximal and distal collars 13 and 14 to cause the retrieval device to assume a partially or fully expanded state. FIG. 7 shows an example retrieval device 12 in a partially expanded state. A benefit of having the retrieval device 12 at least partially self-expand, is that it reduces the initial amount of axial force needed to be applied to one or both of the distal and proximal collars 13 and 14 during an initial expansion of the clot capturing elements 15. This makes it easier for the medical practitioner to deploy the retrieval device 12 and also reduces the risk of breakage. For example, because the initial amount of axial force needed to be applied is reduced, it is less likely the elongate wire 20 will decouple from the distal collar 14 or stop 25 as it is withdrawn proximally to move the distal collar 14 towards the proximal collar 13.

Shaping of the elongate clot capturing elements 15 to cause the retrieval device 12 to at least partially self-expand may include using a fixture to constrain the clot capturing elements 15 (made of a shape memory alloy) in a partially expanded state as shown, for example, in FIG. 7. Once constrained in the fixture, the clot capturing elements 15 may be heat treated to lock them in their partially expanded configurations. The degree to which the clot capturing elements 15 partially self-expand can vary. In the example of FIG. 7, the retrieval device 12 is shown in a rest state with the elongate clot capturing elements 15 not being radially constrained.

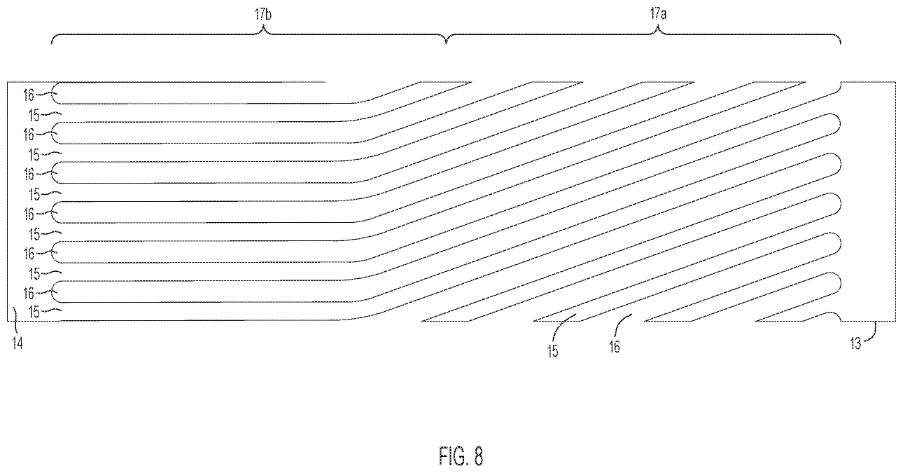

FIG. 1C shows a retrieval device 12 in which the elongate clot capturing elements 15 are formed by cutting slots 16 in a tubular member. Methods other than laser cutting a tube may also be employed to construct the retrieval device 12. Other such methods are disclosed in U.S. Pat. No. 10,555,745, issued Feb. 11, 2020, which is incorporated by reference herein in its entirety. Furthermore, the retrieval devices disclosed and contemplated herein may further be constructed to comprise elongate clot capturing elements shaped and oriented like those disclosed in U.S. Pat. No. 10,555,745. These include, in an unlimited way, the examples of FIGS. 8A-B, 8E, 9B, 10A-B, 12, 13A-B, 14A-B, 15A-C, 16A-B, 17A-B and 18A-B of U.S. Pat. No. 10,555,745.

In the example retrieval device 12 illustrated in FIGS. 1A and 1C, the proximal end sections 17a of the elongate clot capturing elements 15 are arranged in a helical pattern about the longitudinal axis 18 of the retrieval device and the distal end sections 17b of the elongate clot capturing elements 15 are arranged straight and parallel to the longitudinal axis 18. FIG. 8 shows the retrieval device of FIG. 1C cut along its axial length and laid flat on a surface.

By virtue of the helical nature of the proximal end section 17a of the clot capturing elements 15, as the distal and proximal collars 14 and 13 are brought closer together by the application of the proximal and/or distal forces discussed above the elongate clot capturing elements assume the looped or petal-like configuration as explained above. As the distal force and/or proximal force continues to be applied to the proximal collar 13 and/or distal collar 14, an inversion of the clot capturing elements 15 occurs as shown in FIG. 1D. The inversion results in at least a portion of one or more or all of the clot capturing elements 15 residing at least partially over the proximal collar 13 and/or at least partially surrounding the proximal collar. As the distance between the proximal and distal collars decreases, the clot capturing elements 15 continue to bend to produce, for example, looped elements like those shown in FIG. 1D with adjacent loops preferably overlapping one another.

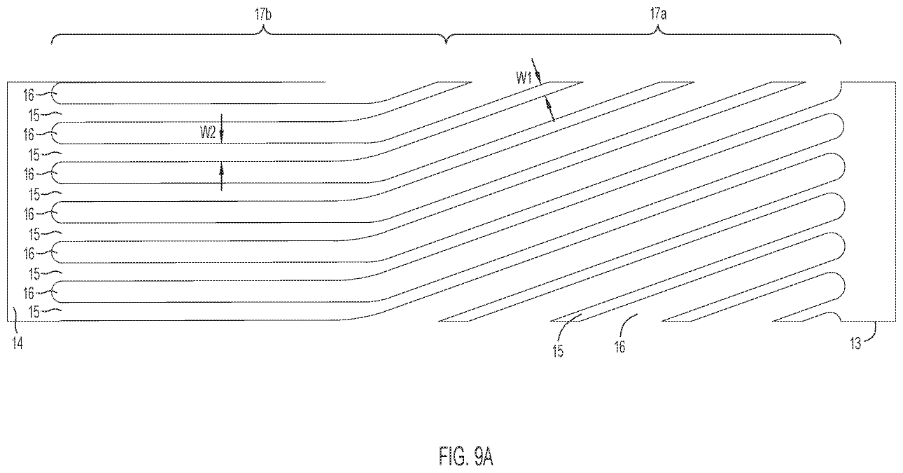

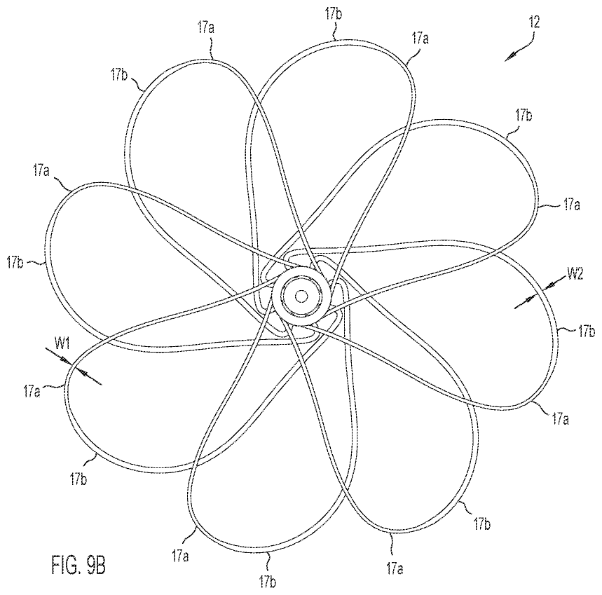

Although the width of the elongate clot capturing elements 15 may be constant/uniform along their length as shown in FIGS. 1C and 8, according to the implementation shown in FIGS. 9A and 9B, the proximal end sections 17a of the elongate clot capturing elements 15 have a width w1 that is less than the width w2 of the distal end sections 17b. The use of the wider distal struts assists in entrapping the clot/obstruction and blocking it from passing downstream the retrieval device. The wider distal struts also provide greater resistance to bending back on themselves as the device is withdrawn through the bodily duct of the patient. This lessens the likelihood of the retrieval device inverting backwards (like an umbrella in a strong wind) and losing an entrapped clot/obstruction. Because the proximal end sections of the clot capturing elements 15 have a smaller width w1 (as compared to w2), the axial force needed to be applied to the proximal and/or distal collars 13 and 14 to cause the retrieval device 12 to initially transition from an unexpanded to an expanded state is lessened.

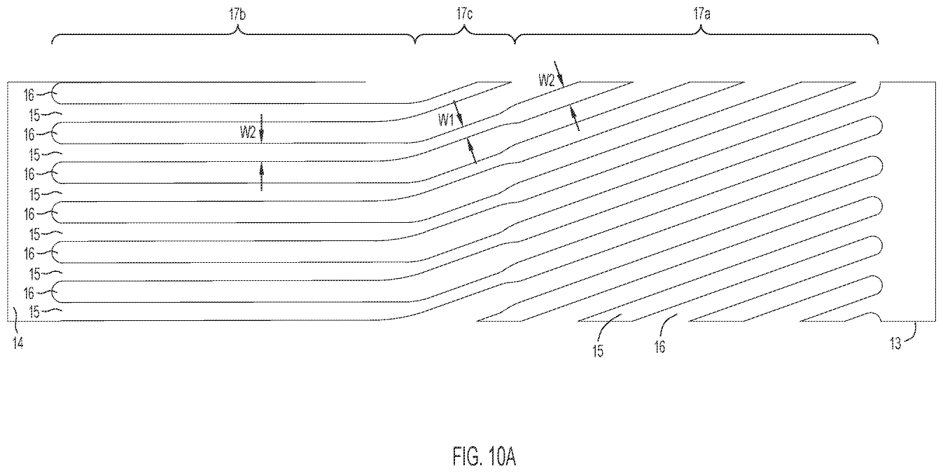

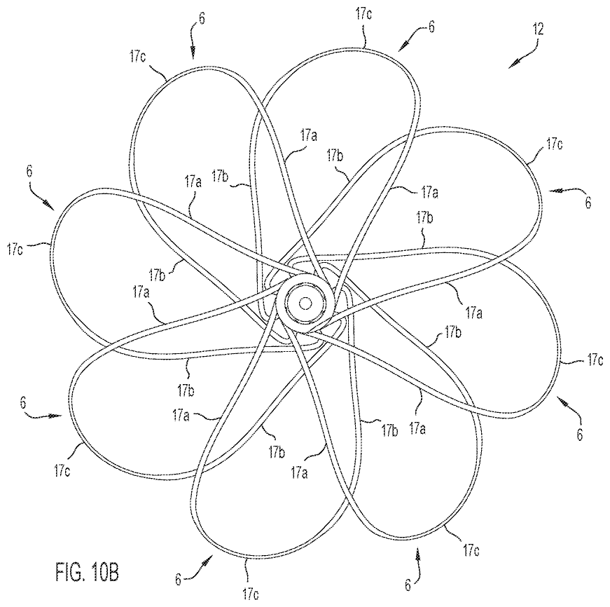

According to other implementations, like those shown in FIGS. 10a and 10B, the elongate clot capturing elements 15 may comprise a proximal section 17a, a distal section 17b and a central section 17c, wherein which the central section has a width w1 that is less than the widths w2 of the proximal and distal sections. As shown in FIG. 10B, because both the proximal and distal sections of the elongate clot capturing elements have a greater width, entrapment of firmer consistency or chronic clot/obstruction is enhanced in comparison to the retrieval device of FIG. 9B. Because the central section of the clot capturing elements 15 has a smaller width w1 (as compared to w2 of the proximal and distal end sections 17a and 17b), the apex region 6 of the petal-like structures formed when the retrieval device assumes its expanded state are more flexible than the proximal and distal end sections 17a and 17b. The more flexible central section 17c of the elongate clot capturing elements more gently contact the interior wall of the duct under treatment as the retrieval device is withdrawn therethrough. This can result in better therapeutic outcomes due to less vessel damage occurring during treatment.

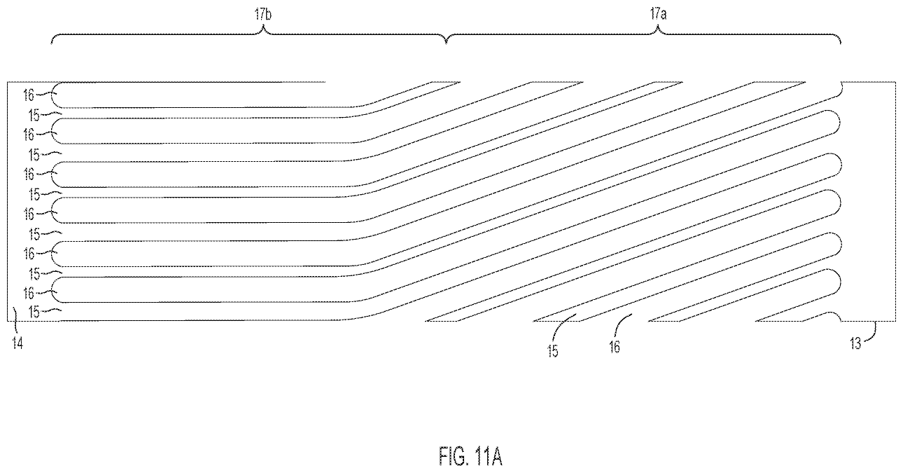

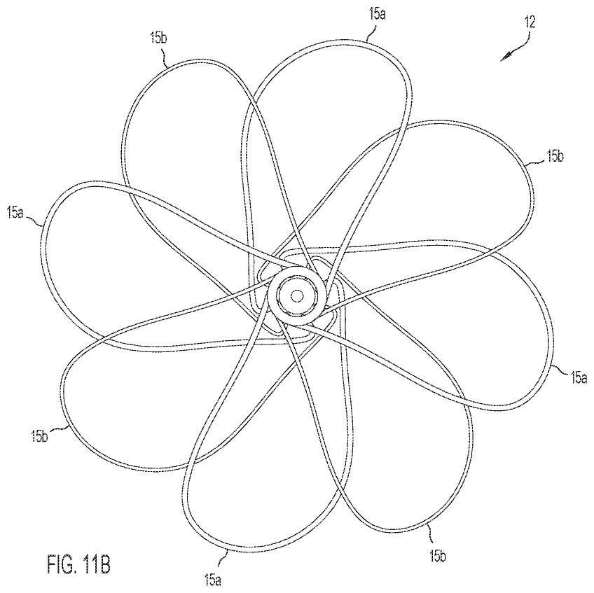

FIG. 11A shows a retrieval device similar to that of FIG. 1C that is cut along its axial length and laid flat on a surface, wherein neighboring the elongate clot capturing elements 15a and 15b have different widths w1 and w2. FIG. 11B shows a front view of the retrieval device of FIG. 11A in the expanded state.

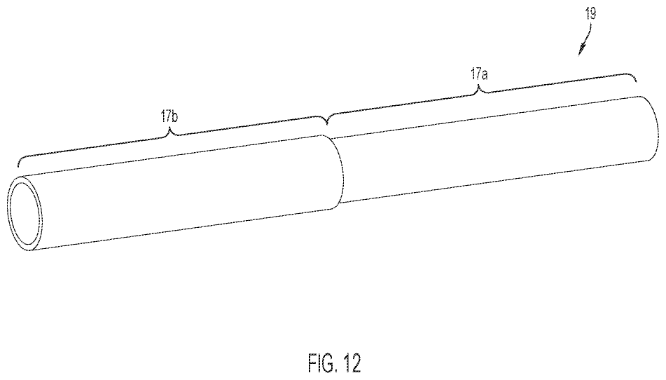

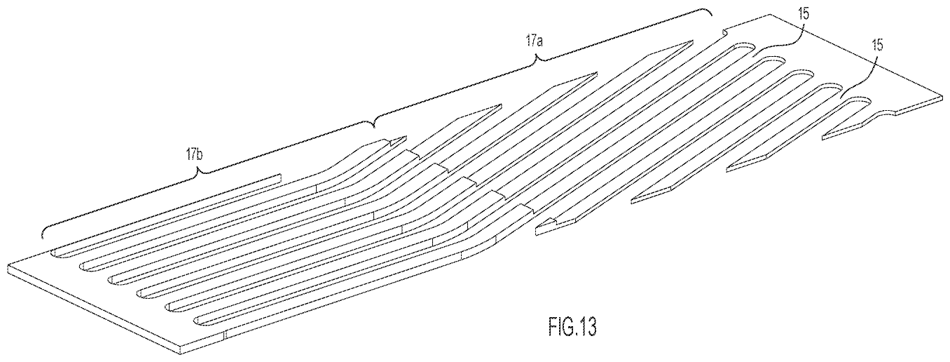

In lieu of, or in combination with providing clot capturing elements having different width dimensions along their length, according to some implementations the thickness of the clot capturing elements 15 may vary along their length as shown in FIGS. 12 and 13. Having clot capturing elements 15 with a varied thickness along their length can have the same or similar effect of providing the clot capturing elements 15 with a varying width as disclosed above. That is, the wider/thicker portions of the clot capturing elements 15 enhance clot capturing capability while the less wide/less thick portions make it easier to cause the clot capturing elements to assume their expanded configuration.

FIG. 12 shows a tube 19 from which a retrieval device is formed by means of a laser cutting operation wherein slots are cut to form a plurality of clot capturing elements 15. In the implementation of FIG. 11, the area in which the proximal end sections 17a of the clot capturing elements are to be produced is thinned. FIG. 13 shows a retrieval device similar to that of FIG. 1C that has been cut along its axial length and laid flat on a surface, the difference between FIG. 1C being that the proximal end sections of clot capturing elements 15 are thinner than the those of the distal end sections 17b.

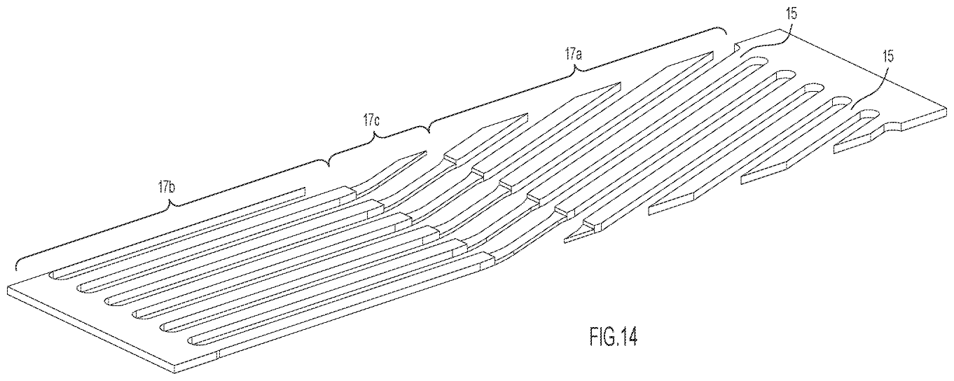

The retrieval device of FIG. 14 is similar to that of FIG. 13, except that only a central region of the clot capturing elements 15 is thinned, resulting in a central section having a thickness less than that of each of proximal and distal end sections 17a and 17b. Because the central section of the clot capturing elements 15 has a reduced thickness (as compared to the thickness of the proximal and distal end sections 17a and 17b), the apex region 6 (see FIG. 10B) of the petal-like structures formed when the retrieval device assumes its expanded state are more flexible than the proximal and distal end sections 17a and 17b. The more flexible central section 17c of the elongate clot capturing elements more gently contact the interior wall of the duct under treatment as the retrieval device is withdrawn therethrough. This can result in better therapeutic outcomes due to less vessel damage occurring during treatment.

To achieve clot capturing elements 15 of reduced thickness, prior to laser cutting a tubular member, a proximal or central end portion of the tubular member may be machined or otherwise acted on to produce a region of reduced thickness as shown in the figures.

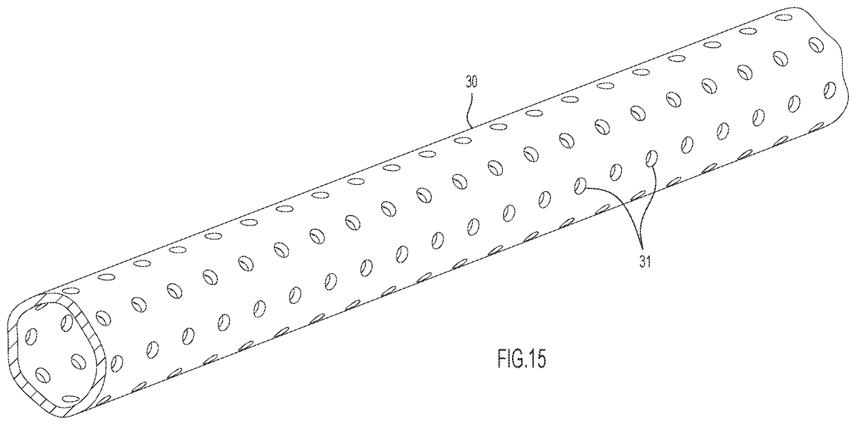

Turning again to FIGS. 1A, 1B, 2A, 2B and 3, the tube 30 is endowed with sufficient flexibility to enable the assembly to be maneuvered through the tortuous anatomy of the patient. According to one implementation, the tube 30 is made of a metal (e.g. stainless steel) with perforations that enhance the longitudinal flexibility of the tube as compared to the tube 30 without perforations. FIG. 15 shows a section of the tube 30 comprising a plurality of perforations 31 that form areas of articulations in the tube to enhance the longitudinal flexibility of the tube.

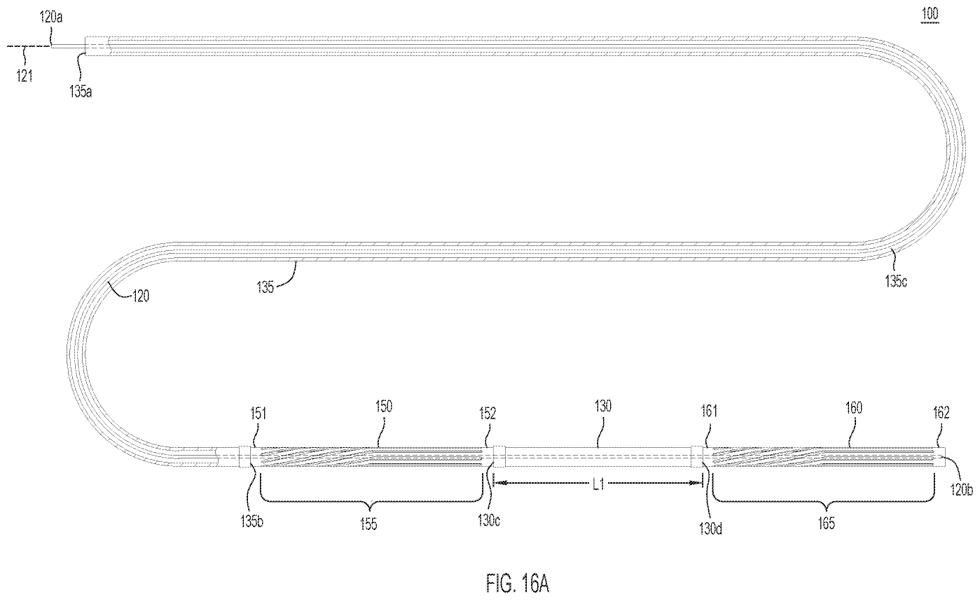

FIG. 16A illustrates an assembly 100 useful in removing obstructions (e.g. blood clots) located within a bodily duct of a patient. The assembly includes first and second retrieval devices 150 and 160 that are each transitional between an unexpanded state as shown in FIG. 16A and a fully expanded state as shown in FIG. 16B. The second retrieval device 160 is disposed distal to the first retrieval device 150. In the implementation shown in FIG. 16A, each of retrieval devices 150 and 160 have the same configuration as the retrieval device 12 shown in FIG. 1C. In the implementation shown in FIG. 16B, each of retrieval devices 150 and 160 has the same configuration as the retrieval device 12 shown in FIGS. 1D and 1E. However, it is important to note that retrieval devices 150 and 160 may comprise other configurations such as the implementations of retrieval devices 12 disclosed above and the retrieval device configurations disclosed in U.S. Pat. No. 10,555,745. Moreover, as will be discussed in more detail below, the configuration of the second retrieval device 160 may differ from the configuration of the first retrieval device 150 in one or both of its nonexpanded and expanded configurations.

The assembly 100 includes an elongate wire 120 having a proximal end 120a, a distal end 120b, and a longitudinal axis 121 on which is mounted the first and second retrieval devices 150 and 160. The first retrieval device 150 includes a proximal collar 151 and a distal collar 152 that each possesses a longitudinal through opening 151b and 152b through which the elongate wire 120 passes. Disposed between the proximal collar 151 and distal collar 152 is a plurality of elongate clot capturing elements 155 that each has a proximal end attached to the proximal collar 151 and a distal end attached to the distal collar 152. The second retrieval device 160 includes a proximal collar 161 and a distal collar 162 that each possesses a longitudinal through opening 161b and 162b through which the elongate wire 120 passes. Disposed between the proximal collar 161 and distal collar 162 is a plurality of circumferentially spaced-apart elongate clot capturing elements 165 that each has a proximal end attached to the proximal collar 161 and a distal end attached to the distal collar 162. The elongate clot capturing elements 155 and 165 may take on a variety of different configurations such as, for example, those disclosed in conjunction with the elongate clot capturing elements 15 previously disclosed.

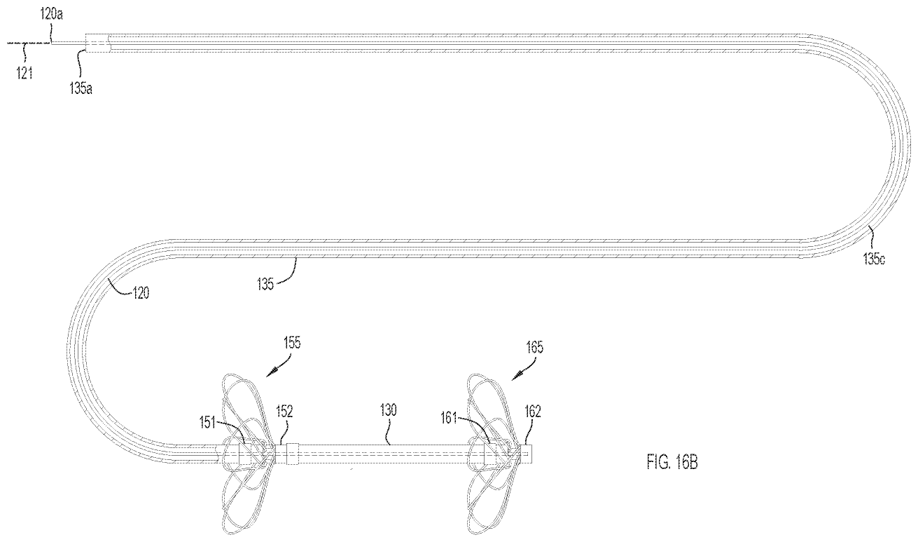

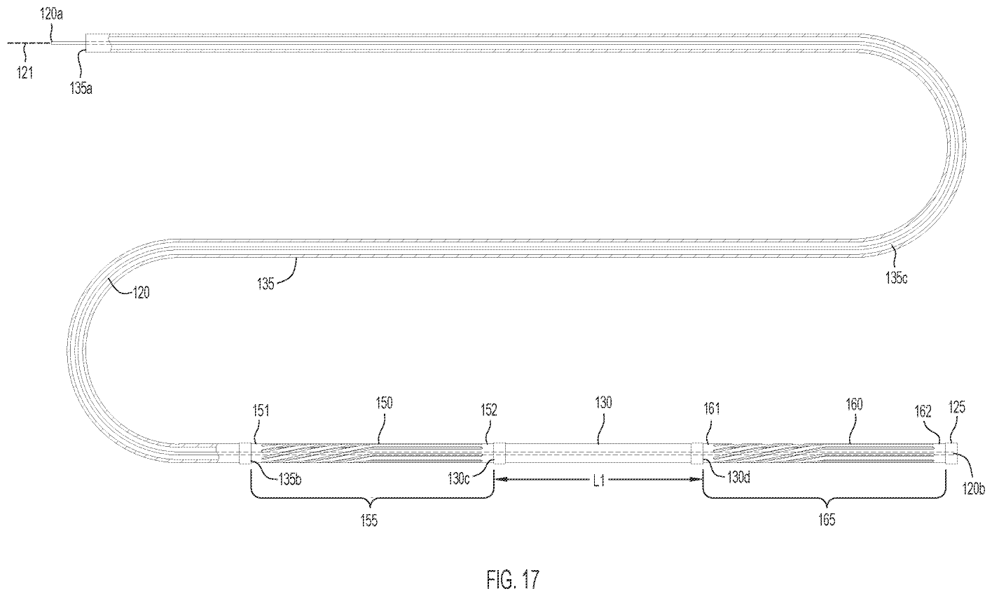

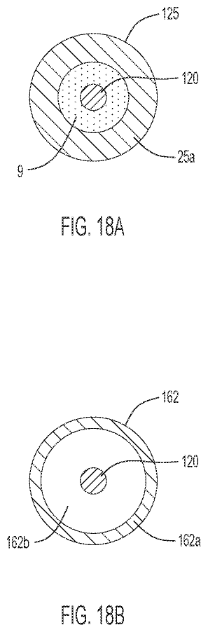

The distal collar 162 of the second retrieval device is operatively coupled with the elongate wire 120. In the implementation of FIGS. 16A and 16B, the distal collar 162 is fixed to the distal end 120b or distal end portion of the elongate wire 120. The distal collar 162 may be fixed to the elongate wire using an adhesive, by soldering, by welding, etc. In an alternative implementation, the distal collar 162 is not fixed to the elongate wire 120. In such implementations, as shown in FIGS. 17A and 17B, a stop 125 is provided on the elongate wire 120. The stop 125 includes a proximal facing surface 125a (FIG. 18A) that faces a distal facing surface 162a (18B) of the distal collar 162. The stop 125 may be formed integrally with the wire 120 or may be secured to the wire with the use of an adhesive 9 as shown in FIG. 18A, or by other fastening methods, such as soldering, welding, etc.

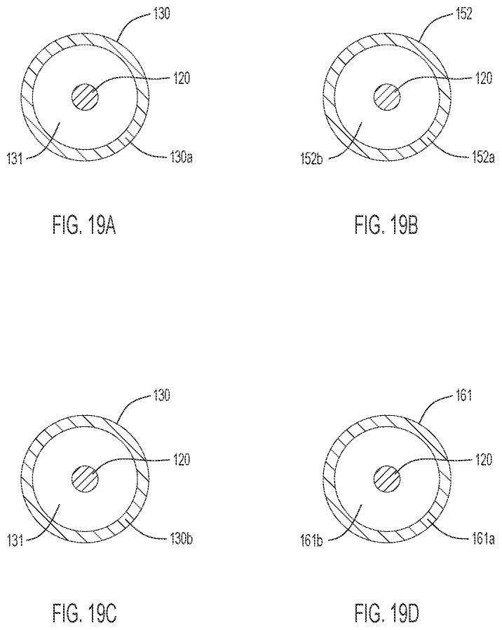

Disposed between the distal collar 152 of the first retrieval device 150 and the proximal collar 161 of the second retrieval device is a first tube 130 having a through passage 131 through which the elongate wire 120 passes. The first tube 130 has a proximal end 130c with a proximal facing surface 130a (FIG. 19A) that faces a distal facing surface 152a (FIG. 19B) of the first retrieval device 150. The first tube 130 also has a distal end 130d with a distal facing surface 130b (FIG. 19C) that faces a proximal facing surface 161a (FIG. 19D) of the second retrieval device 160.

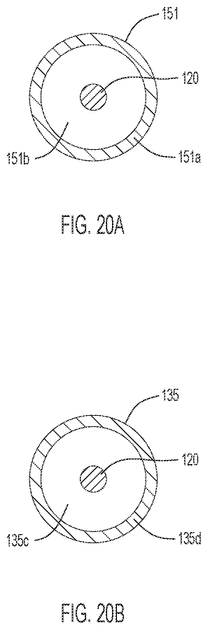

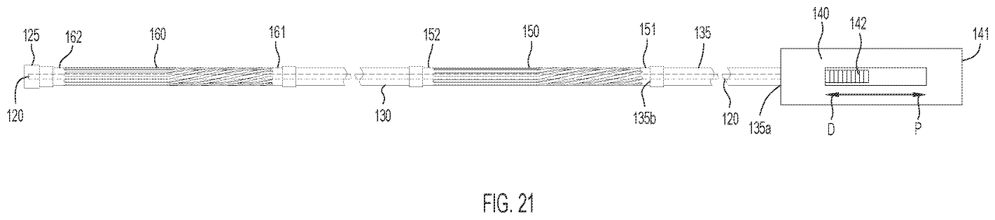

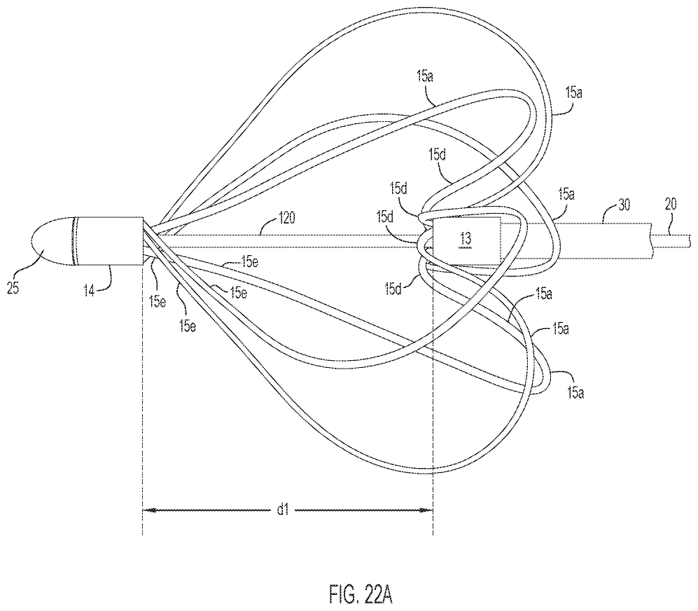

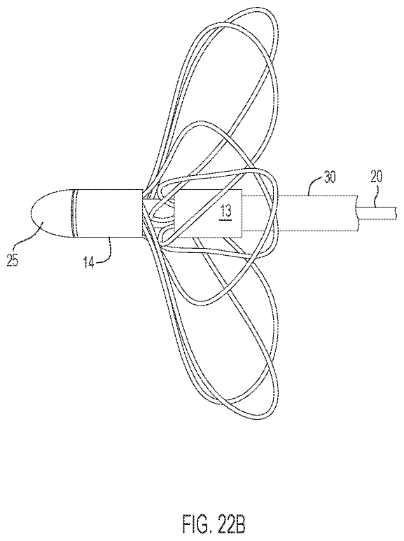

The assembly 100 also includes a second tube 135 mounted on the elongate wire 120. The second tube 135 has a proximal end 135a, a distal end 135b, and a longitudinal through opening/passageway 135c through which the elongate wire 120 passes. According to some implementations, the proximal collar 151 of the first retrieval device 150 is attached to the distal end 135b of the second tube 135 by an adhesive, by soldering, welding, etc. In an alternative implementation, the proximal collar 151 is not fixed to the tube 135 and has a proximal facing surface 151a (FIG. 20A) that faces a distal facing surface 135d (FIG. 20B) of the tube 130. Like discussed above in conjunction with FIGS. 3 and 5C, according to some implementations the distal end 135b of the second tube 135 may be fitted with a ferrule 8 (or other hollow structure) that has a distal facing surface that faces the proximal facing surface 151a of the proximal collar 151 of retrieval device 150.

According to some implementations, the assembly 100 is configured such that each of the first and second retrieval devices 150 and 160 transitions from the unexpanded state to the expanded state at least partially by a withdrawing of the elongate wire 120 proximally (in a direction towards the medical practitioner) while the tube 135 is held stationary. The first and second retrieval devices 150, 160, elongate wire 120 and first and second tubes 130 and 135 are assembled such that as the elongate wire 120 is withdrawn proximally, the longitudinal distance between the proximal collar 151, 161 and distal collar 152, 162 of the first and second retrieval devices 150, 160 decreases to induce a bending and radial extension of the plurality of elongate clot capturing elements 155, 165 as shown in FIG. 16B. As noted above, according to some implementations each of the plurality of elongate clot capturing elements 155 and 165 may be configured to form petal-like structures in which adjacent petal-like structures at least partially overlap with one another. That is, each of the first and second plurality of elongate clot capturing elements 155 and 165 may have the same or similar configuration of the plurality of elongate clot capturing elements 15 shown in FIG. 1E.

According to some implementations, the assembly 100 is also configured such that when the first and second retrieval devices 150, 160 are in the expanded state, they are capable of transitioning from the expanded state towards or to the unexpanded state by a pushing of the elongate wire 120 distally (in a direction away from the medical practitioner). According to such implementations, the first and second retrieval devices 150, 160, elongate wire 120 and first and second tubes 130, 135 are assembled such that as the elongate wire 120 is pushed distally, the distance between the proximal collar 151, 161 and distal collar 152, 162 of the first and second retrieval devices 150, 160 increases.

According to some implementations, the assembly 100 is configured such that each of the first and second retrieval devices 150, 160 transitions from the unexpanded state to the expanded state at least partially by a pushing of the tube 135 distally (in a direction away from the medical practitioner) while the elongate wire 120 is held stationary. According to such implementations, the first and second retrieval devices 150, 160, elongate wire 120 and first and second tubes 130. 135 are assembled such that as the second tube 135 is pushed distally, the proximal collar 151, 161 moves distally so that the axial/longitudinal distance between the proximal collar 151 and distal collar 152 decreases to induce a bending of the plurality of elongate clot capturing elements 155 and so that the axial/longitudinal distance between the proximal collar 161 and distal collar 162 decreases to induce a bending of the plurality of elongate clot capturing elements 165.

According to some implementations, expansion of the first and second retrieval devices 150, 160 occurs with the distal collar 162 of the second retrieval device 160 fixed/attached to the elongate wire 120, the proximal collar 161 of the second retrieval device 160 fixed/attached to the distal end 130d of first tube 130, the distal collar 152 of the first retrieval device 150 fixed/attached to the proximal end 130c of first tube 130, and the proximal collar 151 of the first retrieval device 150 fixed/attached to the distal end 135b of second tube 135. According to some implementations, the first retrieval device 150, the second retrieval device 160 and the first tube 130 comprise a unitary structure, being made from a single piece of material. According to some implementations, the first retrieval device 150, the second retrieval device 160, the first tube 130 and the second tube comprise a unitary structure, being made from a single piece of material.

According to some implementations, expansion of the first and second retrieval devices 150, 160 occurs with the distal collar 162 of the second retrieval device 160 fixed/attached to the elongate wire 120, the proximal collar 161 of the second retrieval device 160 fixed/attached to the distal end 130d of first tube 130, the distal collar 152 of the first retrieval device fixed/attached to the proximal end 130c of first tube 130, and at least a part of the proximal facing surface 151a of proximal collar 151 abutting at least a part of the distal facing surface 135d of the second tube 135 without the proximal collar 151 being fixed/attached to the second tube 135. According to some implementations, the first retrieval device 150, the second retrieval device 160 and the first tube 130 comprise a unitary structure, being made from a single piece of material.

According to some implementations, expansion of the first and second retrieval devices 150, 160 occurs with the distal collar 162 of the second retrieval device 160 not being fixed/attached to the elongate wire 120 and instead with at least a part of the distal facing surface 162a of distal collar 162 abutting at least a part of the proximal facing surface 125a of stop 125, at least a part of the proximal facing surface 161a of proximal collar 161 of the second retrieval device 160 abutting at least a part of the distal facing surface 130 of the first tube without the proximal collar being fixed/attached to the first tube 130, at least a portion of the distal facing surface 152a of distal collar 152 of the first retrieval device 150 abutting at least a part of the proximal facing surface 130a of the first tube 130 without the distal collar 152 being attached to the first tube 130, and at least a part of the proximal facing surface 151a of proximal collar 151 abutting at least a part of the distal facing surface 135d of the second tube 135 without the proximal collar 151 being fixed/attached to the second tube 135.

According to some implementations, expansion of the first and second retrieval devices 150, 160 occurs with the distal collar 162 of the second retrieval device 160 being fixed/attached to the elongate wire 120, at least a part of the proximal facing surface 161a of proximal collar 161 abutting at least a part of the distal facing surface 130b of the first tube 130 without the proximal collar 161 being fixed/attached to the first tube 130, at least a part of the distal facing surface 152a of distal collar 152 abutting at least a part of the proximal facing surface 130a of the first tube 130 without the distal collar 152 collar being fixed/attached to the first tube 130, and the proximal collar 151 of the first retrieval device 150 fixed/attached to the distal end 135b of second tube 135.

According to some implementations, expansion of the first and second retrieval devices 150, 160 occurs with the distal collar 162 of the second retrieval device 160 not being fixed/attached to the elongate wire 120 and instead with at least a part of the distal facing surface 162a of distal collar 162 abutting at least a part of the proximal facing surface 125a of stop 125, at least a part of the proximal facing surface 161a of proximal collar 161 of the second retrieval device 160 abutting at least a part of the distal facing surface 130 of the first tube without the proximal collar being fixed/attached to the first tube 130, at least a portion of the distal facing surface 152a of distal collar 152 of the first retrieval device 150 abutting at least a part of the proximal facing surface 130a of the first tube 130 without the distal collar 152 being attached to the first tube 130, and the proximal collar 151 of the first retrieval device 150 fixed/attached to the distal end 135b of second tube 135.

According to some implementations, expansion of the first and second retrieval devices 150, 160 occurs with the distal collar 162 of the second retrieval device 160 fixed/attached to the elongate wire 120, at least a part of the proximal facing surface 161a of proximal collar 161 of the second retrieval device 160 abutting at least a part of the distal facing surface 130 of the first tube without the proximal collar being fixed/attached to the first tube 130, at least a portion of the distal facing surface 152a of distal collar 152 of the first retrieval device 150 abutting at least a part of the proximal facing surface 130a of the first tube 130 without the distal collar 152 being attached to the first tube 130, and at least a part of the proximal facing surface 151a of proximal collar 151 abutting at least a part of the distal facing surface 135d of the second tube 135 without the proximal collar 151 being fixed/attached to the second tube 135.

According to some implementations, expansion of the first and second retrieval devices 150, 160 occurs with the distal collar 162 of the second retrieval device 160 not being fixed/attached to the elongate wire 120 and instead with at least a part of the distal facing surface 162a of distal collar 162 abutting at least a part of the proximal facing surface 125a of stop 125, the proximal collar 161 of the second retrieval device 160 fixed/attached to the distal end 130d of first tube 130, the distal collar 152 of the first retrieval device fixed/attached to the proximal end 130c of first tube 130, and at least a part of the proximal facing surface 151a of proximal collar 151 abutting at least a part of the distal facing surface 135d of the second tube 135 without the proximal collar 151 being fixed/attached to the second tube 135. According to some implementations, the first retrieval device 150, the second retrieval device 160 and the first tube 130 comprise a unitary structure, being made from a single piece of material.

According to some implementations, expansion of the first and second retrieval devices 150, 160 occurs with the distal collar 162 of the second retrieval device 160 not being fixed/attached to the elongate wire 120 and instead with at least a part of the distal facing surface 162a of distal collar 162 abutting at least a part of the proximal facing surface 125a of stop 125, the proximal collar 161 of the second retrieval device 160 fixed/attached to the distal end 130d of first tube 130, the distal collar 152 of the first retrieval device fixed/attached to the proximal end 130c of first tube 130, and the proximal collar 151 of the first retrieval device 150 fixed/attached to the distal end 135b of second tube 135. According to some implementations, the first retrieval device 150, the second retrieval device 160 and the first tube 130 comprise a unitary structure, being made from a single piece of material.

According to some implementations, expansion of the first and second retrieval devices 150, 160 occurs with the distal collar 162 of the second retrieval device 160 being fixed/attached to the elongate wire 120, the proximal collar 161 of the second retrieval device 160 fixed/attached to the distal end 130d of first tube 130, at least a part of the distal facing surface 152a of distal collar 152 abutting at least a part of the proximal facing surface 130a of the first tube 130 without the distal collar 152 collar being fixed/attached to the first tube 130, and the proximal collar 151 of the first retrieval device 150 fixed/attached to the distal end 135b of second tube 135. According to some implementations, the second retrieval device 160 and the first tube 130 comprise a unitary structure, being made from a single piece of material.

According to some implementations, expansion of the first and second retrieval devices 150, 160 occurs with the distal collar 162 of the second retrieval device 160 being fixed/attached to the elongate wire 120, the proximal collar 161 of the second retrieval device 160 fixed/attached to the distal end 130d of first tube 130, at least a part of the distal facing surface 152a of distal collar 152 abutting at least a part of the proximal facing surface 130a of the first tube 130 without the distal collar 152 collar being fixed/attached to the first tube 130, and at least a part of the proximal facing surface 151a of proximal collar 151 abutting at least a part of the distal facing surface 135d of the second tube 135 without the proximal collar 151 being fixed/attached to the second tube 135. According to some implementations, the second retrieval device 160 and the first tube 130 comprise a unitary structure, being made from a single piece of material.

According to some implementations, expansion of the first and second retrieval devices 150, 160 occurs with the distal collar 162 of the second retrieval device 160 fixed/attached to the elongate wire 120, at least a part of the proximal facing surface 161a of proximal collar 161 of the second retrieval device 160 abutting at least a part of the distal facing surface 130 of the first tube without the proximal collar being fixed/attached to the first tube 130, the distal collar 152 of the first retrieval device 150 fixed/attached to the proximal end 130c of first tube 130, and at least a part of the proximal facing surface 151a of proximal collar 151 abutting at least a part of the distal facing surface 135d of the second tube 135 without the proximal collar 151 being fixed/attached to the second tube 135.

According to some implementations, expansion of the first and second retrieval devices 150, 160 occurs with the distal collar 162 of the second retrieval device 160 fixed/attached to the elongate wire 120, at least a part of the proximal facing surface 161a of proximal collar 161 of the second retrieval device 160 abutting at least a part of the distal facing surface 130 of the first tube without the proximal collar being fixed/attached to the first tube 130, the distal collar 152 of the first retrieval device 150 fixed/attached to the proximal end 130c of first tube 130, and the proximal collar 151 of the first retrieval device 150 fixed/attached to the distal end 135b of the second tube 135. According to some implementations, the first retrieval device 150 and the second tube 135 comprise a unitary structure, being made from a single piece of material.

According to some implementations, expansion of the first and second retrieval devices 150, 160 occurs with the distal collar 162 of the second retrieval device 160 not being fixed/attached to the elongate wire 120 and instead with at least a part of the distal facing surface 162a of distal collar 162 abutting at least a part of the proximal facing surface 125a of stop 125, the proximal collar 161 of the second retrieval device 160 fixed/attached to the distal end 130d of first tube 130, at least a part of the distal facing surface 152a of distal collar 152 abutting at least a part of the proximal facing surface 130a of the first tube 130 without the distal collar 152 collar being fixed/attached to the first tube 130, and the proximal collar 151 of the first retrieval device 150 fixed/attached to the distal end 135b of second tube 135.

According to some implementations, expansion of the first and second retrieval devices 150, 160 occurs with the distal collar 162 of the second retrieval device 160 not being fixed/attached to the elongate wire 120 and instead with at least a part of the distal facing surface 162a of distal collar 162 abutting at least a part of the proximal facing surface 125a of stop 125, the proximal collar 161 of the second retrieval device 160 fixed/attached to the first tube 130, at least a portion of the distal facing surface 152a of distal collar 152 of the first retrieval device 150 abutting at least a part of the proximal facing surface 130a of the first tube 130 without the distal collar 152 being attached to the first tube 130, and at least a part of the proximal facing surface 151a of proximal collar 151 abutting at least a part of the distal facing surface 135d of the second tube 135 without the proximal collar 151 being fixed/attached to the second tube 135.

According to some implementations, expansion of the first and second retrieval devices 150, 160 occurs with the distal collar 162 of the second retrieval device 160 not being fixed/attached to the elongate wire 120 and instead with at least a part of the distal facing surface 162a of distal collar 162 abutting at least a part of the proximal facing surface 125a of stop 125, at least a part of the proximal facing surface 161a of proximal collar 161 of the second retrieval device 160 abutting at least a part of the distal facing surface 130 of the first tube without the proximal collar being fixed/attached to the first tube 130, the distal collar 152 of the first retrieval device 150 being attached to the first tube 130, and at least a part of the proximal facing surface 151a of proximal collar 151 abutting at least a part of the distal facing surface 135d of the second tube 135 without the proximal collar 151 being fixed/attached to the second tube 135.

According to some implementations, expansion of the first and second retrieval devices 150, 160 occurs with the distal collar 162 of the second retrieval device 160 not being fixed/attached to the elongate wire 120 and instead with at least a part of the distal facing surface 162a of distal collar 162 abutting at least a part of the proximal facing surface 125a of stop 125, at least a part of the proximal facing surface 161a of proximal collar 161 of the second retrieval device 160 abutting at least a part of the distal facing surface 130 of the first tube without the proximal collar being fixed/attached to the first tube 130, the distal collar 152 of the first retrieval device 150 being attached to the first tube 130, and the proximal collar 151 of the first retrieval device 150 fixed/attached to the distal end 135b of second tube 135. According to some implementations, the first retrieval device 150 and the second tube 135 comprise a unitary structure, being made from a single piece of material.

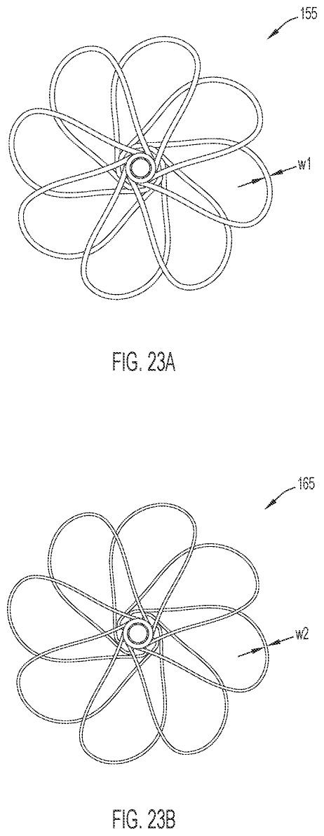

Inducing axial movement of the elongate wire 120 and/or the tube 135 to cause the first and second retrieval devices 150 and 160 to transition between the nonexpanded and expanded states may come in the form of a hand-held device that may be operated by the medical practitioner with the use of a single hand. FIG. 21 depicts an example actuation device 140 that includes an external housing 141 that is configured to be gripped by the hand of the medical practitioner. According to one implementation, the actuation device 140 includes a sliding member 142 to which is attached or coupled to the proximal end 120a or to a proximal end portion of the elongate wire 120. According to such an implementation, a proximal end 135a of tube 135 may be coupled or attached to a fixed part of the actuation device 140, such as a part of the housing 141. According to some implementations, the actuation device 140 is configured such that the sliding member 142 is capable of moving proximally in the P direction and distally in the D direction as shown in FIG. 21. According to one implementation, the starting position of the sliding member 142 is the forward-most position as shown in FIG. 21 and this position corresponds to the first and second retrieval devices 150 and 160 being in the unexpanded state. In use, upon the first and second retrieval device having been delivered to a location distal to an obstruction in the duct of the patient, the medical practitioner may act on the sliding member 142 with his/her thumb to move the sliding member proximally. This causes a withdrawing of the elongate wire 120 proximally, which by virtue of the distal collar 162 of the second retrieval device 160 being operatively coupled with the elongate wire 120, results in the distal collar 162 advancing proximally nearer the proximal collar 161 and distal collar 152 advancing proximally nearer the proximal collar 151. As explained above, this results in a bending of the elongate clot capturing elements 155 and 165 so that they assume a looped or petal-like configuration sufficient to ensnare an obstruction. According to some implementations, the actuation device 140 is configured such that when the sliding member 142 is in the proximal-most position, each of the first and second retrieval devices 150 and 160 is in a fully expanded state.

According to some implementations, it may be beneficial to cause the first and second retrieval devices 150 and 160 to assume a partial expanded state or a fully unexpanded state after an obstruction has been captured. In such cases, the sliding member 142 may be advanced distally to cause the first and second retrieval devices to assume the partial expanded state or fully unexpanded state.

As noted above, according to some implementations the first and second retrieval devices 150 and 160 may be expanded by holding the elongate wire 120 stationary while advancing the second tube 135 distally. This results in the distal facing surface 135d of the second tube 135 pushing against the proximal facing surface 151a of the proximal collar 151 of the first retrieval device 150 to cause the proximal collar 151 to advance distally towards the distal collar 152 so that the first retrieval device assumes the expanded state. By virtue of their relationship with the proximal collar 151, the first tube 130 and the proximal collar 161 of the second retrieval device 160 also advance distally so that the second retrieval device assuming the expanded state. According to such implementations, the proximal end 120a or a proximal end portion of the elongate wire 120 is fixed to a stationary part of the actuation device 140 and the proximal end of the second tube 135 is attached to or otherwise coupled to the sliding member 142.

According to other implementations, the actuation device 140 may be configured to cause an axial movement of both the elongate wire 120 and the second tube 135 in opposite directions to cause the first and second retrieval devices 150 and 160 to transition to their expanded states.

According to other implementations, a two-handed approach may be used to cause the first and second retrieval devices 150 and 160 to transition between their unexpanded and expanded states. This may be accomplished, for example, by holding one of the elongate wire 120 and second tube 135 stationary while applying axial movement to the other.

According to some implementations, the first and second retrieval devices 150, 160 may be constructed to transition from the unexpanded state to the expanded state only by applying axially directed forces to one or both of their respective proximal and distal collars 151, 161 and 152, 162 as discussed above. One or both of the first and second retrieval devices 150, 160 be made of a shape memory alloy (e.g. Nitinol) and configured to self-expand to at least a partially expanded state. According such implementations, a sheath may be used to constrain the clot capturing elements 155 and/or 165 prior to deploying the first and second retrieval devices inside the duct of the patient. At the time of deployment, the sheath is removed and an axial force is applied to the proximal and distal collars of the first and second retrieval devices to cause them to assume a partially or fully expanded state. One or both of the first and second retrieval devices 150, 160 may assume a partially expanded state like that shown in FIG. 7. A benefit of having a retrieval device at least partially self-expand, is that it reduces the initial amount of axial force needed to be applied to one or both of the distal and proximal collars during an initial expansion of the clot capturing elements. As explained above, this makes it easier for the medical practitioner to deploy the retrieval device and also reduces the risk of breakage. For example, because the initial amount of axial force needed to be applied is reduced, it is less likely the elongate wire 120 will decouple from the distal collar 162 of the second retrieval device 160 or decouple from stop 125 as it is withdrawn proximally for the purpose of causing one or both of the first and second retrieval devices 150, 160 to assume its expanded state. Shaping of the elongate clot capturing elements 155 and/or 165 to cause them to at least partially self-expand may include the method disclosed above in conjunction with the description of FIG. 7.

Like the elongate clot capturing elements 15 of retrieval device 12 disclosed above, according to some implementations the elongate clot capturing elements 155 and 165 of retrieval devices 150 and 160 may be formed by cutting slots in a tubular member like that shown in FIG. 1C. Methods other than laser cutting a tube may also be employed to construct the retrieval devices 150 and 160. Other such methods are disclosed in U.S. Pat. No. 10,555,745, issued Feb. 11, 2020, which is incorporated by reference herein in its entirety. Furthermore, as disclosed above, the retrieval devices disclosed and contemplated herein may further be constructed to comprise elongate clot capturing elements shaped and oriented like those disclosed in U.S. Pat. No. 10,555,745. These include, in an unlimited way, the examples of FIGS. 8A-B, 8E, 9B, 10A-B, 12, 13A-B, 14A-B, 15A-C, 16A-B, 17A-B and 18A-B of U.S. Pat. No. 10,555,745.

In U.S. Pat. No. 10,555,745 a number of implementations are disclosed in which the elongate clot capturing elements of one or more retrieval devices self-expand from a radially constrained unexpanded state to a radially unconstrained expanded state. According to some implementations the one or more retrieval devices are held in the radially constrained position by a sheath that is removed to enable its or their expansion. According to some implementations, the one or more retrieval devices are radially constrained in the unexpanded state by the internal wall of a delivery catheter which is withdrawn to expose the one or more retrieval devices after having been delivered to a location distal to the treatment site. The written descriptions of FIGS. 1A-3 and 19A-D of U.S. Pat. No. 10,555,745 disclose such implementations.

As also disclosed in U.S. Pat. No. 10,555,745, according to some implementations when the one or more retrieval devices are in a radially unconstrained state, a space exists between their respective proximal and distal collars. Regarding the present disclosure, retrieval devices 12, 150 and 160 may comprise the self-expanding characteristics of at least some of the retrieval devices disclosed in U.S. Pat. No. 10,555,745, whereby the manual actuation assemblies 40 and 140 disclosed above may be used to urge the retrieval devices into a more fully expanded state by acting on the proximal and/or distal collars of the retrieval devices to reduce the longitudinal distance between the proximal and distal collars after the retrieval devices have been deployed inside the duct of the patient.

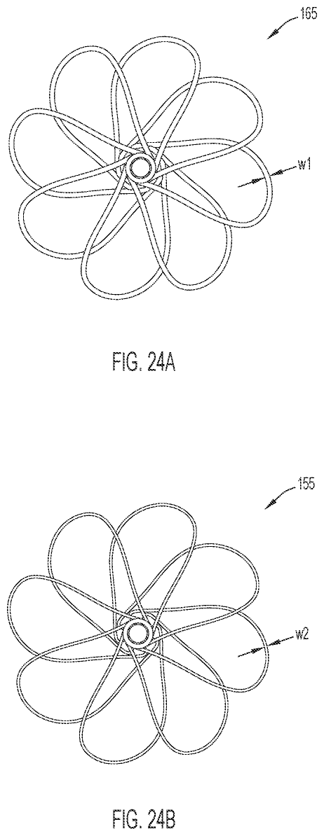

FIG. 22A shows the retrieval device 12 according to one implementation in which it self-expands from a radially constrained state to a radially unconstrained state. The radially unconstrained state as shown in FIG. 22A can be with the retrieval device 12 resting on a surface (e.g. a benchtop) or suspended in air. As shown, in the radially unconstrained state there is a distance d1 between the proximal collar 13 and distal collar 14. According to some implementations, the elongate wire 20 and/or tube 30 may be moved axially in any manner disclosed above to effectuate a decrease in the distance d1 between the proximal and distal collars 13, 14 such that the retrieval device 12 assumes a configuration like or similar to that shown in FIG. 1D. In FIG. 1D and also FIG. 22B, retrieval device 12 is in a fully expanded state (a state in which a distance between the proximal collar 13 and distal collar 14 is physically at a minimum). According to some implementations, as shown in FIG. 22B, the minimum distance is achieved upon at least some of the proximal end sections 15d of the clot capturing elements 15a abutting at least some of the distal end sections 15e of the clot capturing elements 15a.

According to some implementations, the elongate clot capturing elements 155 of the first retrieval device 150 have a width and/or a thickness that is different that the respective width and/or thickness of the elongate clot capturing elements 165 of the second retrieval device 160. According to some implementations, the width and/or thickness dimensions of the elongate clot capturing elements are selected such that the elongate clot capturing elements of one of the first and second retrieval devices is stiffer than the elongate clot capturing elements of the other. According to one such implementation, the elongate clot capturing elements 155 of the first retrieval device 150 having a first bending stiffness that is greater than a second bending stiffness of the elongate clot capturing elements 165 of the second retrieval device 160. According to another implementation, the elongate clot capturing elements 165 of the second retrieval device 160 has a first bending stiffness that is greater than a second bending stiffness of the elongate clot capturing elements 155 of the first retrieval device 150. In each of these cases, the elongate clot capturing elements having the first bending stiffness are more resistant to bending than the elongate clot capturing elements having the second bending stiffness. In the case of the elongate clot capturing elements 155 of the first retrieval device 150 having the first bending stiffness, the first retrieval device can more aggressively entrap the obstruction to be removed from the duct of the patient, and the clot capturing elements 165 of the second retrieval device 160 can follow behind the first retrieval device 150 to more gently capture a remaining portion of the obstruction not captured by the first retrieval device 150. In the case of the elongate clot capturing elements 165 of the second retrieval device 160 having the first bending stiffness, the second retrieval device can more aggressively entrap the obstruction to be removed from the duct of the patient, and the clot capturing elements 155 of the first retrieval device 150 can precede the second retrieval device 150 to more gently engage with the obstruction. In each of these cases the first and second retrieval devices 150, 160 can be tailored to optimize the capture of different types of obstructions. In each of these cases damage to the vessel/duct wall of the patient may also be minimized.

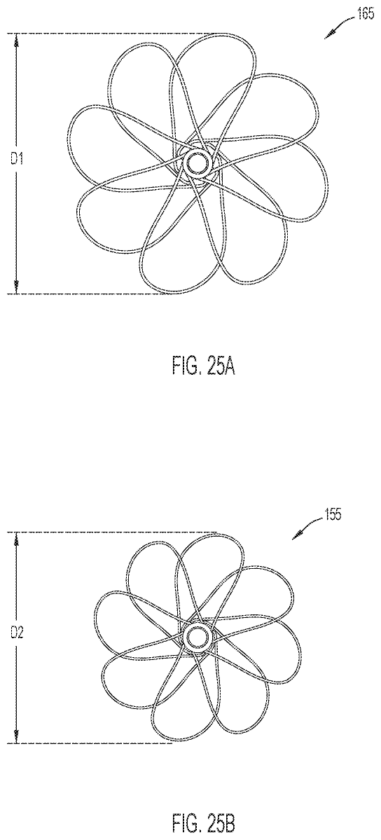

FIG. 23A shows a front view of the first retrieval device 150 with the elongate clot capturing elements 155 having a first width w1. FIG. 23B shows a front view of the second retrieval device 160 with the elongate clot capturing elements 165 having a second width w2, less than the first width w1.

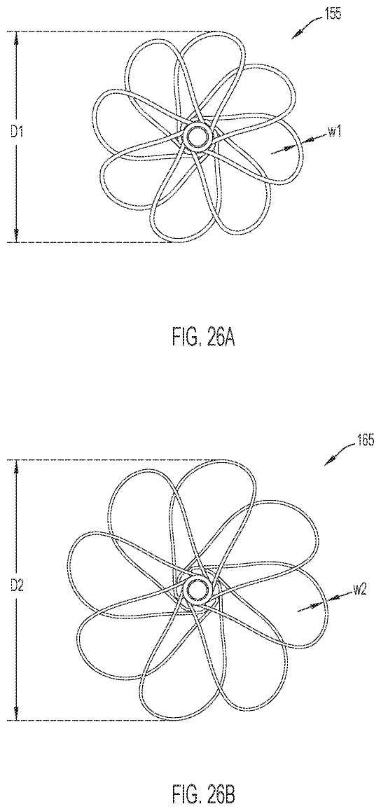

FIG. 24A shows a front view of the second retrieval device 160 with the elongate clot capturing elements 165 having a first width w1. FIG. 24B shows a front view of the first retrieval device 150 with the elongate clot capturing elements 155 having a second width w2, less than the first width w1.

Other ways in which the elongate clot capturing elements 155, 165 of the first and second retrieval devices 150, 160 may be endowed with different bending stiffness characteristics is by: 1) providing the elongate clot capturing elements 155 of the first retrieval device 150 with a thickness that is greater than or less than the elongate clot capturing elements 165 of the second retrieval device 160; 2) making the elongate clot capturing elements 155 of the first retrieval device 150 of a first material that is more or less rigid than a second material from which the elongate clot capturing elements 165 of the second clot capturing device 160 are made; 3) making the elongate clot capturing elements of each of the first and second retrieval devices 150 and 160 from a same material and heat treating one set (155 or 165) of the elongate clot capturing elements to harden the material; 4) coating one set (155 or 165) of the elongate clot capturing elements with a material (e.g. metal material, polymeric material, etc.) and 5) using any combination of the methods disclosed above.

According to some implementations, when the first and second retrieval devices are in their respective fully expanded state, one of the first and second retrieval devices has a fully expanded diameter greater than the fully expanded diameter of the other. As shown in FIGS. 25A and 25B, according to one such implementation the second retrieval device 160 (FIG. 25A) has a fully expanded diameter D1 greater than the fully expanded diameter D2 of the first retrieval device 150 (FIG. 25B). In the latter case, the smaller diameter first retrieval device 150 can be used to capture only a portion of the clot/obstruction, making it easier for the second retrieve 160 to follow and capture a remaining portion of the clot/obstruction.

According to some implementations, the elongate clot capturing elements of the first and second retrieval devices differ in bending stiffness in addition to the first and second retrieval devices having different expanded diameters. For example, according to one implementation the elongate clot capturing elements 155 of the first retrieval device 150 are stiffer than those of the second retrieval device 160 and the second retrieval device 160 has a fully expanded diameter D2 greater than the fully expanded diameter D1 of the first retrieval device 150. FIGS. 26A and 26B illustrate this latter case wherein the elongate clot capturing elements 155 of the first retrieval device 150 (FIG. 26A) are endowed with a greater bending stiffness than the elongate clot capturing elements 165 of the second retrieval device 160 by virtue of its elongate clot capturing elements 155 having a greater width w1 than the width w2 of the elongate clot capturing elements of the second retrieval device 160 (FIG. 26B).

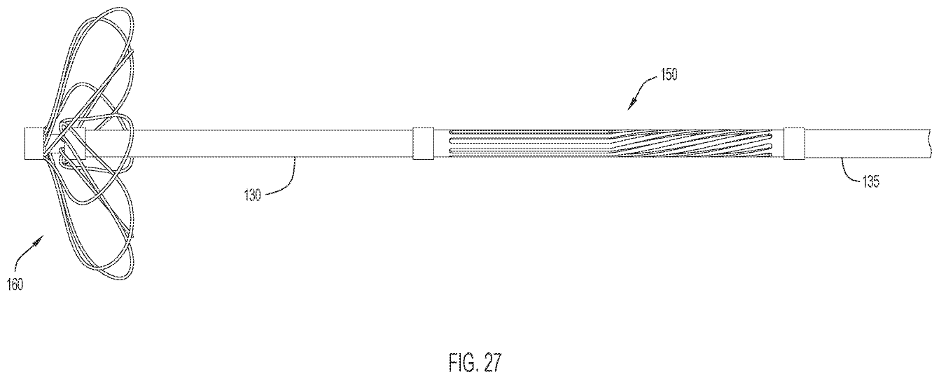

According to some implementations the stiffness characteristics of the elongate clot capturing elements 155 and 165 differ in a way that results in the second retrieval device 160 expanding before the first retrieval device 150, or vice versa. For example, in the implementation of FIGS. 23A and 23B, the widths w1 and w2 of the elongate clot capturing elements 155,165 may vary in a way that results in the second retrieval device 160 expanding before the first retrieval device 150 expands as shown in FIG. 27. FIG. 27 shows the second retrieval device 160 being in its fully expanded state before the first retrieval device 150 begins to expand. According to other implementations, the widths w1 and w2 of the elongate clot capturing elements 155 and 165 may vary in a way that results in the second retrieval device 160 partially expanding before the first retrieval device 150 begins to expand.

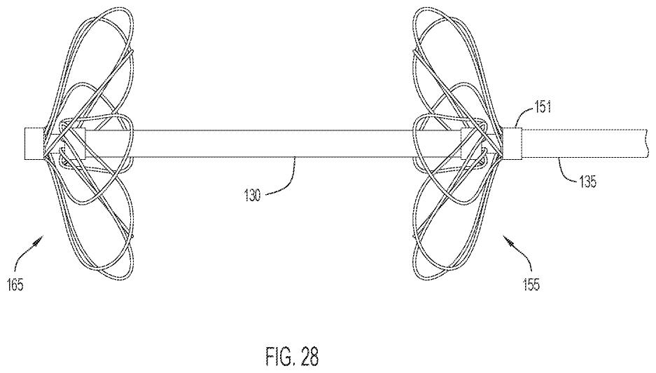

In the implementations of FIGS. 16A-26B the distal collar 152 of the first retrieval device 150 and the proximal collar 161 of the second retrieval device 160 are separated by a tube 130 that has a length L1 sufficient to prevent the plurality of elongate clot capturing elements 155 of the second retrieval device 160 from touching the elongate clot capturing elements 165 when each of the first and second retrieval devices is in its fully expanded state. However, as will be discussed in detail below, according to some implementations a tandem arrangement of first and second retrieval devices in which at least some (or all) of the elongate clot capturing elements of the second retrieval device abut against at least some (or all) of the elongate clot capturing elements of the first retrieval device for the purpose of inhibiting the elongate clot capturing elements of the first retrieval device from inverting backwards (like an umbrella in a strong wind) and losing an entrapped clot/obstruction. FIG. 28 illustrates the first retrieval device 155 with there existing a backward inversion of the clot capturing elements 155. The backward inversion can occur when clot capturing elements are advanced proximally through an obstruction of high rigidity. To address this issue, any of a number of techniques disclosed above may be employed to stiffen the elongate clot capturing elements.

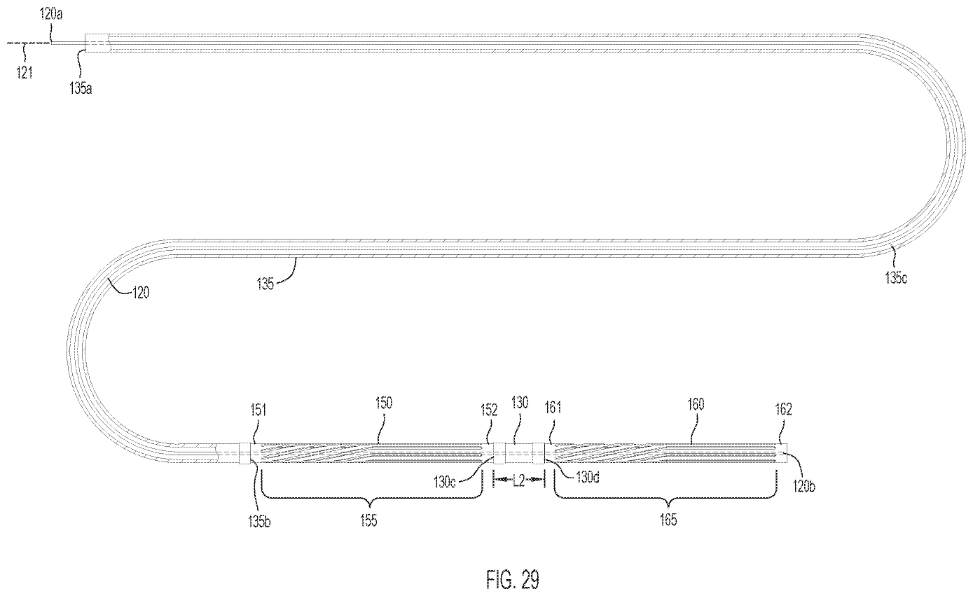

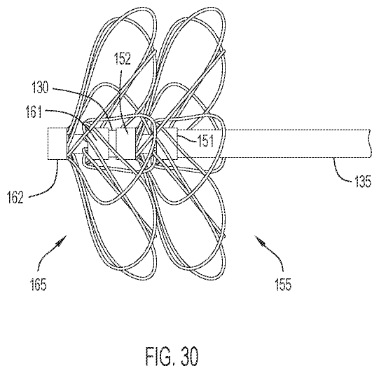

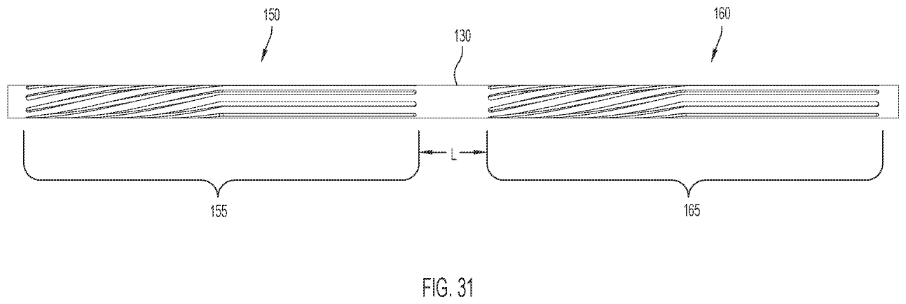

Another method to prevent a backward inversion is to buttress the elongate clot capturing elements of a first retrieval with the elongate clot capturing elements of a second retrieval device that is disposed distal to the first retrieval device. FIG. 29 is a variant of the assembly of FIG. 16A with the length of the first tube 130 being shorter. The manner in which the first and second retrieval devices 150 and 160 transition between their non-expanded and expanded states is the same or similar to that disclosed above. However, the length L2 of the first tube 130 is sufficiently short to enable at least some of the clot capturing elements 165 of the second retrieval device to abut at least some of the clot capturing elements 155 of the first retrieval device 150 when the first and second retrieval devices are in their fully expanded states as shown in FIG. 30. According to some implementations, the first retrieval device 150, second retrieval device 160 and first tube 130 are made from a single piece of material as shown in FIG. 31.

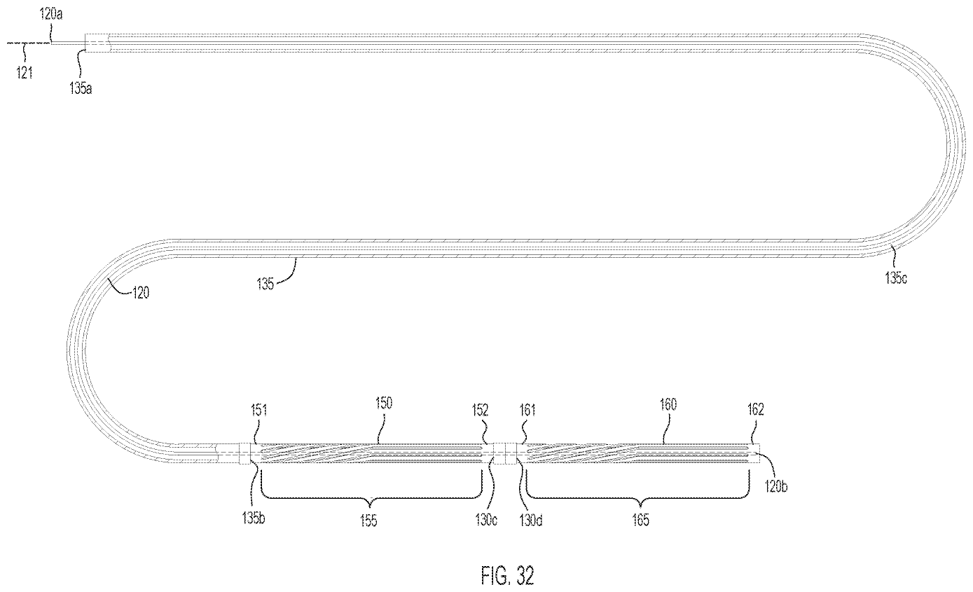

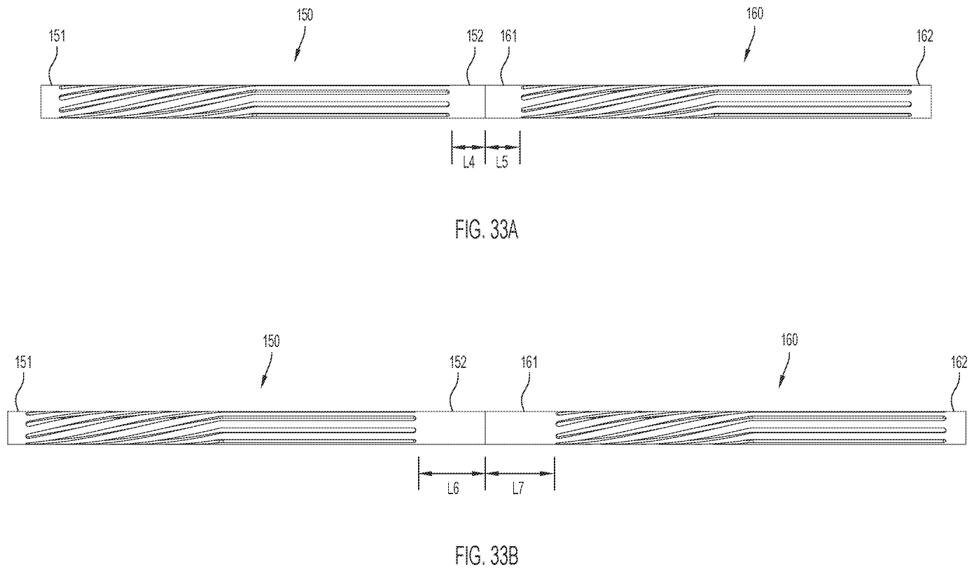

FIG. 32 shows a similar configuration to that shown in FIG. 29 wherein the proximal end of retrieval device 160 is attached to or abuts (without attachment) the distal end of the first retrieval device 150 without a separate tube being disposed between them. In implementations where the first and second retrieval devices are attached to one another, the first and second retrieval devices may be a unitary structure, being made from a single piece of material.

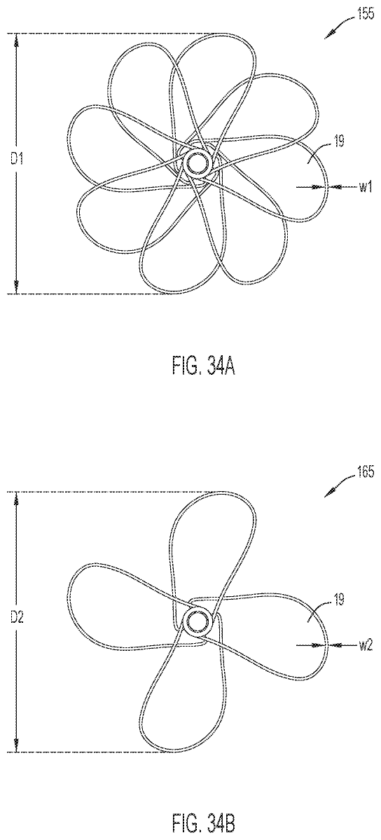

With continued reference to FIG. 32, FIGS. 33A and 33B show how the length L4 and L6 of the distal collar 152 of the first retrieval device 150 and the length L5 and L7 of the proximal collar 161 of the second retrieval device 160 may be varied to achieve a desired interaction between elongate clot capturing element 155 and 165 for the purpose of inhibiting a backward inversion of the clot capturing elements 155 of the first retrieval device 150. According to other implementations the length of distal collar 152 and proximal collar 161 are selected such that there is no interaction between the elongate clot capturing elements 155, 165 of the first and second retrieval devices 150, 160 when they are in their fully expanded states.

In the example retrieval devices disclosed above, when the retrieval devices are in their fully expanded state their elongate clot capturing elements are configured to form eight petal-like/looped structures 19 like those shown for example in FIG. 1E. According to other implementations the retrieval devices may have more or fewer than eight petal-like/loop structures. Moreover, according to some implementations when a clot retrieval assembly includes multiple clot retrieval devices, like the assembly of FIGS. 16A and 16B, the clot retrieval devices may have a different number of elongate clot capturing elements such that they possess a different number of petal-like/looped structures when in their fully expanded states. In this way, an aggregate clot capturing capability of the multiple retrieval devices can be tuned to achieve a desired result optimized to the location and type of obstruction to be retrieved.

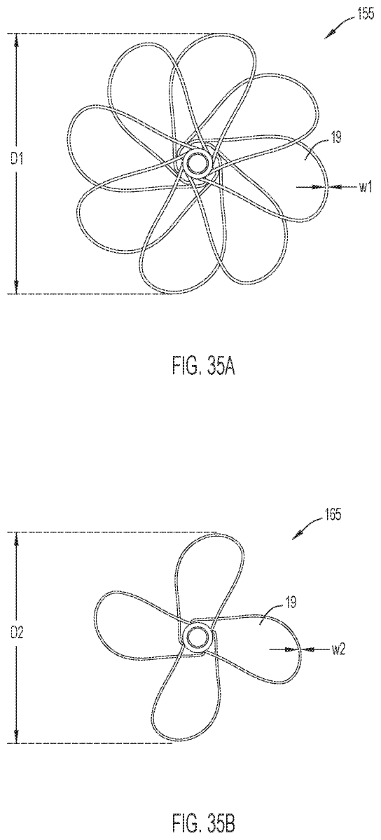

FIGS. 34A and 34B show an example in which the elongate clot capturing elements 155 and 165 of the first and second retrieval devices 150,160 have a different number of petal-like/looped structures 19.

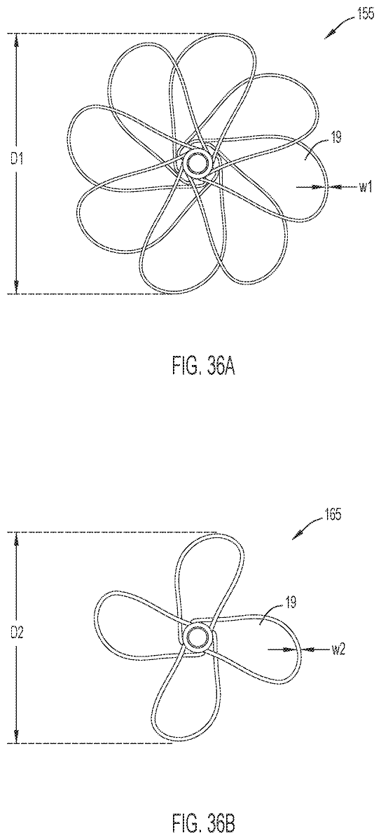

FIGS. 35A and 35B show an example in which the elongate clot capturing elements 155 and 165 of the first and second retrieval devices 150,160 have a different number of petal-like/looped structures and different fully expanded diameters (D1 being greater than D2).

FIGS. 36A and 36B show an example in which the elongate clot capturing elements 155 and 165 of the first and second retrieval devices 150,160 have a different number of petal-like/looped structures, different fully expanded diameters (D1 being greater than D2) and different widths (w2 being greater than w1)

Any of the features of FIGS. 34A-B, 35A-B and 36A-B are applicable to the implementations of FIGS. 29-33B disclosed above. That is, the first and second retrieval devices 150 and 160 of FIGS. 29-33B may possess the features of the first and second retrieval devices 150 and 160 depicted in FIGS. 34A-B, 35A-B and 36A-B.

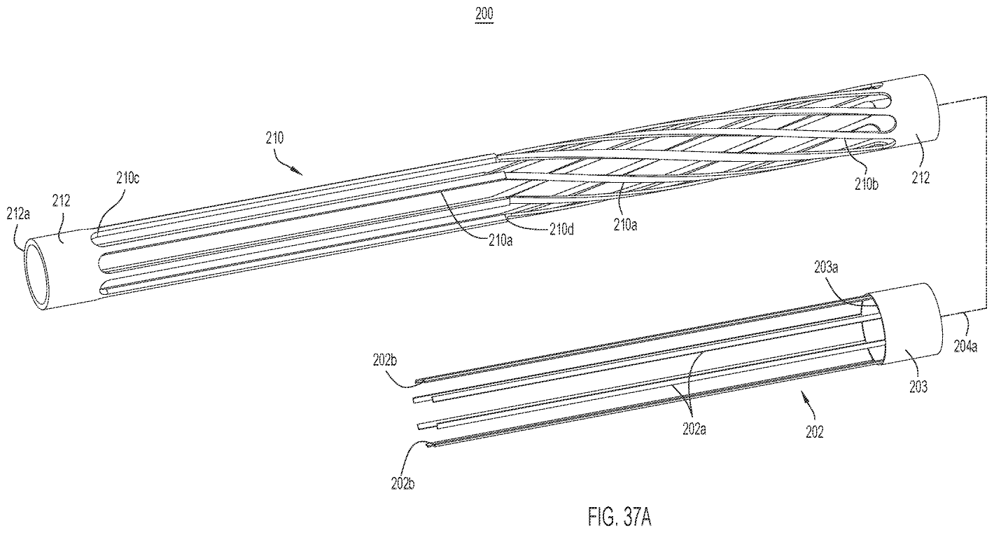

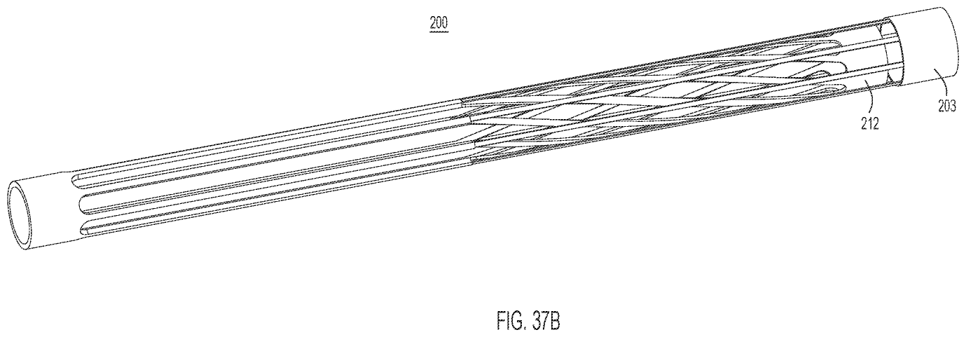

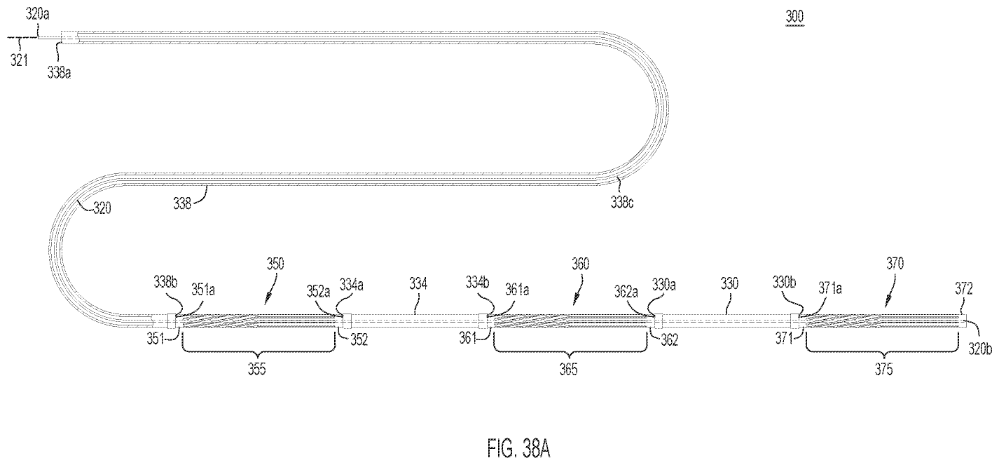

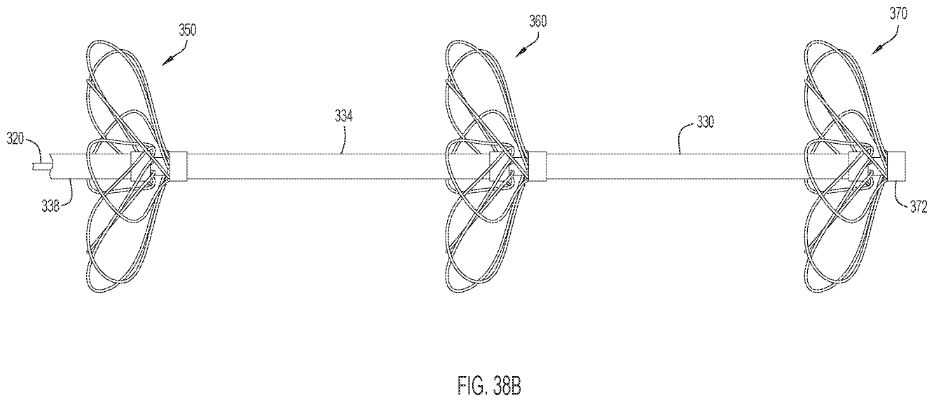

FIGS. 37A and 37B depict a clot/obstruction retrieval device assembly 200 that includes a reinforcement apparatus 202 having struts 202a that are coupled to a midsection of a retrieval device 210 for the purpose of inhibiting a reverse inversion of the elongate clot capturing elements 210a of the retrieval device as the retrieval device is advanced proximally across a clot/obstruction. Any of the retrieval devices 12, 150 and 160 disclosed above and any of the retrieval devices disclosed below may be fitted with a similar reinforcement apparatus to achieve the same objective.

Each of the reinforcement apparatus 202 and retrieval device 210 may be laser cut from a tube in order to achieve the configurations shown in FIG. 37A. Like retrieval devices 12, 150 and 160 disclosed above, the retrieval device 210 need only comprise elongate clot capturing elements that extend from a proximal collar to a distal collar of the device. In many of the example implementations shown in the figures herein, the elongate clot capturing elements have a proximal section and a distal section with the proximal sections arranged in a helical pattern about a longitudinal axis of the retrieval device and the distal sections being arranged parallel to the longitudinal axis. However, as explained above, none of the retrieval devices disclosed herein is limited to such a configuration. Other configurations may include, in an unlimited way, to the examples of FIGS. 8A-B, 8E, 9B, 10A-B, 12, 13A-B, 14A-B, 15A-C, 16A-B, 17A-B and 18A-B of U.S. Pat. No. 10,555,745.

In the implementation of FIGS. 37A and 37B the reinforcement apparatus 202 includes a proximal collar 203 from which distally extends therefrom a plurality of straight reinforcing struts 202a that are each arranged parallel to the longitudinal axis 204a of the proximal collar. (It is important to note that the struts 202a need not be straight and may comprise any shape and orientation capable of inhibiting a backward inversion of the clot capturing elements 210 of retrieval device 202.) Each of the reinforcing struts 202a has a distal end 202b that is each attached to a midsection of one of the elongate clot capturing elements 210a of retrieval device 210 as shown in FIG. 37B. (The “midsection” meaning a location closer to a middle 210d of the elongate clot capturing elements 210a than either of the proximal end 210b and distal end 210c.) The distal ends 202b of struts 202a may be attached to the elongate clot capturing elements 210a by any of a variety of methods, including by use of an adhesive, by soldering, by welding, etc. In operation when the retrieval device 210 is in a fully expanded state, the reinforcing struts 202 buttress a midsection of the elongate clot capturing elements 210a to inhibit a backward inversion of the clot capturing elements.

In the implementation of FIGS. 37A and 37B the number of reinforcing struts 202a and elongate clot capturing elements 210a is the same. According to other implementations there are fewer reinforcing struts 202 than there are elongate clot capturing elements 210a. For example, according to one implementation the retrieval device 210 includes eight elongate clot capturing elements 210a and the reinforcement apparatus 202 includes four reinforcement struts 202a. According to other implementations the retrieval device 210 includes six elongate clot capturing elements 210a and the reinforcement apparatus 202 includes three reinforcement struts 202a. In each of these implementations it is preferable that the reinforcing struts 202a be attached symmetrically to every other elongate clot capturing element 210a.

In the preceding disclosure clot/obstruction retrieval assemblies have been presented in which they include a single retrieval device 12 or two retrieval devices 150 and 160. It is important to emphasize that the invention is not limited to clot/obstruction retrieval devices having any particular number of retrieval devices. That is, they may include three, four or more retrieval devices arranged in series and deployed to their expanded states in a manner similar to what has been described above.

FIG. 38A illustrates an assembly 300 useful in removing obstructions (e.g. blood clots) located within a bodily duct of a patient. The assembly 300 includes first retrieval device 350, a second retrieval device 360 and a third retrieval device 370 that are each capable of transitioning between an unexpanded state as shown in FIG. 38A and an expanded state as shown in FIG. 38B. As shown in the FIG. 38B, the expanded state may be a fully expanded state. The third retrieval device 370 is disposed distal to the second retrieval device 360 and the second retrieval device 360 is disposed distal to the first retrieval device 350. In the implementation shown in FIG. 38A, each of retrieval devices 350, 360 and 370 may have the same configuration as the retrieval device 12 shown in FIG. 1C. In the implementation shown in FIG. 38B, each of retrieval devices 350, 360, 370 may have the same configuration as the retrieval device 12 shown in FIGS. 1D and 1E. However, it is important to note that retrieval devices 350, 360, 370 may comprise other configurations such as the configurations of retrieval devices 12 disclosed above and the retrieval device configurations disclosed in U.S. Pat. No. 10,555,745. Moreover, as will be discussed in more detail below, the configuration of each the first, second and third retrieval devices may differ from one or both of the other retrieval devices in one or both of its nonexpanded and expanded configurations.

The assembly 300 includes an elongate wire 320 having a proximal end 320a, a distal end 320b, and a longitudinal axis 321 on which is mounted the first, second and third retrieval devices 350, 360 and 370. The first retrieval device 350 includes a proximal collar 351 and a distal collar 352 that each possesses a longitudinal through opening through which the elongate wire 320 passes. Disposed between the proximal collar 351 and distal collar 352 is a plurality of elongate clot capturing elements 355 that each has a proximal end attached to the proximal collar 351 and a distal end attached to the distal collar 352. The second retrieval device 360 includes a proximal collar 361 and a distal collar 362 that each possesses a longitudinal through opening through which the elongate wire 320 passes. Disposed between the proximal collar 361 and distal collar 362 is a plurality of circumferentially spaced-apart elongate clot capturing elements 365 that each has a proximal end attached to the proximal collar 361 and a distal end attached to the distal collar 362. The third retrieval device 370 includes a proximal collar 371 and a distal collar 372 that each possesses a longitudinal through opening through which the elongate wire 320 passes. Disposed between the proximal collar 371 and distal collar 372 is a plurality of circumferentially spaced-apart elongate clot capturing elements 375 that each has a proximal end attached to the proximal collar 371 and a distal end attached to the distal collar 372. The elongate clot capturing elements 355, 365 and 375 may take on a variety of different configurations such as, for example, those disclosed in conjunction with the elongate clot capturing elements 15, 155 and 165 previously disclosed.

The distal collar 372 of the third retrieval device is operatively coupled with the elongate wire 320. In the implementation of FIGS. 38A and 38B, the distal collar 372 is fixed to the distal end 320b or distal end portion of the elongate wire 320. The distal collar 372 may be fixed to the elongate wire using an adhesive, by soldering, by welding, etc. In an alternative implementation, the distal collar 372 is not fixed to the elongate wire 320. In such implementations a stop (like stops 25 and 125 disclosed above) is provided on the elongate wire 320. The stop includes a proximal facing surface that faces a distal facing surface of the distal collar 372. The stop may be formed integrally with the wire 320 or may be secured to the wire with the use of an adhesive, or by other fastening methods, such as, by soldering, welding, etc.

Disposed between the distal collar 362 of the second retrieval device 360 and the proximal collar 371 of the third retrieval device 370 is a first tube 330 having a through passage through which the elongate wire 320 passes. The first tube 330 has a proximal end 330a that interfaces a distal end 362a of the second retrieval device 360. The first tube 330 also has a distal end 330b that interfaces a proximal end 371a of the third retrieval device 370.

Disposed between the distal collar 352 of the first retrieval device 350 and the proximal collar 361 of the second retrieval device 360 is a second tube 334 having a through passage through which the elongate wire 320 passes. The second tube 334 has a proximal end 334a that interfaces a distal end 352a of the first retrieval device 350. The second tube 334 also has a distal end 334b that interfaces a proximal end 361a of the second retrieval device 360.

The assembly 300 also includes a third tube 338 mounted on the elongate wire 320. The third tube 338 has a proximal end 338a, a distal end 338b, and a longitudinal through opening/passageway 338c through which the elongate wire 320 passes. According to some implementations, the proximal end 351a of the first retrieval device 350 interfaces the distal end 338b of the third tube 338.

According to some implementations, the assembly 300 is configured such that each of the first, second and third retrieval devices 350, 360 and 370 transitions from the unexpanded state to the expanded state at least partially by a withdrawing of the elongate wire 320 proximally (in a direction towards the medical practitioner) while the tube 338 is held stationary. The first, second and third retrieval devices 350, 360 370, elongate wire 320 and first, second and third tubes 330, 334, 338 are assembled such that as the elongate wire 320 is withdrawn proximally, the longitudinal distance between the proximal collar 351, 361, 371 and distal collar 352, 362, 372 of the respective first, second and third retrieval devices 350, 360, 370 decreases to induce a bending of the plurality of elongate clot capturing elements 355, 365, 375 as shown in FIG. 38B. As noted above, according to some implementations each of the plurality of elongate clot capturing elements 355, 365, 375 may be configured to bend to form petal-like structures in which adjacent petal-like structures at least partially overlap with one another. That is, each of the first, second and third plurality of elongate clot capturing elements 355, 365, 375 may have the same or similar configuration of the plurality of elongate clot capturing elements 15 shown in FIG. 1E.

According to some implementations, the assembly 300 is also configured such that when the first, second and third retrieval devices 350, 360, 370 are in the expanded state, they are capable of transitioning from the expanded state towards or to the unexpanded state by a pushing of the elongate wire 320 distally (in a direction away from the medical practitioner). According to such implementations, the first, second and third retrieval devices 350 360, 370 elongate wire 320 and first, second and third tubes 330, 334, 338 are assembled such that as the elongate wire 320 is pushed distally, the distance between the proximal collar 351, 361, 371 and distal collar 352, 362, 372 of the respective first, second and third retrieval devices 350, 360, 370 increases.

According to some implementations, the assembly 300 is configured such that each of the first, second and third retrieval devices 350, 360, 370 transitions from the unexpanded state to the expanded state at least partially by a pushing of the tube 338 distally (in a direction away from the medical practitioner) while the elongate wire 320 is held stationary. According to such implementations, the first, second and third retrieval devices 350, 360, 370, elongate wire 320 and first, second and third tubes 330. 334, 338 are assembled such that as the tube 338 is pushed distally, the proximal collar 351, 361, 371 moves distally so that the axial/longitudinal distance between the proximal collar 351 and distal collar 352 decreases to induce a bending of the plurality of elongate clot capturing elements 355, so that the axial/longitudinal distance between the proximal collar 361 and distal collar 362 decreases to induce a bending of the plurality of elongate clot capturing elements 365 and so that the axial/longitudinal distance between the proximal collar 371 and distal collar 372 decreases to induce a bending of the plurality of elongate clot capturing elements 375.

The assembly 300 may further include an actuation device like the actuation devices 40 and 140 disclosed above, such that an end or end section of one of the elongate wire 320 and third tube 338 is attached/coupled to the sliding member 42, 142 and an end or end section of the other is attached coupled to a fixed part (e.g. housing 41, 141) of the actuation device.

The manner in which the first, second and third retrieval devices 350, 360, 370 and first, second and third tubes 330, 334, 338 interface one another can vary.

According to one implementation, the first, second and third retrieval devices 350, 360, 370 and first and second tubes 330 and 334 are a unitary structure being made from a single piece of material. According to such an implementation, the distal end 338b of the third tube 338 may or may not be attached to the proximal end 351 of the first retrieval device 350.

According to other implementations, the proximal end 351 of the first retrieval device 350 may or may not be attached to the distal end 338b of the third tube 338, the distal end 352a of the first retrieval device 350 may or may not be attached to the proximal end 334a of the second tube 334, the distal end 334b of the second tube 334 may or may not be attached to the proximal end 361a of the second retrieval device 360, the distal end of the second retrieval device may or may not be attached to the proximal end 330a of the first the proximal end 371a, and the distal end 330b of the first tube 330 may or may not be attached to proximal end 371a of the third retrieval device 370.

According to one particular implementation, none of the first, second and third retrieval devices 350, 360, 370 and first, second and third tubes 330, 334, 338 are attached to one another and they interact with one another by their adjacent ends abutting one another.

With continued reference to FIGS. 38A and 38B, in use the first, second and third retrieval devices 350, 360, 370 while in their unexpanded states are delivered to a site within a bodily duct of a patient to a location downstream the clot/obstruction to be retrieved. Thereafter, the retrieval devices are deployed to their fully expanded states. Upon the retrieval devices 350, 360, 370 being deployed, they are withdrawn proximally for the purpose of capturing the clot/obstruction and removing it from the duct of the patient. A benefit of utilizing multiple retrieval devices disposed in series one after the other is that fewer clot/obstruction retrieval attempts are necessary for the satisfactorily removal of the clot/obstructions from the duct of the patient. In the example of FIG. 38B, the first (most proximal) retrieval device 350 first engages the clot/obstruction and any part of the clot/obstruction not captured by the first retrieval device may be fully or partially captured by the second retrieval device 360. Similarly, any part of the clot/obstruction not captured by the second retrieval device 360 may be fully or partially captured by the third retrieval device 370. As discussed above and further below, the configuration of the retrieval devices may differ to achieve an aggregate clot capturing capability tuned to achieve a desired result optimized to the location and type of obstruction to be retrieved.

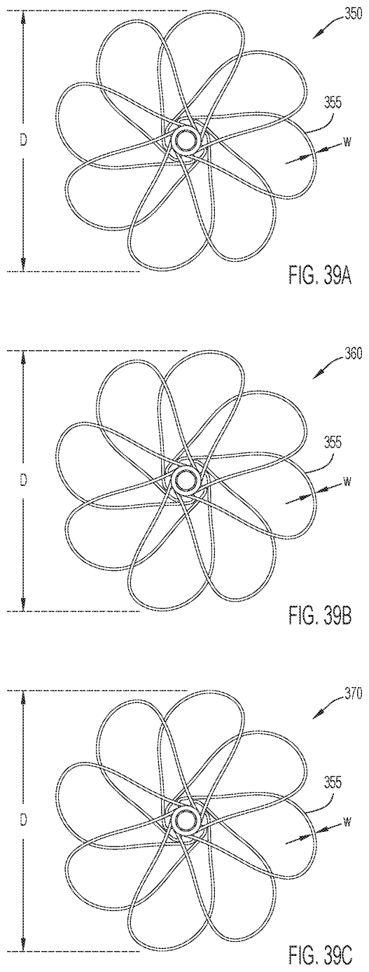

FIGS. 39A-C shows a front view of each of the first, second and third retrieval devices 350, 360 and 370 in their fully expanded state according to one implementation in which each of the retrieval devices has a same diameter “D” and the elongate clot capturing elements 355, 365 and 375 have a same width dimension “w”. According to one such implementation, the elongate clot capturing elements 355, 365 and 375 also have the same thickness dimension.

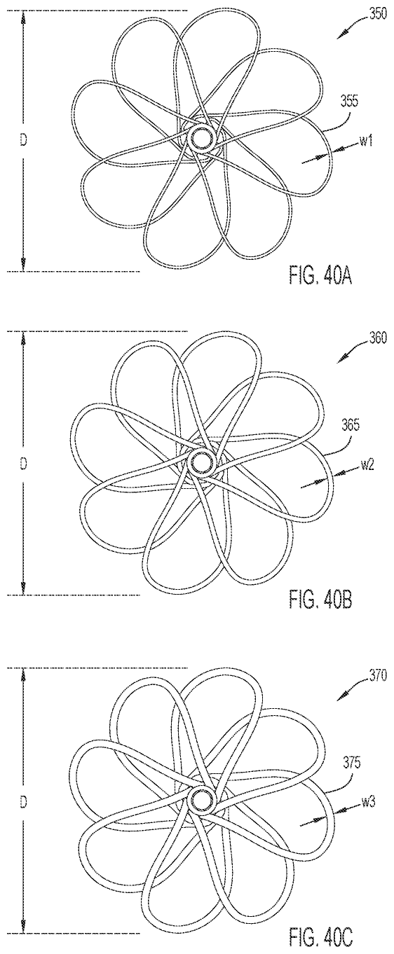

FIGS. 40A-C show a front view of each of the first, second and third retrieval devices 350, 360 and 370 in their fully expanded state according to another implementation in which each of the retrieval devices has a same diameter “D” and the elongate clot capturing elements 355, 365 and 375 have different cross-sectional areas. The different cross-sectional areas may be achieved by varying the width and/or thickness of the elongate clot capturing elements. In the implementation of FIGS. 40A-C the elongate clot capturing elements 355, 365, 375 have different widths with the width “w3” of elements 375 being greater than the width “w2” of elements 365, and with the width “w2” being greater than the width “w1” of elements 355. By varying the cross-sectional area of the elongate clot capturing elements among the first, second and third retrieval devices, the elongate clot capturing elements of each retrieval device may possess a different bending stiffness than the others. This can result in the first, second and third retrieval devices 350, 360, 370 being progressively more aggressive in its retrieval of the clot/obstruction. This can beneficially minimize the overall trauma to the duct wall of the patient during the retrieval process.

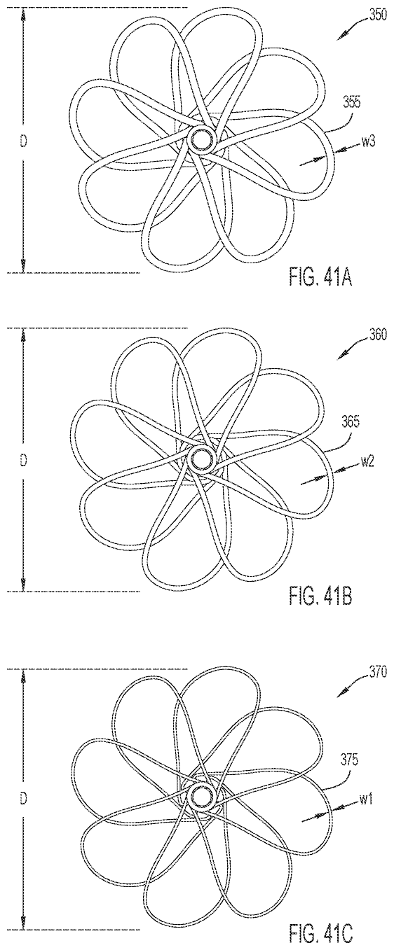

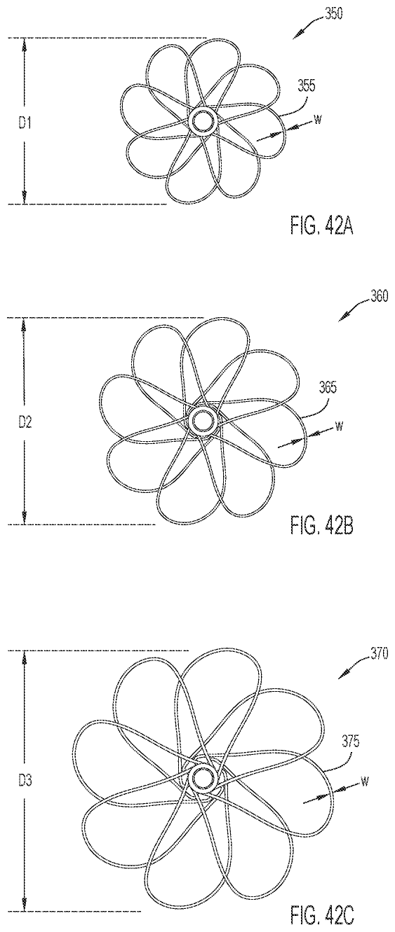

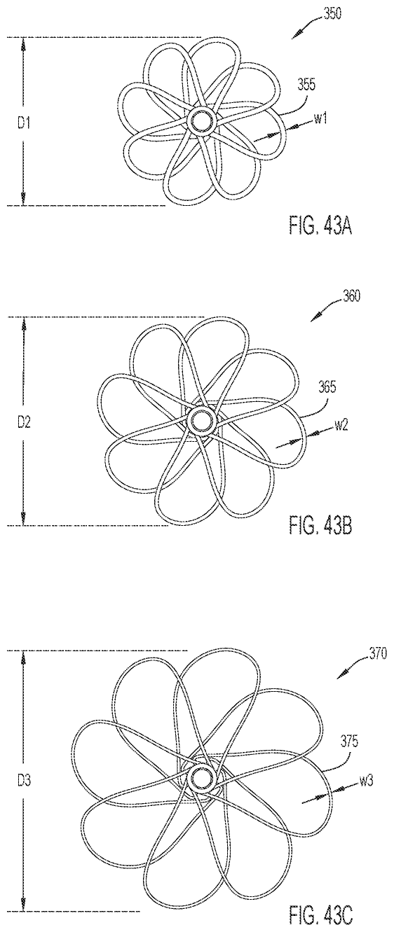

FIGS. 41A-C show a front view of each of the first, second and third retrieval devices 350, 360 and 370 in their fully expanded state according to another implementation in which each of the retrieval devices has a same diameter “D” and the elongate clot capturing elements 355, 365 and 375 have different cross-sectional areas. The different cross-sectional areas may be achieved by varying the width and/or thickness of the elongate clot capturing elements. In the implementation of FIGS. 41A-C the elongate clot capturing elements 355, 365, 375 have different widths with the width “w3” of elements 355 being greater than the width “w2” of elements 365, and with the width “w2” being greater than the width “w1” of elements 375. As explained above, varying the cross-sectional area of the elongate clot capturing elements among the first, second and third retrieval devices, the elongate clot capturing elements of each retrieval device may possess a different bending stiffness than the others. In the implementation of FIGS. 41A-C, this can result in the first, second and third retrieval devices 350, 360, 370 being progressively less aggressive in its retrieval of the clot/obstruction. This can beneficially minimize the overall trauma to the duct wall of the patient during the retrieval process.

FIGS. 42A-C show a front view of each of the first, second and third retrieval devices 350, 360 and 370 in their fully expanded state according to another implementation in which the first, second and third retrieval devices 350, 360, 370 respectively have different diameters “D1”, “D2”, “D3” (with D1<D2<D3) and the elongate clot capturing elements 355, 365 and 375 have the same cross-sectional area (which in some instances means the elongate clot capturing elements 355, 365, 375 have the same width “w” and a same thickness). An advantage of the configuration of FIGS. 42A-C is that it allows a sequential and gradual capturing of the clot/obstruction that can beneficially minimize the overall trauma to the duct wall of the patient. That is, instead of attempting to capture and remove an entirety of the clot/obstruction at a given time with a single retrieval device, the retrieval device configuration of FIGS. 42A-C can be utilized to perform a stepped and gradual retrieval of the clot/obstruction with the first retrieval device 350 capturing a first part of the clot/obstruction, the second retrieval device 360 thereafter capturing a second part of the clot obstruction and the third retrieval device 370 thereafter capturing any remaining part of the clot/obstruction. This can beneficially minimize the overall trauma to the duct wall of the patient during the retrieval process.

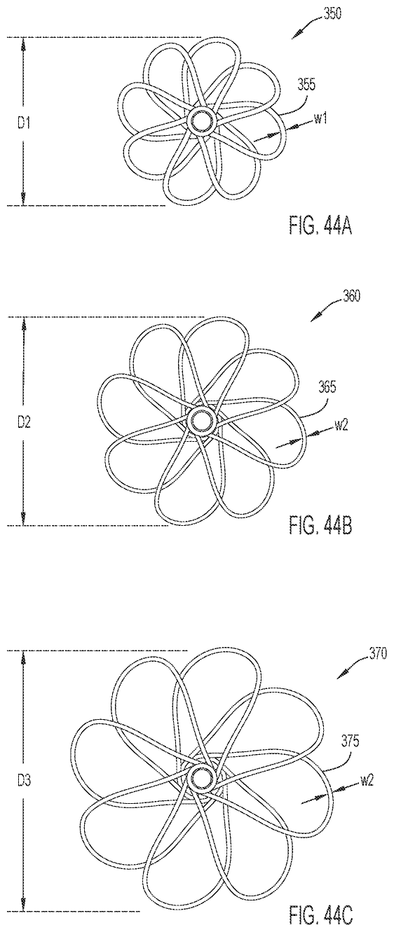

FIGS. 43A-C show a front view of each of the first, second and third retrieval devices 350, 360 and 370 in their fully expanded state according to another implementation in which the first, second and third retrieval devices 350, 360, 370 respectively have different diameters “D1”, “D2”, “D3” (with D1<D2<D3) and the elongate clot capturing elements 355, 365 and 375 have different cross-sectional areas (which in some instances means the elongate clot capturing elements 350, 360, and 370 respectively have widths “w1”, “w2” and “w3” with w1>w2>w3). An advantage of the configuration of FIGS. 43A-C is that it allows a sequential and gradual capturing of the clot/obstruction that can beneficially minimize the overall trauma to the duct wall of the patient. That is, instead of attempting to capture and remove an entirety of the clot/obstruction at a given time with a single retrieval device, the retrieval device configuration of FIGS. 43A-C can be utilized to perform a stepped and gradual retrieval of the clot/obstruction with the first retrieval device 350 capturing a first part of the clot/obstruction, the second retrieval device 360 thereafter capturing a second part of the clot obstruction and the third retrieval device 370 thereafter capturing any remaining part of the clot/obstruction. Another advantage is that (by virtues of their different cross-sectional areas), the elongate clot capturing elements 355 have a higher bending stiffness than the elongate clot capturing elements 360, which in turn has a higher bending stiffness than elongate clot capturing elements 370. In this way, the smaller diameter retrieval device 350 with the greatest bending stiffness is initially used to aggressively dislodge and capture at least a portion of the obstruction. This is followed by dislodging and capturing at least a portion of any remaining clot/obstruction in a less aggressive way by use of retrieval device 360. This is followed by dislodging and capturing at least a portion of any remaining clot/obstruction in even a less aggressive way by use of retrieval device 370. This can beneficially minimize the overall trauma to the duct wall of the patient.

FIGS. 44A-C show a front view of each of the first, second and third retrieval devices 350, 360 and 370 in their fully expanded state according to another implementation in which the first, second and third retrieval devices 350, 360, 370 respectively have different diameters “D1”, “D2”, “D3” (with D1<D2<D3) and the elongate clot capturing elements 355 have a different cross-sectional area than that of elongate clot capturing elements 365 and 375 (which in some instances means the elongate clot capturing elements 350 have a width “w1” that is greater than the width “w2” of clot capturing elements 360 and 370). An advantage of the configuration of FIGS. 44A-C is that it allows a sequential and gradual capturing of the clot/obstruction that can beneficially minimize the overall trauma to the duct wall of the patient. That is, instead of attempting to capture and remove an entirety of the clot/obstruction at a given time with a single retrieval device, the retrieval device configuration of FIGS. 44A-C can be utilized to perform a stepped and gradual retrieval of the clot/obstruction with the first retrieval device 350 capturing a first part of the clot/obstruction, the second retrieval device 360 thereafter capturing a second part of the clot obstruction and the third retrieval device 370 thereafter capturing any remaining part of the clot/obstruction. Another advantage is that (by virtue of their different cross-sectional areas), the elongate clot capturing elements 355 have a higher bending stiffness than the elongate clot capturing elements 360 and 370. In this way, the smaller diameter retrieval device 350 with the greatest bending stiffness is initially used to aggressively dislodge and capture at least a portion of the obstruction. This is followed by dislodging and capturing at least a portion of any remaining clot/obstruction in a less aggressive way by use of retrieval devices 360 and 370.

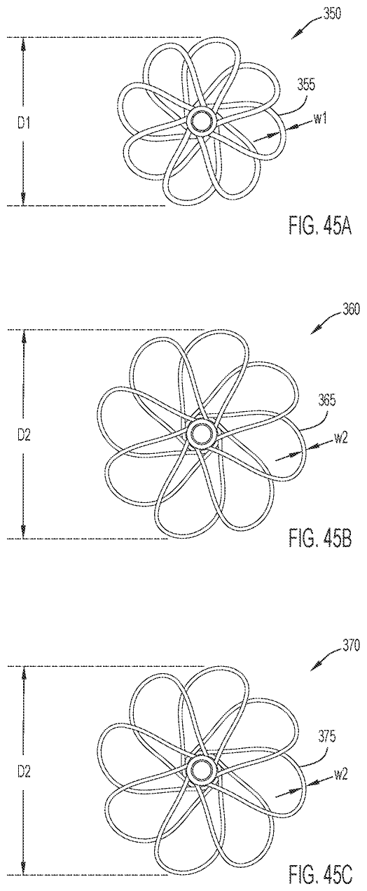

FIGS. 45A-C show a front view of each of the first, second and third retrieval devices 350, 360 and 370 in their fully expanded state according to another implementation in which the first retrieval device 350 has a diameter “D1” that is less than the diameter “D2” of each of retrieval devices 360 and 370. In addition, the elongate clot capturing elements 355 have a different cross-sectional area than that of elongate clot capturing elements 365 and 375 (which in some instances means the elongate clot capturing elements 350 have a width “w1” that is greater than the width “w2” of clot capturing elements 360 and 370). An advantage of the configuration of FIGS. 45A-C is that it allows a sequential and gradual capturing of the clot/obstruction that can beneficially minimize the overall trauma to the duct wall of the patient. That is, instead of attempting to capture and remove an entirety of the clot/obstruction at a given time with a single retrieval device, the retrieval device configuration of FIGS. 45A-C can be utilized to perform a stepped and gradual retrieval of the clot/obstruction with the first retrieval device 350 capturing a first part of the clot/obstruction, the second retrieval device 360 thereafter capturing a second part of the clot obstruction and the third retrieval device 370 thereafter capturing any remaining part of the clot/obstruction. Another advantage is that (by virtue of their different cross-sectional areas), the elongate clot capturing elements 355 have a higher bending stiffness than the elongate clot capturing elements 360 and 370. In this way, the smaller diameter retrieval device 350 with the greatest bending stiffness is initially used to aggressively dislodge and capture at least a portion of the obstruction. This is followed by dislodging and capturing at least a portion of any remaining clot/obstruction in a less aggressive way by use of retrieval devices 360 and 370.

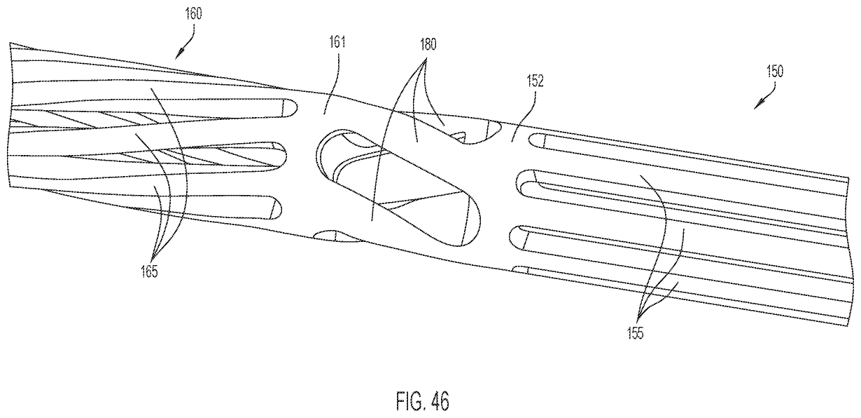

As disclosed above, in some instances the first retrieval device 150, second retrieval device 160 and the tube 130 disposed between them may be made from a single piece of material (e.g. a single Nitinol tube, a single tube of another shape memory alloy such as copper-aluminum-nickel, etc.) like that shown in FIG. 31. In the foregoing examples, the tubes between the retrieval devices are shown to have a solid wall construction with no openings in the circumferential wall. However, according to other implementations, like that shown in FIG. 46, a plurality of circumferentially spaced-apart struts 180 may be used to join the distal end of one retrieval device to the proximal end of another retrieval device. An advantage of using a plurality of struts 180 to join the retrieval devices in lieu of a solid tube 130 is that it provides the assembly with a greater degree of longitudinal flexibility, making it easier to maneuver the assembly through the tortuous anatomy of the patient. In the assembly of FIG. 46, the struts 180 are short enough to enable at least some of elongate clot capturing elements 165 of the second retrieval device 160 to abut the elongate clot capturing elements 155 of the first retrieval device 150 (like that shown in FIG. 30) when both retrieval devices are in their fully expanded state. However, according to other implementations the struts 180 are sufficiently long to prevent any part of the first and second retrieval devices from touching one another when they are both in their fully expanded state.

According to some implementations, the cross-sectional area of one or more or all of struts 180 is greater than the cross-sectional area of each of elongate clot capturing elements 155 and 165. This is to ensure that the struts 180 do not invert as the retrieval devices 150 and 160 transition from their unexpanded states to their fully expanded states. According to some implementations the one or more or all of struts 180 has/have a thickness and/or width that is greater than the thickness and/or width of the elongate clot capturing elements 155 and 165.

As shown in FIG. 46, according to some implementations struts 180 are each arranged non-parallel to the longitudinal axes 181 of the first and second retrieval devices 150 and 160. According to other implementations, struts 180 are each arranged parallel to the longitudinal axes 181 of the first and second retrieval devices 150 and 160.

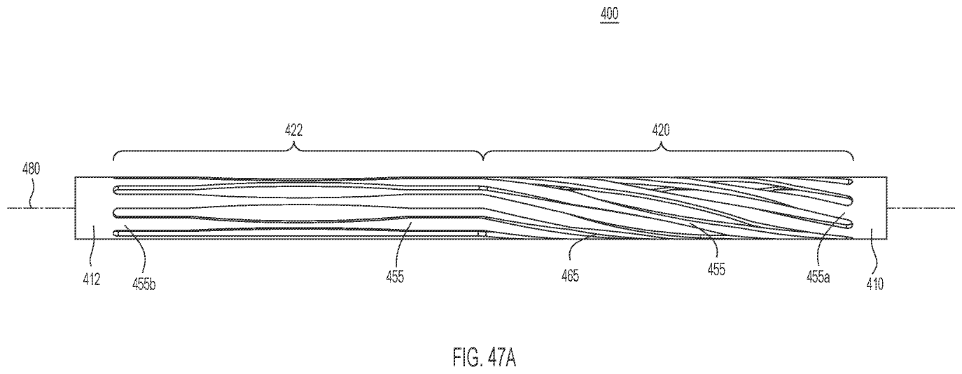

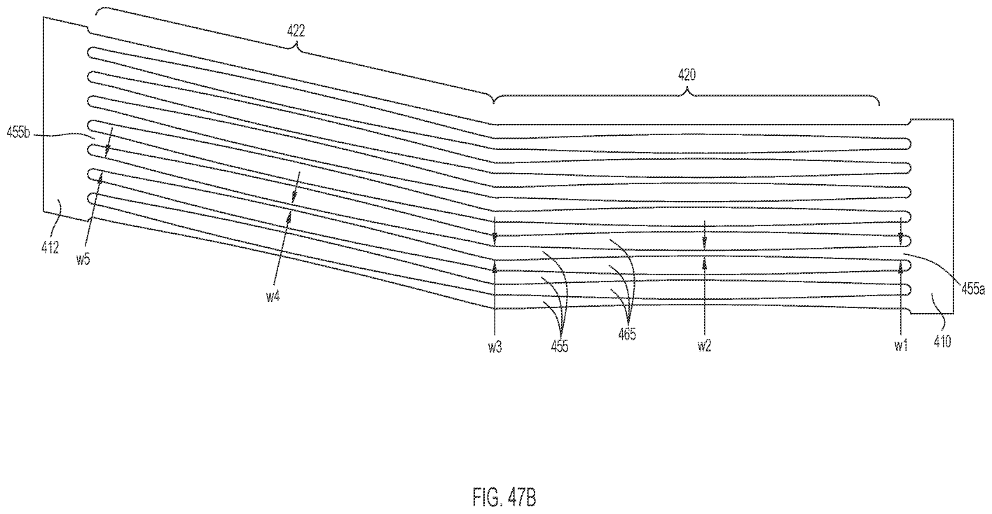

FIG. 47A shows a retrieval device 400 according to another implementation in which the elongate clot capturing elements 455 are selectively tapered so that the strain induced in them as they bend (as the retrieval device transitions between the unexpanded and expanded states) is more broadly distributed along their length. This beneficially enables higher forces to be applied to the elongate clot capturing elements 455 with less strain. This results in stronger elongate clot capturing element structures that are more durable when subject to repeated deflections.

With continued reference to FIG. 47A, the elongate clot capturing elements 455 have proximal and distal ends 455a and 455b that are respectively attached to the proximal and distal collars 410 and 412 of retrieval device 400. In the implantation of FIG. 47A, the elongate clot capturing elements 455 comprise a proximal end section 420 and a distal end section 422 with the proximal end section 420 of each of the elongate clot capturing elements 455 curving about a longitudinal axis of the retrieval device 400, and with the distal end section 422 of each of the elongate clot capturing elements 455 being straight and arranged parallel with the longitudinal axis 480 of the retrieval device 400 when the retrieval device is in the unexpanded state as shown in FIG. 47A. As shown in FIG. 47A, at a junction between the curved and straight portions of the elongate clot capturing elements 455, the width “w3” (see FIG. 47B) of each the elongate clot capturing elements is at a maximum.

FIG. 47B is a representation of the retrieval device of FIG. 47A showing an example in which the width of the elongate clot capturing elements 455 change along their length. The representation of FIG. 47B does not show the true arrangement of the elongate clot capturing elements 455 as shown FIG. 47A, and is provided only for the purpose of showing how the width of the elongate clot capturing elements change along their length. In the example of FIG. 47B the proximal ends 455a of the elongate clot capturing elements 455 have a first width dimension “w1”, the middle portions of the proximal end section 420 of the elongate clot capturing elements have a second width dimension “w2”, and the junctions where the proximal and distal end sections 420 and 422 of the elongate clot capturing elements meet have a third width dimension “w3”. Further, the distal ends 455b of the elongate clot capturing elements 455 have a fifth width dimension “w5” and the middle portions of the distal end section 420 of the elongate clot capturing elements 455 have a fourth width dimension “w4”.

According to some implementations w1, w3 and w5 are substantially the same, and w2 and w4 are substantially the same with each of w2 and w4 being less than each of w1, w3 and w5. According to other implementations w1 and w5 are not substantially the same as w3. “Substantially the same” meaning that their width dimensions do not deviate from one another by more than 15%. In cases where the retrieval device 400 is formed by laser cutting a tube to form the elongate clot capturing elements 455, the forestated width relationships may be the as-cut dimensions of the elongate clot capturing elements prior to them being electropolished or may be their dimensions after being electropolished.

In the example of FIGS. 47A and 47B, w1, w3 and w4 represent points of maximum width along the length of the elongate clot capturing elements, and w2 and w4 represent points of minimum width along the length of the elongate clot capturing elements. According to some implementations, the point of minimum width w2 is located at a midpoint between w1 and w3, and the point of minimum width w4 is located at a midpoint between w3 and w5. Between the locations of w1 and w2 the elongate clot capturing elements 455 gradually diminish in width and between the locations of w2 and w3 the elongate clot capturing elements gradually increase in width. Between the locations of w3 and w2 the elongate clot capturing elements 455 gradually diminish in width and between the locations of w4 and w5 the elongate clot capturing elements gradually increase in width. Preferably each side of the elongate clot capturing elements is tapered to achieve the reductions and increases in width. According to some implementations, the ratio of w3/w2 and the ratio w3/w4 is 1.3 to 1.9.

In the implementation of FIGS. 47A and 47B, neighboring elongate clot capturing elements 455 are circumferentially separated from one another by an elongate slot 465 that extends continuously between the proximal collar 410 and distal collar 412. In the implementation of FIGS. 47A and 47B all of the elongate clot capturing elements comprise a tapered configuration. However, according to other implementations not all of the elongate clot capturing elements comprise tapered proximal and distal end sections. For example, according to some implementations every other of the elongate clot capturing elements comprise the forestated tapered configurations.

According to one implementation, the post electropolishing dimensions of retrieval device 400 are: w1=0.0038 inches, w2=0.0026 inches, w3=0.0042 inches, w4=0.0026 inches, w5=0.0043 inches, the outer diameter of the retrieval device in its unexpanded state as shown in FIG. 47A being 0.0792 inches, and the wall thickness of the retrieval device being 0.0047 inches.

Removing a clot/obstruction from an arterial passageway of a patient typically includes the delivery of a guidewire across the clot/obstruction and a subsequent advancement of a delivery catheter over the guidewire so that a distal end portion of the delivery catheter resides distal to the clot/obstruction. With the delivery catheter in place, the one or more retrieval devices is/are loaded into the delivery catheter. According to some implementations, the one or more retrieval devices is/are then carried to the location distal to the clot/obstruction and is/are subsequently deployed from the distal end of the delivery catheter into the arterial passageway of the patient. The one or more retrieval devices may be deployed out the distal end of the delivery catheter by either withdrawing the delivery catheter in a proximal direction while holding the one or more retrieval devices in place or by holding the delivery catheter fixed while distally advancing the one or more retrieval devices.

When in the deployed state inside the passageway of the patient, the looped elongate clot capturing elements of the one or more retrieval devices press against the arterial wall of the vessel. After the one or more retrieval devices is/are deployed inside the passageway, it/they are ready to be withdrawn proximally to engage/capture the clot/obstruction. Upon the obstruction/clot being captured by the one or more retrieval devices, removal of the obstruction/clot may be accomplished, at least in part, by withdrawing the one or more retrieval devices to a mouth of an aspiration catheter or into the delivery catheter.

In some implementations the elongate clot capturing elements of the retrieval devices disclosed herein are configured to sweep along the arterial wall to which the clot/obstruction is attached during the removal process to cause the clot/obstruction, or remnants of the clot/obstruction, to be moved centrally towards the center of the affected vessels.

In the following clauses additional implementations are disclosed.

### Group a Clauses:

Clause 1. A clot retrieval assembly comprising:
an elongate wire having a proximal end, a distal end, and a longitudinal axis.
a retrieval device comprising:
   a distal collar operatively coupled with the elongate wire;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section;
   the retrieval device being configured to transition from an unexpanded state to a fully expanded state, in the unexpanded state no portion of the plurality of elongate clot capturing elements is located over the proximal collar, in the fully expanded state at least portions of the plurality of elongate clot capturing elements are inverted and residing over the proximal collar; and
a tube mounted on the elongate wire and having a longitudinal through opening through which the elongate wire passes, the proximal collar being attached to a distal end of the tube or otherwise arranged such that the proximal facing surface of the proximal collar faces a distal facing surface of the tube without the proximal collar being attached to the tube;
the retrieval device being configured to transition towards or to a fully expanded state at least partially by withdrawing the elongate wire proximally while the tube is held stationary.

Clause 2. The clot capturing assembly according to clause 1, wherein the retrieval device, elongate wire and tube are assembled such that as the elongate wire is withdrawn proximally a distance between the proximal and distal collars decreases to induce a bending of the plurality of elongate clot capturing elements.

Clause 3. The clot retrieval assembly according to clause 1, wherein the distal collar is attached to the elongate wire.

Clause 4. The clot retrieval assembly according to clause 1, wherein the distal collar is not attached to the elongate wire.

Clause 5. The clot retrieval assembly according to clause 4, further comprising a stop on the distal end or distal end portion of the elongate wire, the stop including a proximal facing surface that faces a distal facing surface of the distal collar, the proximal facing surface of the stop being configured to press against the distal facing surface of the distal collar upon the elongate wire being withdrawn proximally to cause or assist in causing the retrieval device to transition towards or to the fully expanded state.

Clause 6. The clot retrieval assembly according to clause 1, wherein the proximal collar, distal collar and the plurality of elongate clot capturing elements are made from a single piece of material.

Clause 7. The clot retrieval assembly according to clause 6, wherein the plurality of elongate clot capturing elements are laser cut from a tubular member.

Clause 8. The clot retrieval assembly according to clause 1, wherein a width of one or more of the plurality of elongate clot capturing elements varies along a length thereof.

Clause 9. The clot retrieval device according to clause 8, wherein when the retrieval device is in the fully expanded state each of the plurality of elongate clot capturing elements forms a loop having an apex portion, the apex portion of one or more of the elongate clot capturing elements having a reduced width compared to a remainder of the one or more elongate clot capturing elements.

Clause 10. The clot retrieval assembly according to clause 1, wherein the distal end section of one or more of the plurality of elongate clot capturing elements has a cross-sectional area greater than the cross-sectional area of the respective one or more proximal end section of the one or more of the plurality of elongate clot capturing elements.

Clause 11. The clot retrieval assembly according to clause 1, wherein the distal end section of one or more of the plurality of elongate clot capturing elements has a thickness greater than a thickness of the respective one or more proximal end section of the one or more of the plurality of elongate clot capturing elements.

Clause 12. The clot retrieval assembly according to clause 1, wherein the distal end section of one or more of the plurality of elongate clot capturing elements has a width greater than a width of the respective one or more proximal end section of the one or more of the plurality of elongate clot capturing elements.

Clause 13. The clot retrieval assembly according to clause 1, wherein the distal end section of each of the plurality of elongate clot capturing elements has a thickness greater than a thickness of the respective proximal end section of the plurality of elongate clot capturing elements.

Clause 14. The clot retrieval assembly according to clause 1, wherein the distal end section of each of the plurality of elongate clot capturing elements has a width greater than a width of the respective proximal end section of the plurality of elongate clot capturing elements.

Clause 15. The clot retrieval assembly according to clause 1, further comprising an actuation device having a moveable first part to which a proximal end or proximal end portion of the elongate wire is coupled, the actuation device having a stationary second part to which the proximal end or proximal end portion of the tube is coupled.

Clause 16. The clot retrieval assembly according to clause 6, wherein the proximal end section of each of the plurality of elongate clot capturing elements is arranged helically with respect to a longitudinal axis of the retrieval device.

Clause 17. The clot retrieval assembly according to clause 1, wherein the plurality of elongate clot capturing elements are configured to self-expand to place the retrieval device in at least a partially expanded state.

Clause 18. The clot retrieval assembly according to clause 17, further comprising a sheath in which the retrieval device is housed for maintaining the clot retrieval device in the unexpanded state.

Clause 19. The clot retrieval assembly according to clause 17, further comprising a delivery catheter in which the retrieval device and at least a portion of each of the elongate wire and tube are housed, the delivery catheter being configured to deliver the retrieval device through a vasculature of a patient towards a site of an obstruction.

Clause 20. The clot retrieval assembly according to clause 1, wherein none of the plurality of elongate clot capturing elements overlap with one another when the retrieval device is in the unexpanded state.

Clause 21. The clot retrieval assembly according to clause 20, wherein at least some of the plurality of elongate clot capturing elements overlap with one another when the retrieval device is in the fully expanded state.

Clause 22. The clot retrieval assembly according to clause 1, wherein the retrieval device is configured to transition from the fully expanded state towards the unexpanded state by pushing the elongate wire distally.

Clause 23. The clot retrieval device according to clause 1, wherein the tube is made of a metal, the tube including an annular wall comprising a plurality of through holes extending radially through the annular wall, the plurality of through holes being arranged to increase the longitudinal flexibility of the tube compared to the longitudinal flexibility of the tube absent the plurality of through holes.

### Group B Clauses:

Clause 1. A method for making a clot retrieval assembly, the method comprising:
laser cutting a tubular member to produce a retrieval device that includes a proximal collar, a distal collar, and a plurality of elongate clot capturing elements that each has a proximal end attached to the proximal collar and a distal end coupled to the distal collar, the tubular member being laser cut so that each pair of neighboring elongate clot capturing elements is circumferentially separated from one another by an elongate slot that extends continuously between the proximal and distal collars;
mounting a tube on an elongate wire, the elongate wire having a proximal end, a distal end, and a longitudinal axis, the tube having a longitudinal through opening through which the elongate wire passes;
mounting the retrieval device on the elongate wire at a location distal to the tube such that the elongate wire extends through the proximal and distal collars, the proximal collar being attached to a distal end of the tube or otherwise arranged such that a proximal facing surface of the proximal collar faces a distal facing surface of the tube without the proximal collar being attached to the tube; and
operatively coupling a distal end or a distal end portion of the elongate wire to the distal collar of the retrieval device;
the retrieval device, elongate wire and tube being assembled such that as the elongate wire is withdrawn proximally a distance between the proximal and distal collars decreases to cause a bending of the plurality of elongate clot capturing elements.

Clause 2. The method according to clause 1, wherein the tubular member has an annular wall, the method further comprising reducing a thickness of a portion of the annular wall prior to laser cutting the tubular member.

Clause 3. The method according to clause 2, wherein each of the plurality of elongate clot capturing elements has a proximal end section and a distal end section, the reduced thickness portion of the annular wall residing in the proximal end sections of the plurality of elongate clot capturing elements.

Clause 4. The method according to clause 1, wherein each of the plurality of elongate clot capturing elements has a proximal end section and a distal end section, the proximal end sections being laser cut to have a width less than a width of the distal end sections.

Clause 5. The method according to clause 1, wherein each of the plurality of elongate clot capturing elements includes a proximal end section and a distal end section, the method comprising laser cutting the tubular member so that the proximal end sections of the plurality of elongate clot capturing elements are arranged helically with respect to a longitudinal axis of the retrieval device.

Clause 6. The method according to clause 5, wherein the tubular member is laser cut so that the distal end sections of the plurality of elongate capture are arranged straight and parallel to the longitudinal axis of the retrieval device.

Clause 7. The method according to clause 1, wherein after the tubular member is laser cut to produce the retrieval device, the plurality of elongate clot capturing elements are shape-set while the retrieval device is in at least a partially expanded state.

Clause 8. The method according to clause 1, wherein after the tubular member is laser cut to produce the retrieval device, the plurality of elongate clot capturing elements are shape-set while the retrieval device is in the fully expanded state.

Clause 9. The method according to clause 7, wherein the tubular member is made of a shape memory alloy.

Clause 10. The method according to clause 8, wherein the tubular member is made of a shape memory alloy.

Clause 11. The method according to clause 9, further comprising constraining the retrieval device in a sheath to cause the retrieval device to assume a radially constrained unexpanded state.

### Group C Clauses:

Clause 1. A clot retrieval assembly comprising:
an elongate wire having a proximal end, a distal end, and a longitudinal axis;
a first retrieval device mounted on the elongate wire and comprising:
   a distal collar operatively coupled with the elongate wire;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
   the first retrieval device being configured to transition from an unexpanded state to a fully expanded state, in the unexpanded state no portion of the plurality of elongate clot capturing elements is located over the proximal collar, in the fully expanded state at least some of portions of the plurality of elongate clot capturing elements are inverted and residing over the proximal collar;
a second retrieval device mounted on the elongate wire at a location proximal to the first retrieval device, the second retrieval device comprising:
   a distal collar mounted on the elongate wire, the distal collar of the second retrieval device having a distal facing surface;
   a proximal collar mounted on the elongate wire and having a longitudinal through opening through which the elongate wire passes, the proximal collar of the second retrieval device having a proximal facing surface;
   a plurality of elongate clot capturing elements that each has a proximal end attached to the proximal collar of the second retrieval device and a distal end attached to the distal collar of the second retrieval device, each of the plurality of elongate capturing elements of the second retrieval device having a proximal end portion and a distal end portion, each pair of neighboring elongate clot capturing elements of the second retrieval device being circumferentially separated from one another by an elongate slot that extends continuously between the proximal and distal collars of the second retrieval devices;
   the second retrieval device being configured to transition from an unexpanded state to the fully expanded state, in the unexpanded state no portion of the plurality of elongate clot capturing elements of the second retrieval device is located over the proximal collar, in the fully expanded state at least some portions of the plurality of elongate clot capturing elements of the second retrieval device are inverted and residing over the proximal collar;
a first tube mounted on the elongate wire between the proximal collar of the first retrieval device and the distal collar of the second retrieval device, the first tube having a distal facing surface that faces the proximal facing surface of the proximal collar of the first retrieval device, the first tube having a proximal facing surface that faces the distal facing surface of the distal collar of the second retrieval device; and
a second tube mounted on the elongate wire and having a longitudinal through opening through which the elongate wire passes, the proximal collar of the second retrieval device being attached to a distal end of the second tube or otherwise arranged such that the proximal facing surface of the proximal collar of the second retrieval device faces a distal facing surface of the second tube without the proximal collar being attached to the second tube;
the first and second retrieval devices being configured to transition from their respective unexpanded states towards or to their respective fully expanded states at least partially by withdrawing the elongate wire proximally while the second tube is held stationary.

Clause 2. The clot retrieval assembly according to clause 1, wherein the first and second retrieval devices, elongate wire and first and second tubes are assembled such that as the elongate wire is withdrawn proximally a first distance between the proximal and distal collars of the first retrieval device decreases to induce a bending of the plurality of elongate clot capturing elements of the first retrieval device and a second distance between the proximal and distal collars of the second retrieval device decreases to induce a bending of the plurality of elongate clot capturing elements of the second retrieval device.

Clause 3. The clot retrieval assembly according to clause 1, wherein the distal collar of the first retrieval device is attached to the elongate wire.

Clause 4. The clot retrieval assembly according to clause 1, wherein the distal collar of the first retrieval device is not attached to the elongate wire.

Clause 5. The clot retrieval assembly according to clause 4, further comprising a stop on the distal end or distal end portion of the elongate wire, the stop including a proximal facing surface that faces a distal facing surface of the distal collar of the first retrieval device, the proximal facing surface of the stop being configured to press against the distal facing surface of the distal collar of the first retrieval device upon the elongate wire being withdrawn proximally to cause, or assist in causing, the first retrieval device to transition from the unexpanded state to the expanded state.

Clause 6. The clot retrieval assembly according to clause 5, wherein upon the elongate wire being withdrawn proximally, the proximal facing surface of the proximal collar of the first retrieval device presses against the distal facing surface of the first tube and the proximal facing surface of the first tube presses against the distal facing surface of the distal collar of the second retrieval device to cause, or assist in causing, the second retrieval device to transition from the unexpanded state towards or to the fully expanded state.

Clause 7. The clot retrieval assembly according to clause 1, wherein the proximal collar, distal collar and the plurality of elongate clot capturing elements of the first retrieval device are made from a first single piece of material and the proximal collar, distal collar and the plurality of elongate clot capturing elements of the second retrieval device are made from a second single piece of material.

Clause 8. The clot retrieval assembly according to clause 7, wherein the plurality of elongate clot capturing elements of the first retrieval device are laser cut from a first metal tubular member and the plurality of elongate clot capturing elements of the second retrieval device are laser cut from a second metal tubular member.

Clause 9. The clot retrieval assembly according to clause 1, wherein the plurality of elongate clot capturing elements of the first and second retrieval device are laser cut from a same tubular member.

Clause 10. The clot retrieval assembly according to clause 1, wherein the first retrieval device, second retrieval device and first tube are made from a single piece of material.

Clause 11. The clot retrieval assembly according to clause 1, wherein the distal end sections of the plurality of elongate clot capturing elements of the first retrieval device have a thickness and/or a width greater than a thickness and/or a width of the respective proximal end sections of the plurality of elongate clot capturing elements of the first retrieval device.

Clause 12. The clot retrieval assembly according to clause 1, wherein the distal end sections of the plurality of elongate clot capturing elements of the second retrieval device have a thickness and/or a width greater than a thickness and/or a width of the respective proximal end sections of the plurality of elongate clot capturing elements of the second retrieval device.

Clause 13. The clot retrieval assembly according to clause 11, wherein the distal end sections of the plurality of elongate clot capturing elements of the second retrieval device has a thickness and/or a width greater than a thickness and/or a width of the respective proximal end sections of the plurality of elongate clot capturing elements of the second retrieval device.

Clause 14. The clot retrieval assembly according to clause 1, wherein the first and second retrieval devices are configured such that upon withdrawing the elongate wire proximally while holding the second tube stationary one of the first and second retrieval devices transitions from the unexpanded state towards or to the expanded state before the other of the first and second retrieval devices transitions from the unexpanded state towards or to the fully expanded state.

Clause 15. The clot retrieval assembly according to clause 14, wherein the second retrieval device is configured to transition from the unexpanded state towards or to the fully expanded state before the first retrieval device transitions from the unexpanded state towards or to the fully expanded state.

Clause 16. The clot retrieval assembly according to clause 1, wherein the proximal end sections of the elongate clot capturing elements of the first retrieval device have a first thickness and the proximal end sections of the elongate clot capturing elements of the second retrieval device have a second thickness that is greater than the first thickness.

Clause 17. The clot retrieval assembly according to clause 1, wherein the proximal end sections of the elongate clot capturing elements of the first retrieval device have a first thickness and the proximal end sections of the elongate clot capturing elements of the second retrieval device have a second thickness that is less than the first thickness.

Clause 18. The clot retrieval assembly according to clause 1, wherein the proximal end sections of the elongate clot capturing elements of the first retrieval device have a first width and the proximal end sections of the elongate clot capturing elements of the second retrieval device have a second width that is greater than the first width.

Clause 19. The clot retrieval assembly according to clause 1, wherein the proximal end sections of the elongate clot capturing elements of the first retrieval device have a first width and the proximal end sections of the elongate clot capturing elements of the second retrieval device have a second width that is less than the first width.

Clause 20. The clot retrieval assembly according to clause 1, further comprising an actuation device having a moveable first part to which a proximal end or proximal end section of the elongate wire is coupled, the actuation device having a stationary second part to which the proximal end or proximal end portion of the second tube is coupled.

Clause 21. The clot retrieval assembly according to clause 1, wherein the proximal end sections of the elongate clot capturing elements of at least one of the first and second retrieval devices are arranged helically with respect to a longitudinal axis of the respective first and second retrieval device.

Clause 22. The clot retrieval assembly according to clause 1, wherein the plurality of elongate clot capturing elements of at least one of the first and second retrieval devices are configured to self-expand to place the at least one of the first and second retrieval devices in a partially expanded state.

Clause 23. The clot retrieval assembly according to clause 1, wherein the plurality of elongate clot capturing elements of both the first and second retrieval devices are configured to self-expand to place both the first and second retrieval devices in a partially expanded state.

Clause 24. The clot retrieval assembly according to clause 23, further comprising a sheath in which the first and second retrieval devices are housed for maintaining each of the first and second clot retrieval devices in the unexpanded state.

Clause 25. The clot retrieval assembly according to clause 1, further comprising a delivery catheter in which the first and second retrieval devices, the first tube, at least a portion of the elongate wire and at least a portion of the second tube are housed, the delivery catheter being configured to deliver the first and second retrieval devices through a vasculature of a patient towards a treatment site.

Clause 26. The clot retrieval assembly according to clause 1, wherein the none of the plurality of elongate clot capturing elements of each of the first and second retrieval devices overlap with one another when the respective first and second retrieval device is in the unexpanded state.

Clause 27. The clot retrieval assembly according to clause 26, wherein at least some of the plurality of elongate clot capturing elements of each of the first and second retrieval devices overlap with one another when the respective first and second retrieval device is in the expanded state.

Clause 28. The clot retrieval assembly according to clause 1, wherein each of the first and second tubes is longitudinally flexible.

Clause 29. The clot retrieval device according to clause 28, wherein at least one of the first and second tubes is made of a metal, the at least one of the first and second tubes including an annular wall comprising a plurality of through holes extending radially through the annular wall, the plurality of through holes being arranged to increase the longitudinal flexibility of the at least one of the first and second tubes compared to the longitudinal flexibility of the at least one of the first and second tubes absent the plurality of through holes.

Clause 30. The clot retrieval assembly according to clause 1, wherein the first retrieval device has a first fully expanded diameter and the second retrieval device has a second fully expanded diameter that is different than the first fully expanded diameter.

Clause 31. The clot retrieval assembly according to clause 1, wherein the first retrieval device has a first fully expanded diameter and the second retrieval device has a second fully expanded diameter that is less than the first fully expanded diameter.

Clause 32. The clot retrieval assembly according to clause 1, wherein each of the elongate clot capturing elements of the first retrieval device have a first thickness and each of the elongate clot capturing elements of the second device has a second thickness different than the first thickness.

Clause 33. The clot retrieval assembly according to clause 1, wherein each of the elongate clot capturing elements of the first retrieval device have a first thickness and each of the elongate clot capturing elements of the second device has a second thickness less than the first thickness.

Clause 34. The clot retrieval assembly according to clause 1, wherein each of the elongate clot capturing elements of the first retrieval device have a first width and each of the elongate clot capturing elements of the second device has a second width different than the first width.

Clause 35. The clot retrieval assembly according to clause 1, wherein each of the elongate clot capturing elements of the first retrieval device have a first width and each of the elongate clot capturing elements of the second device has a second width less than the first width.

### Group D Clauses:

Clause 1. A method for making a clot retrieval assembly, the method comprising:
laser cutting a first tubular member to produce a first retrieval device that includes a proximal collar, a distal collar, and a plurality of elongate clot capturing elements that each has a proximal end attached to the proximal collar and a distal end attached to the distal collar, the first tubular member being laser cut so that each pair of neighboring elongate clot capturing elements is circumferentially separated from one another by an elongate slot that extends continuously between the proximal and distal collars;
laser cutting a second tubular member to produce a second retrieval device that includes a proximal collar, a distal collar, and a plurality of elongate clot capturing elements that each has a proximal end attached to the proximal collar of the second retrieval device and a distal end attached to the distal collar of the second retrieval device, the second tubular member being laser cut so that each pair of neighboring elongate clot capturing elements is circumferentially separated from one another by an elongate slot that extends continuously between the proximal and distal collars of the second retrieval device;
mounting a first tube on an elongate wire, the elongate wire having a proximal end, a distal end, and a longitudinal axis, the first tube having a longitudinal through opening through which the elongate wire passes;
mounting a second tube on the elongate wire at a location proximal to the first tube, the second tube having a longitudinal through opening through which the elongate wire passes;
mounting the first retrieval device on the elongate wire at a location distal to the first tube such that a proximal facing surface of the proximal collar of the first retrieval device faces a distal facing surface of the first tube;
mounting the second retrieval device on the elongate wire at a location between a distal end of the second tube and a proximal end of the first tube such that a proximal facing surface of the proximal collar of the second retrieval device faces a distal facing surface of the second tube and such that a distal facing surface of the distal collar of the second retrieval device faces a proximal facing surface of the first tube; and
operatively coupling a distal end or a distal end portion of the elongate wire to the distal collar of the first retrieval device;
the first and second retrieval devices, elongate wire and first and second tubes being assembled such that as the elongate wire is withdrawn proximally a distance between the proximal and distal collars of each of the first and second retrieval devices decreases to cause a bending of the plurality of elongate clot capturing elements of the respective first and second retrieval devices.

Clause 2. The method according to clause 1, wherein each of the first and second tubular members has an annular wall, the method further comprising reducing a thickness of a portion of the annular wall prior to respectively laser cutting the first and second tubular members.

Clause 3. The method according to clause 2, wherein each of the plurality of elongate capturing elements has a proximal end section and a distal end section. the portion of the annular wall having the reduced thickness residing the proximal end sections of the plurality of elongate clot capturing elements.

Clause 4. The method according to clause 1, wherein each of the plurality of elongate clot capturing elements has a proximal end section and a distal end section, the proximal end sections having a thickness and/or a width less than a thickness and/or a width of the distal end sections.

Clause 5. The method according to clause 1, wherein each of the plurality of elongate clot capturing elements of the first and second retrieval devices includes a proximal end section and a distal end section, the method comprising laser cutting the first and second tubular members so that the proximal end sections of the plurality of elongate clot capturing elements are arranged helically with respect to a longitudinal axis of the respective first and second retrieval devices.

Clause 6. The method according to clause 5, wherein the first tubular member is laser cut so that the distal end sections of the plurality of elongate capture elements are arranged straight and parallel to the longitudinal axis of the first retrieval device, and the second tubular member is laser cut so that the distal end sections of the plurality of elongate capture elements are arranged straight and parallel to the longitudinal axis of the second retrieval device.

Clause 7. The method according to clause 1, wherein after the first tubular member is laser cut to produce the first retrieval device, the plurality of elongate clot capturing elements of the first retrieval device are shape-set while the first retrieval device is in at least a partially expanded state.

Clause 8. The method according to clause 1, wherein after the second tubular member is laser cut to produce the second retrieval device, the plurality of elongate clot capturing elements of the second retrieval device are shape-set while the second retrieval device is in at least a partially expanded state.

Clause 9. The method according to clause 7, wherein after the second tubular member is laser cut to produce the second retrieval device, the plurality of elongate clot capturing elements of the second retrieval device are shape-set while the second retrieval device is in at least a partially expanded state.

Clause 10. The method according to clause 1, wherein after the first tubular member is laser cut to produce the first retrieval device, the plurality of elongate clot capturing elements of the first retrieval device are shape-set while the first retrieval device is in a fully expanded state.

Clause 11. The method according to clause 1, wherein after the second tubular member is laser cut to produce the second retrieval device, the plurality of elongate clot capturing elements of the second retrieval device are shape-set while the second retrieval device is in a fully expanded state.

Clause 12. The method according to clause 10, wherein after the second tubular member is laser cut to produce the second retrieval device, the plurality of elongate clot capturing elements of the second retrieval device are shape-set while the second retrieval device is in a fully expanded state.

Clause 13. The method according to clause 7, wherein the first tubular member is made of a shape memory alloy.

Clause 14. The method according to clause 8, wherein the second tubular member is made of a shape memory alloy.

Clause 15. The method according to clause 9, wherein each of the first and second tubular members is made of a shape memory alloy.

Clause 16. The method according to clause 10, wherein the first tubular member is made of a shape memory alloy.

Clause 17. The method according to clause 11, wherein the second tubular member is made of a shape memory alloy.

Clause 18. The method according to clause 12, wherein each of the first and second tubular members is made of a shape memory alloy.

Clause 19. The method according to clause 15, further comprising constraining the first and second retrieval devices in a sheath to cause each of the first and second retrieval device to assume a radially constrained unexpanded state.

Clause 20. The method according to clause 18, further comprising constraining the first and second retrieval devices in a sheath to cause each of the first and second retrieval device to assume a radially constrained unexpanded state.

### Group E Clauses:

Clause 1. A clot retrieval assembly comprising:
an elongate wire having a proximal end, a distal end, and a longitudinal axis;
a first retrieval device comprising:
   a distal collar operatively coupled with the elongate wire;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
   the first retrieval device being configured to transition from an unexpanded state to an expanded state, in the unexpanded state no portion of the plurality of elongate clot capturing elements is located over the proximal collar, in the expanded state at least some portions of the plurality of elongate clot capturing elements are inverted and residing over the proximal collar;
a second retrieval device comprising:
   a distal collar mounted on the elongate wire, the distal collar having a distal facing surface;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
   the second retrieval device being configured to transition from an unexpanded state to an expanded state, in the unexpanded state no portion of the plurality of elongate clot capturing elements is located over the proximal collar, in the expanded state at least some portions of the plurality of elongate clot capturing elements are inverted and residing over the proximal collar;
a third retrieval device comprising:
   a distal collar mounted on the elongate wire, the distal collar having a distal facing surface;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
   the third retrieval device being configured to transition from an unexpanded state to an expanded state, in the unexpanded state no portion of the plurality of elongate clot capturing elements is located over the proximal collar, in the expanded state at least some portions of the plurality of elongate clot capturing elements are inverted and residing over the proximal collar;
a first tube mounted on the elongate wire and having a longitudinal through opening through which the elongate wire passes, the first tube being located between the proximal collar of the first retrieval device and the distal collar of the second retrieval device, the first tube having a distal facing surface that faces the proximal facing surface of the proximal collar of the first retrieval device, the first tube having a proximal facing surface that faces the distal facing surface of the distal collar of the second retrieval device; and
a second tube mounted on the elongate wire and having a longitudinal through opening through which the elongate wire passes, the second tube being located between the proximal collar of the second retrieval device and the distal collar of the third retrieval device, the second tube having a distal facing surface that faces the proximal facing surface of the proximal collar of the second retrieval device, the second tube having a proximal facing surface that faces the distal facing surface of the distal collar of the third retrieval device;
a third tube mounted on the elongate wire and having a longitudinal through opening through which the elongate wire passes, the proximal collar of the third retrieval device being attached to a distal end of the third tube or otherwise arranged such that the proximal facing surface of the proximal collar of the third retrieval device faces a distal facing surface of the third tube without the proximal collar of the third retrieval device being attached to the third tube;
the first, second and third retrieval devices being configured to transition from their respective unexpanded states to their respective expanded states at least partially by withdrawing the elongate wire proximally while holding the third tube stationary.

Clause 2. The clot retrieval assembly according to clause 1, wherein the first, second and third retrieval devices, elongate wire and first, second and third tubes are assembled such that as the elongate wire is withdrawn proximally a first distance between the proximal and distal collars of the first retrieval device decreases to induce a bending of the plurality of elongate clot capturing elements of the first retrieval device, and a second distance between the proximal and distal collars of the second retrieval device decreases to induce a bending of the plurality of elongate clot capturing elements of the second retrieval device, and a third distance between the proximal and distal collars of the third retrieval device decreases to induce a bending of the plurality of elongate clot capturing elements of the third retrieval device.

Clause 3. The clot retrieval assembly according to clause 1, wherein the distal collar of the first retrieval device is attached to the elongate wire.

Clause 4. The clot retrieval assembly according to clause 1, wherein the distal collar of the first retrieval device is not attached to the elongate wire.

Clause 5. The clot retrieval assembly according to clause 4, further comprising a stop on the distal end or distal end portion of the elongate wire, the stop including a proximal facing surface that faces a distal facing surface of the distal collar of the first retrieval device, the proximal facing surface of the stop being configured to press against the distal facing surface of the distal collar of the first retrieval device upon the elongate wire being withdrawn proximally to cause, or assist in causing, the first retrieval device to transition from the unexpanded state to the expanded state.

Clause 6. The clot retrieval assembly according to clause 5, wherein upon the elongate wire being withdrawn proximally, the proximal facing surface of the proximal collar of the first retrieval device presses against the distal facing surface of the first tube and the proximal facing surface of the first tube presses against the distal facing surface of the distal collar of the second retrieval device to cause, or assist in causing, the second retrieval device to transition from the unexpanded state to the expanded state.

Clause 7. The clot retrieval assembly according to clause 6, wherein upon the elongate wire being withdrawn proximally, the proximal facing surface of the proximal collar of the second retrieval device presses against the distal facing surface of the second tube and the proximal facing surface of the second tube presses against the distal facing surface of the distal collar of the third retrieval device to cause, or assist in causing, the third retrieval device to transition from the unexpanded state to the expanded state.

Clause 8. The clot retrieval assembly according to clause 1, wherein the proximal collar, distal collar and the plurality of elongate clot capturing elements of the first retrieval device are made from a first single piece of material, the proximal collar, distal collar and the plurality of elongate clot capturing elements of the second retrieval device are made from a second single piece of material, and the proximal collar, distal collar and the plurality of elongate clot capturing elements of the third retrieval device are made from a third single piece of material.

Clause 9. The clot retrieval assembly according to clause 8, wherein the plurality of elongate clot capturing elements of the first retrieval device are laser cut from a first metal tubular member, the plurality of elongate clot capturing elements of the second retrieval device are laser cut from a second metal tubular member, and the plurality of elongate clot capturing elements of the third retrieval device are laser cut from a third metal tubular member.

Clause 10. The clot retrieval assembly according to clause 1, wherein the distal end sections of the plurality of elongate clot capturing elements of the first retrieval device have a thickness and/or a width greater than a thickness and/or a width of the respective proximal end sections of the plurality of elongate clot capturing elements of the first retrieval device.

Clause 11. The clot retrieval assembly according to clause 1, wherein the distal end sections of the plurality of elongate clot capturing elements of the second retrieval device has a thickness and/or a width greater than a thickness and/or a width of the respective proximal end sections of the plurality of elongate clot capturing elements of the second retrieval device.

Clause 12. The clot retrieval assembly according to clause 1, wherein the distal end sections of the plurality of elongate clot capturing elements of the third retrieval device has a thickness and/or a width greater than a thickness and/or a width of the respective proximal end sections of the plurality of elongate clot capturing elements of the third retrieval device.

Clause 13. The clot retrieval assembly according to clause 1, wherein the distal end sections of the plurality of elongate clot capturing elements of each of the first, second and third retrieval devices has a thickness and/or a width greater than a thickness and/or a width of the respective proximal end sections of the plurality of elongate clot capturing elements of the first, second and third retrieval device.

Clause 14. The clot retrieval assembly according to clause 1, wherein the first, second and third retrieval devices are configured such that upon withdrawing the elongate wire proximally while holding the third tube stationary, one of the first, second and third retrieval devices transitions from the unexpanded state to the expanded state before the other of the first, second and third retrieval devices transitions from the unexpanded state to the expanded state.

Clause 15. The clot retrieval assembly according to clause 14, wherein the first retrieval device is configured to transition from the unexpanded state to the expanded state before at least one of the second and third retrieval devices transitions from the unexpanded state to the expanded state.

Clause 16. The clot retrieval assembly according to clause 1, wherein the proximal end sections of the elongate clot capturing elements of the first retrieval device have a first thickness and the proximal end sections of the elongate clot capturing elements of the second retrieval device have a second thickness that is greater than the first thickness.

Clause 17. The clot retrieval assembly according to clause 1, wherein the proximal end sections of the elongate clot capturing elements of the first retrieval device have a first thickness and the proximal end sections of the elongate clot capturing elements of the second retrieval device have a second thickness that is less than the first thickness.

Clause 18. The clot retrieval assembly according to clause 1, wherein the proximal end sections of the elongate clot capturing elements of the first retrieval device have a first width and the proximal end sections of the elongate clot capturing elements of the second retrieval device have a second width that is greater than the first width.

Clause 19. The clot retrieval assembly according to clause 1, wherein the proximal end sections of the elongate clot capturing elements of the first retrieval device have a first width and the proximal end sections of the elongate clot capturing elements of the second retrieval device have a second width that is less than the first width.

Clause 20. The clot retrieval assembly according to clause 1, further comprising an actuation device having a moveable first part to which a proximal end or proximal end section of the elongate wire is coupled, the actuation device having a stationary second part to which the proximal end or proximal end portion of the third tube is coupled.

Clause 21. The clot retrieval assembly according to clause 1, wherein the proximal end sections of the elongate clot capturing elements of at least one of the first, second and third retrieval devices are arranged helically with respect to a longitudinal axis of the respective first, second and third retrieval device.

Clause 22. The clot retrieval assembly according to clause 1, wherein the plurality of elongate clot capturing elements of at least one of the first, second and third retrieval devices are configured to self-expand to place the at least one of the first, second and third retrieval devices in a partially expanded state.

Clause 23. The clot retrieval assembly according to clause 1, wherein the plurality of elongate clot capturing elements of each of the first, second and third retrieval devices at are configured to self-expand to place each of the first, second and third retrieval devices in a partially expanded state.

Clause 24. The clot retrieval assembly according to clause 23, further comprising a sheath in which the first, second and third retrieval devices are housed for maintaining each of the first, second and third retrieval devices in the unexpanded state.

Clause 25. The clot retrieval assembly according to clause 1, further comprising a delivery catheter in which the first, second and third retrieval devices, the first and second tubes, at least a portion of the elongate wire and at least a portion of the third tube are housed, the delivery catheter being configured to deliver the first, second and third retrieval devices through a vasculature of a patient towards a treatment site.

Clause 26. The clot retrieval assembly according to clause 1, wherein none of the plurality of elongate clot capturing elements of each of the first, second and third retrieval devices overlap with one another when the respective first, second and third retrieval device is in the unexpanded state.

Clause 27. The clot retrieval assembly according to clause 26, wherein at least some of the plurality of elongate clot capturing elements of each of the first, second and third retrieval devices overlap with one another when the respective first, second and third retrieval device is in the expanded state.

Clause 28. The clot retrieval assembly according to clause 1, wherein each of the first, second and third tubes is flexible.

Clause 29. The clot retrieval device according to clause 28, wherein at least one of the first, second and third tubes is made of a metal, the at least one of the first, second and third tubes including an annular wall comprising a plurality of through holes extending radially through the annular wall, the plurality of through holes being arranged to increase the longitudinal flexibility of the at least one of the first, second and third tubes compared to the longitudinal flexibility of the at least one of the first, second and third tubes absent the plurality of through holes.

Clause 30. The clot retrieval assembly according to clause 1, wherein the first retrieval device has a first fully expanded diameter and the second retrieval device has a second fully expanded diameter that is different than the first fully expanded diameter.

Clause 31. The clot retrieval assembly according to clause 1, wherein the first retrieval device has a first fully expanded diameter and the second retrieval device has a second fully expanded diameter that is less than the first fully expanded diameter.

Clause 32. The clot retrieval assembly according to clause 1, wherein each of the elongate clot capturing elements of the first retrieval device have a first thickness and each of the elongate clot capturing elements of the second device has a second thickness different than the first thickness.

Clause 33. The clot retrieval assembly according to clause 1, wherein each of the elongate clot capturing elements of the first retrieval device have a first thickness and each of the elongate clot capturing elements of the second device has a second thickness less than the first thickness.

Clause 34. The clot retrieval device according to clause 1, wherein the first tube has a first length and the second tube has a second length different than the first length.

Clause 35. The clot retrieval assembly according to clause 1, wherein the plurality of elongate clot capturing elements of the first, second and third retrieval device are laser cut from a same tubular member.

Clause 36. The clot retrieval assembly according to clause 1, wherein the first retrieval device, second retrieval device, third retrieval device, first tube and second tube are made from a single piece of material.

### Group F Clauses:

Clause 1. A method for making a clot retrieval assembly, the method comprising:
laser cutting a first tubular member to produce a first retrieval device that includes a proximal collar, a distal collar, and a plurality of elongate clot capturing elements that each has a proximal end attached to the proximal collar and a distal end attached to the distal collar, the first tubular member being laser cut so that each pair of neighboring elongate clot capturing elements is circumferentially separated from one another by an elongate slot that extends continuously between the proximal and distal collars;
laser cutting a second tubular member to produce a second retrieval device that includes a proximal collar, a distal collar, and a plurality of elongate clot capturing elements that each has a proximal end attached to the proximal collar and a distal end attached to the distal collar, the second tubular member being laser cut so that each pair of neighboring elongate clot capturing elements is circumferentially separated from one another by an elongate slot that extends continuously between the proximal and distal collars;
laser cutting a third tubular member to produce a third retrieval device that includes a proximal collar, a distal collar, and a plurality of elongate clot capturing elements that each has a proximal end attached to the proximal collar and a distal end attached to the distal collar, the third tubular member being laser cut so that each pair of neighboring elongate clot capturing elements is circumferentially separated from one another by an elongate slot that extends continuously between the proximal and distal collars;
mounting a first tube on an elongate wire, the elongate wire having a proximal end, a distal end, and a longitudinal axis, the first tube having a longitudinal through opening through which the elongate wire passes;
mounting a second tube on the elongate wire at a location proximal to the first tube, the second tube having a longitudinal through opening through which the elongate wire passes;
mounting a third tube on the elongate wire at a location proximal to the second tube, the third tube having a longitudinal through opening through which the elongate wire passes;
mounting the first retrieval device on the elongate wire at a location distal to the first tube, the proximal collar of the first retrieval device having a proximal facing surface that faces a distal facing surface of the first tube;
mounting the second retrieval device on the elongate wire at a location between a distal end of the second tube and a proximal end of the first tube such that a proximal facing surface of the proximal collar of the second retrieval device faces a distal facing surface of the second tube and such that a distal facing surface of the distal collar of the second retrieval device faces a proximal facing surface of the first tube;
mounting the third retrieval device on the elongate wire at a location between a distal end of the third tube and a proximal end of the second tube such that the proximal collar of the third retrieval device is coupled to a distal end of the third tube or such that a proximal facing surface of the proximal collar of the third retrieval device faces a distal facing surface of the third tube, and such that a distal facing surface of the distal collar of the third retrieval device faces a proximal facing surface of the second tube;
operatively coupling a distal end or a distal end portion of the elongate wire to the distal collar of the first retrieval device;
the first, second and third retrieval devices, elongate wire and first, second and third tubes being assembled such that as the elongate wire is withdrawn proximally a distance between the proximal and distal collars of each of the first, second and third retrieval devices decreases to cause a bending of the plurality of elongate clot capturing elements of the respective first, second and third retrieval devices.

Clause 2. The method according to clause 1, wherein each of the first, second and third tubular members has an annular wall, the method further comprising reducing a thickness of a portion of the annular wall prior to respectively laser cutting the first, second and third tubular members.

Clause 3. The method according to clause 2, wherein each of the plurality of elongate capturing elements has a proximal end section and a distal end section. the portion of the annular wall corresponding to the proximal end sections of the plurality of elongate clot capturing elements.

Clause 4. The method according to clause 1, wherein each of the plurality of elongate clot capturing elements has a proximal end section and a distal end section, the proximal end sections having a thickness and/or a width less than a thickness and/or a width of the distal end sections.

Clause 5. The method according to clause 1, wherein each of the plurality of elongate clot capturing elements of the first and second retrieval devices includes a proximal end section and a distal end section, the method comprising laser cutting the first and second tubular members so that the proximal end sections of the plurality of elongate clot capturing elements are arranged helically with respect to a longitudinal axis of the respective first and second retrieval devices.

Clause 6. The method according to clause 5, wherein the first tubular member is laser cut so that the distal end sections of the plurality of elongate capture elements are arranged straight and parallel to the longitudinal axis of the first retrieval device, and the second tubular member is laser cut so that the distal end sections of the plurality of elongate capture elements are arranged straight and parallel to the longitudinal axis of the second retrieval device.

Clause 7. The method according to clause 1, wherein after the first tubular member is laser cut to produce the first retrieval device, the plurality of elongate clot capturing elements of the first retrieval device are shape-set while the first retrieval device is in at least a partially expanded state.

Clause 8. The method according to clause 1, wherein after the second tubular member is laser cut to produce the second retrieval device, the plurality of elongate clot capturing elements of the second retrieval device are shape-set while the second retrieval device is in at least a partially expanded state.

Clause 9. The method according to clause 7, wherein after the second tubular member is laser cut to produce the second retrieval device, the plurality of elongate clot capturing elements of the second retrieval device are shape-set while the second retrieval device is in at least a partially expanded state.

Clause 10. The method according to clause 1, wherein after the first tubular member is laser cut to produce the first retrieval device, the plurality of elongate clot capturing elements of the first retrieval device are shape-set while the first retrieval device is in a fully expanded state.

Clause 11. The method according to clause 1, wherein after the second tubular member is laser cut to produce the second retrieval device, the plurality of elongate clot capturing elements of the second retrieval device are shape-set while the second retrieval device is in a fully expanded state.

Clause 12. The method according to clause 10, wherein after the second tubular member is laser cut to produce the second retrieval device, the plurality of elongate clot capturing elements of the second retrieval device are shape-set while the second retrieval device is in a fully expanded state.

Clause 13. The method according to clause 7, wherein the first tubular member is made of a shape memory alloy.

Clause 14. The method according to clause 8, wherein the second tubular member is made of a shape memory alloy.

Clause 15. The method according to clause 9, wherein each of the first and second tubular members is made of a shape memory alloy.

Clause 16. The method according to clause 10, wherein the first tubular member is made of a shape memory alloy.

Clause 17. The method according to clause 11, wherein the second tubular member is made of a shape memory alloy.

Clause 18. The method according to clause 12, wherein each of the first and second tubular members is made of a shape memory alloy.

Clause 19. The method according to clause 15, further comprising constraining the first and second retrieval devices in a sheath to cause each of the first and second retrieval device to assume a radially constrained unexpanded state.

Clause 20. The method according to clause 18, further comprising constraining the first and second retrieval devices in a sheath to cause each of the first and second retrieval device to assume a radially constrained unexpanded state.

### Group G Clauses:

Clause 1. An assembly for retrieving a clot from a bodily duct of a patient, the assembly comprising:
an elongate wire having a length, a longitudinal axis, a proximal end portion intended to reside outside the bodily duct of the patient during a retrieving of the clot from the bodily duct, and a distal end portion intended to reside inside the bodily duct of the patient during a retrieving of the clot from the bodily duct;
a retrieval device mounted on the elongate wire, the retrieval device having a central longitudinal axis and being capable of transitioning from a radially unexpanded state to a radially expanded state, in the radially unexpanded state the retrieval device is configured to be delivered to inside the bodily duct of the patient to a location distal to the clot, in the radially expanded state the retrieval device is configured to retrieve the clot, the retrieval device comprising:
   a distal collar having a first opening through which a first portion of the distal end portion of the elongate wire resides;
   a proximal collar located proximal to the distal collar, the proximal collar having a second opening through which a second portion of the distal end portion of the elongate wire passes, when the retrieval device is in the unexpanded state the first and second collars are separated by a first distance, and when the retrieval device is in the expanded state the first and second collars are separated by a second distance that is less than the first distance, at least one of the proximal collar and distal collar being slideable along a portion of the length of the elongate wire to enable the retrieval device to transition from the unexpanded state to the expanded state;
   a plurality of elongate clot capturing elements in which each pair of neighboring elongate clot capturing elements is circumferentially separated from one another by an elongate slot that extends continuously between the proximal and distal collars, each of the plurality of elongated clot capturing elements has a proximal end attached to the proximal collar and a distal end attached to the distal collar, the elongate clot capturing elements having a proximal end section, a distal end section and a junction where the proximal and distal end sections meet, the proximal end of one or more of the elongate clot capturing elements having a first width, the distal end of the one or more elongate clot capturing elements having a second width and the junction having a third width, a portion of the proximal end section located between the proximal end of the one or more elongate clot capturing elements and the junction having a fourth width and a portion of the distal end section located between the distal end of the one or more elongate clot capturing elements and the junction having a fifth width, the third width being greater than each of the fourth and fifth widths, the one or more elongate clot capturing elements being tapered between the portion of the proximal end section and the junction and between the portion of the proximal end section and the junction.

Clause 2. The assembly according to clause 1, wherein the one or more elongate clot capturing elements are tapered between the proximal end and the portion of the proximal end section and are tapered between the distal end and the portion of the distal end section.

Clause 3. The assembly according to clause 1, wherein the first width, second width and third width are substantially the same.

Clause 4. The assembly according to clause 1, wherein the first width, second width and third width are substantially the same and the fourth width and fifth width are substantially the same.

Clause 5. The assembly according to clause 1, wherein the first width and second width are not substantially the same as the third width.

Clause 6. The assembly according to clause 5, wherein the first width and second width are substantially the same.

Clause 6. The assembly according to clause 1, wherein the fourth width and fifth width are not substantially the same.

Clause 7. The assembly according to clause 4, wherein w1, w2 and w3 are maximum widths along the length of the one or more elongate clot capturing elements and w4 and w5 are minimum widths along the length of the elongate clot capturing elements.

Clause 8. The assembly according to clause to clause 7, wherein w4 is a width located at a midpoint between the proximal end and junction of the one or more clot capturing elements, and w5 is a width located at a midpoint between the distal end and junction of the one or more clot capturing elements.

Clause 9. The assembly according to clause 2, wherein the one or more elongate clot capturing elements have a first side and a second side that are both tapered.

Clause 10. The assembly according to clause 1, wherein the ratio of w3/w2 and the ratio of w3/w4 is 1.3 to 1.9.

Clause 11. The assembly according to clause 1, wherein at least a part of one or both of the proximal and distal end sections of the elongate clot capturing elements is arranged curved about the longitudinal axis of the retrieval device when the retrieval device is in the unexpanded state.

Clause 12. The assembly according to clause 1, wherein at least a part of the one or both of the proximal and distal end sections of the elongate clot capturing elements being arranged in a helical pattern about the longitudinal axis of the retrieval device when the retrieval device is in the unexpanded state.

Clause 13. The assembly according to clause 1, wherein at least a majority of the length of one or both the proximal and distal end sections of the elongate clot capturing elements being arranged in a helical pattern about the longitudinal axis of the retrieval device when the retrieval device is in the unexpanded state.

Clause 14. The assembly according to clause 1, wherein at least a part of the proximal end section of the elongate clot capturing elements is arranged in a helical pattern about the longitudinal axis of the retrieval device when the retrieval device is in the unexpanded state, and the distal end section of the elongate clot capturing elements being arranged parallel to the longitudinal axis of the retrieval device when the retrieval device is in the unexpanded state.

Clause 15. The assembly according to clause 1, wherein at least a part of the distal end section of the elongate clot capturing elements is arranged in a helical pattern about the longitudinal axis of the retrieval device when the retrieval device is in the unexpanded state, and the proximal end section of the elongate clot capturing elements being arranged parallel to the longitudinal axis of the retrieval device when the retrieval device is in the unexpanded state.

Clause 16. The assembly according to clause 1, wherein at least a majority of the length of the proximal end section of the elongate clot capturing elements is arranged in a helical pattern about the longitudinal axis of the retrieval device when the retrieval device is in the unexpanded state, and at least a majority of the length of the distal end section of the elongate clot capturing elements being arranged parallel to the longitudinal axis of the retrieval device when the retrieval device is in the unexpanded state.

Clause 17. The assembly according to clause 1, wherein at least a majority of the length of the distal end section of the elongate clot capturing elements is arranged in a helical pattern about the longitudinal axis of the retrieval device when the retrieval device is in the unexpanded state, and at least a majority of the length of the proximal end section of the elongate clot capturing elements being arranged parallel to the longitudinal axis of the retrieval device when the retrieval device is in the unexpanded state.

Clause 18. The assembly according to clause 1, wherein the at least a part of the proximal end section of the elongate clot capturing elements being arranged in a first helical pattern about the longitudinal axis of the retrieval device when the retrieval device is in the unexpanded state, and at least a part of the distal end section of the elongate clot capturing elements being arranged in a second helical pattern about the longitudinal axis of the retrieval device when the retrieval device is in the unexpanded state, the second helical pattern being different than the first helical pattern.

Clause 19. The assembly according to clause 1, wherein the at least a majority of the length of the proximal end section of the elongate clot capturing elements being arranged in a first helical pattern about the longitudinal axis of the retrieval device when the retrieval device is in the unexpanded state, and at least a majority of the length of the distal end section of the elongate clot capturing elements being arranged in a second helical pattern about the longitudinal axis of the retrieval device when the retrieval device is in the unexpanded state, the second helical pattern being different than the first helical pattern.

Clause 20. The assembly according to clause 19, wherein the first helical pattern corresponds to the proximal end section of the elongate clot capturing elements curving in a first direction and the second helical pattern corresponds to the distal end section of the elongate clot capturing elements curving in a second direction opposite the first direction.

Clause 21. The assembly according to clause 1, wherein one or both of the proximal and distal collars rotates as the retrieval device transitions from the unexpanded state to the expanded state.

Clause 22. The assembly according to clause 1, wherein when the retrieval device is in the unexpanded state no portion of each of the plurality of elongate clot capturing elements bends back on itself.

Clause 23. The assembly according to clause 1, wherein when the retrieval device is in the unexpanded state no portion of the plurality of elongate clot capturing elements is located proximal to the proximal collar.

Clause 24. The assembly according to clause 1, wherein when the retrieval device is in the expanded state at least portions of some or all of the plurality of elongate clot capturing elements extend proximal to the proximal collar.

Clause 25. The assembly according to clause 1, wherein the plurality of elongate clot capturing elements and the proximal and distal collars are formed from a single piece of material.

Clause 26. The assembly according to clause 1, wherein the distal collar is fixed stationary on the elongate wire.

Clause 27. The assembly according to clause 1, wherein the distal end sections of the elongate clot capturing elements overlap with one another when the retrieval device is in the expanded state and do not overlap with one another when the retrieval device is in the unexpanded state.

Clause 28. The assembly according to clause 25, wherein the plurality of elongate clot capturing elements are laser cut from a tubular member.

### Group H Clauses:

Clause 1. An obstruction retrieval device comprising:
a retrieval device having a longitudinal axis and capable of transitioning from a radially unexpanded state to a radially expanded state, in the radially unexpanded state the retrieval device is configured to be delivered to inside a bodily duct of a patient to a location distal to the obstruction, in the radially expanded state the retrieval device is configured to retrieve the obstruction, the retrieval device comprising:
   a distal collar having a first longitudinal through opening;
   a proximal collar located proximal to the distal collar, the proximal collar having a second longitudinal through opening, when the retrieval device is in the unexpanded state the first and second collars are separated by a first distance, and when the retrieval device is in the expanded state the first and second collars are separated by a second distance that is less than the first distance; and
   a plurality of elongate clot capturing elements in which each pair of neighboring elongate clot capturing elements is circumferentially separated from one another by an elongate slot that extends continuously between the proximal and distal collars, each of the plurality of elongated clot capturing elements has a proximal end attached to the proximal collar and a distal end attached to the distal collar, the elongate clot capturing elements having a proximal end section, a distal end section and a junction where the proximal and distal end sections meet, the proximal end of one or more of the elongate clot capturing elements having a first width, the distal end of the one or more elongate clot capturing elements having a second width and the junction having a third width, a portion of the proximal end section located between the proximal end of the one or more elongate clot capturing elements and the junction having a fourth width and a portion of the distal end section located between the distal end of the one or more elongate clot capturing elements and the junction having a fifth width, the third width being greater than each of the fourth and fifth widths, the one or more elongate clot capturing elements being tapered between the portion of the proximal end section and the junction and between the portion of the proximal end section and the junction.

Clause 2. The obstruction retrieval device according to clause 1, wherein the one or more elongate clot capturing elements are tapered between the proximal end and the portion of the proximal end section and are tapered between the distal end and the portion of the distal end section.

Clause 3. The obstruction retrieval device according to clause 1, wherein the first width, second width and third width are substantially the same.

Clause 4. The obstruction retrieval device according to clause 1, wherein the first width, second width and third width are substantially the same and the fourth width and fifth width are substantially the same.

Clause 5. The obstruction retrieval device according to clause 1, wherein the first width and second width are not substantially the same as the third width.

Clause 6. The obstruction retrieval device according to clause 5, wherein the first width and second width are substantially the same.

Clause 7. The obstruction retrieval device according to clause 1, wherein the fourth width and fifth width are not substantially the same.

Clause 8. The obstruction retrieval device according to clause 4, wherein w1, w2 and w3 are maximum widths along the length of the one or more elongate clot capturing elements and w4 and w5 are minimum widths along the length of the elongate clot capturing elements.

Clause 9. The obstruction retrieval device according to clause to clause 8, wherein w4 is a width located at a midpoint between the proximal end and junction of the one or more clot capturing elements, and w5 is a width located at a midpoint between the distal end and junction of the one or more clot capturing elements.

Clause 10. The obstruction retrieval device according to clause 2, wherein the one or more elongate clot capturing elements have a first side and a second side that are both tapered.

Clause 11. The obstruction retrieval device according to clause 1, wherein the ratio of w3/w2 and the ratio of w3/w4 is 1.3 to 1.9.

Clause 12. The obstruction retrieval device according to clause 1, wherein at least a part of one or both of the proximal and distal end sections of the elongate clot capturing elements is arranged curved about the longitudinal axis of the retrieval device when the retrieval device is in the unexpanded state.

Clause 13. The obstruction retrieval device according to clause 1, wherein at least a part of the one or both of the proximal and distal end sections of the elongate clot capturing elements being arranged in a helical pattern about the longitudinal axis of the retrieval device when the retrieval device is in the unexpanded state.

Clause 14. The obstruction retrieval device according to clause 1, wherein at least a majority of the length of one or both the proximal and distal end sections of the elongate clot capturing elements being arranged in a helical pattern about the longitudinal axis of the retrieval device when the retrieval device is in the unexpanded state.

Clause 15. The obstruction retrieval device according to clause 1, wherein at least a part of the proximal end section of the elongate clot capturing elements is arranged in a helical pattern about the longitudinal axis of the retrieval device when the retrieval device is in the unexpanded state, and the distal end section of the elongate clot capturing elements being arranged parallel to the longitudinal axis of the retrieval device when the retrieval device is in the unexpanded state.

Clause 16. The obstruction retrieval device according to clause 1, wherein at least a part of the distal end section of the elongate clot capturing elements is arranged in a helical pattern about the longitudinal axis of the retrieval device when the retrieval device is in the unexpanded state, and the proximal end section of the elongate clot capturing elements being arranged parallel to the longitudinal axis of the retrieval device when the retrieval device is in the unexpanded state.

Clause 17. The obstruction retrieval device according to clause 1, wherein at least a majority of the length of the proximal end section of the elongate clot capturing elements is arranged in a helical pattern about the longitudinal axis of the retrieval device when the retrieval device is in the unexpanded state, and at least a majority of the length of the distal end section of the elongate clot capturing elements being arranged parallel to the longitudinal axis of the retrieval device when the retrieval device is in the unexpanded state.

Clause 18. The obstruction retrieval device according to clause 1, wherein at least a majority of the length of the distal end section of the elongate clot capturing elements is arranged in a helical pattern about the longitudinal axis of the retrieval device when the retrieval device is in the unexpanded state, and at least a majority of the length of the proximal end section of the elongate clot capturing elements being arranged parallel to the longitudinal axis of the retrieval device when the retrieval device is in the unexpanded state.

Clause 19. The obstruction retrieval device according to clause 1, wherein the at least a part of the proximal end section of the elongate clot capturing elements being arranged in a first helical pattern about the longitudinal axis of the retrieval device when the retrieval device is in the unexpanded state, and at least a part of the distal end section of the elongate clot capturing elements being arranged in a second helical pattern about the longitudinal axis of the retrieval device when the retrieval device is in the unexpanded state, the second helical pattern being different than the first helical pattern.

Clause 20. The obstruction retrieval device according to clause 1, wherein the at least a majority of the length of the proximal end section of the elongate clot capturing elements being arranged in a first helical pattern about the longitudinal axis of the retrieval device when the retrieval device is in the unexpanded state, and at least a majority of the length of the distal end section of the elongate clot capturing elements being arranged in a second helical pattern about the longitudinal axis of the retrieval device when the retrieval device is in the unexpanded state, the second helical pattern being different than the first helical pattern.

Clause 21. The obstruction retrieval device according to clause 20, wherein the first helical pattern corresponds to the proximal end section of the elongate clot capturing elements curving in a first direction and the second helical pattern corresponds to the distal end section of the elongate clot capturing elements curving in a second direction opposite the first direction.

Clause 22. The obstruction retrieval device according to clause 1, wherein one or both of the proximal and distal collars rotates as the retrieval device transitions from the unexpanded state to the expanded state.

Clause 23. The obstruction retrieval device according to clause 1, wherein when the retrieval device is in the unexpanded state no portion of each of the plurality of elongate clot capturing elements bends back on itself.

Clause 24. The obstruction retrieval device according to clause 1, wherein when the retrieval device is in the unexpanded state no portion of the plurality of elongate clot capturing elements is located proximal to the proximal collar.

Clause 25. The obstruction retrieval device according to clause 1, wherein when the retrieval device is in the expanded state at least portions of some or all of the plurality of elongate clot capturing elements extend proximal to the proximal collar.

Clause 26. The obstruction retrieval device according to clause 1, wherein the plurality of elongate clot capturing elements and the proximal and distal collars are formed from a single piece of material.

Clause 27. The obstruction retrieval device according to clause 1, wherein the distal collar is fixed stationary on the elongate wire.

Clause 28. The obstruction retrieval device according to clause 1, wherein the distal end sections of the elongate clot capturing elements overlap with one another when the retrieval device is in the expanded state and do not overlap with one another when the retrieval device is in the unexpanded state.

Clause 29. The obstruction retrieval device according to clause 26, wherein the plurality of elongate clot capturing elements are laser cut from a tubular member.

### Group I Clauses:

Clause 1. A clot retrieval assembly comprising:
an elongate wire having a proximal end, a distal end, and a longitudinal axis;
a retrieval device comprising:
   a distal collar operatively coupled with the elongate wire;
   a proximal collar mounted on the elongate wire and having a longitudinal through opening through which the elongate wire passes, the proximal collar having a proximal facing surface;
a plurality of elongate clot capturing elements that each has a proximal end attached to the proximal collar and a distal end attached to the distal collar, each of the plurality of elongate capturing elements having a proximal end section and a distal end section; the retrieval device being configured to transition from an unexpanded state to a fully expanded state, in the unexpanded state no portion of the plurality of elongate clot capturing elements is located over the proximal collar, in the fully expanded state at least portions of the plurality of elongate clot capturing elements are inverted and residing over the proximal collar; and
a tube mounted on the elongate wire and having a longitudinal through opening through which the elongate wire passes, the proximal collar being attached to a distal end of the tube or otherwise arranged such that the proximal facing surface of the proximal collar faces a distal facing surface of the tube without the proximal collar being attached to the tube;
the retrieval device being configured to transition towards or to a fully expanded state at least partially by advancing the tube distally.

Clause 2: The clot retrieval assembly according to clause 1, wherein the retrieval device is configured to transition towards or to a fully expanded state at least partially by advancing the tube distally while holding the elongate wire stationary.

Clause 3. A clot retrieval assembly comprising:
an elongate wire having a proximal end, a distal end, and a longitudinal axis;
a first retrieval device mounted on the elongate wire and comprising:
   a distal collar operatively coupled with the elongate wire;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
   the first retrieval device being configured to transition from an unexpanded state to a fully expanded state, in the unexpanded state no portion of the plurality of elongate clot capturing elements is located over the proximal collar, in the fully expanded state at least some of portions of the plurality of elongate clot capturing elements are inverted and residing over the proximal collar;
a second retrieval device mounted on the elongate wire at a location proximal to the first retrieval device, the second retrieval device comprising:
   a distal collar mounted on the elongate wire, the distal collar of the second retrieval device having a distal facing surface;
   a proximal collar mounted on the elongate wire and having a longitudinal through opening through which the elongate wire passes, the proximal collar of the second retrieval device having a proximal facing surface;
   a plurality of elongate clot capturing elements that each has a proximal end attached to the proximal collar of the second retrieval device and a distal end attached to the distal collar of the second retrieval device, each of the plurality of elongate capturing elements of the second retrieval device having a proximal end portion and a distal end portion, each pair of neighboring elongate clot capturing elements of the second retrieval device being circumferentially separated from one another by an elongate slot that extends continuously between the proximal and distal collars of the second retrieval devices;
   the second retrieval device being configured to transition from an unexpanded state to the fully expanded state, in the unexpanded state no portion of the plurality of elongate clot capturing elements of the second retrieval device is located over the proximal collar, in the fully expanded state at least some portions of the plurality of elongate clot capturing elements of the second retrieval device are inverted and residing over the proximal collar;
a first tube mounted on the elongate wire between the proximal collar of the first retrieval device and the distal collar of the second retrieval device, the first tube having a distal facing surface that faces the proximal facing surface of the proximal collar of the first retrieval device, the first tube having a proximal facing surface that faces the distal facing surface of the distal collar of the second retrieval device; and
a second tube mounted on the elongate wire and having a longitudinal through opening through which the elongate wire passes, the proximal collar of the second retrieval device being attached to a distal end of the second tube or otherwise arranged such that the proximal facing surface of the proximal collar of the second retrieval device faces a distal facing surface of the second tube without the proximal collar being attached to the second tube;
the first and second retrieval devices being configured to transition from their respective unexpanded states towards or to their respective fully expanded states at least partially by advancing the second tube distally.

Clause 4. The clot retrieval assembly according to clause 3, wherein the first and second retrieval devices being configured to transition from their respective unexpanded states towards or to their respective fully expanded states at least partially by advancing the second tube distally while holding the elongate wire stationary.

Clause 5. A clot retrieval assembly comprising:
an elongate wire having a proximal end, a distal end, and a longitudinal axis;
a first retrieval device comprising:
   a distal collar operatively coupled with the elongate wire;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
   the first retrieval device being configured to transition from an unexpanded state to an expanded state, in the unexpanded state no portion of the plurality of elongate clot capturing elements is located over the proximal collar, in the expanded state at least some portions of the plurality of elongate clot capturing elements are inverted and residing over the proximal collar;
a second retrieval device comprising:
   a distal collar mounted on the elongate wire, the distal collar having a distal facing surface;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
   the second retrieval device being configured to transition from an unexpanded state to an expanded state, in the unexpanded state no portion of the plurality of elongate clot capturing elements is located over the proximal collar, in the expanded state at least some portions of the plurality of elongate clot capturing elements are inverted and residing over the proximal collar;
a third retrieval device comprising:
   a distal collar mounted on the elongate wire, the distal collar having a distal facing surface;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
   the third retrieval device being configured to transition from an unexpanded state to an expanded state, in the unexpanded state no portion of the plurality of elongate clot capturing elements is located over the proximal collar, in the expanded state at least some portions of the plurality of elongate clot capturing elements are inverted and residing over the proximal collar;
a first tube mounted on the elongate wire and having a longitudinal through opening through which the elongate wire passes, the first tube being located between the proximal collar of the first retrieval device and the distal collar of the second retrieval device, the first tube having a distal facing surface that faces the proximal facing surface of the proximal collar of the first retrieval device, the first tube having a proximal facing surface that faces the distal facing surface of the distal collar of the second retrieval device; and
a second tube mounted on the elongate wire and having a longitudinal through opening through which the elongate wire passes, the second tube being located between the proximal collar of the second retrieval device and the distal collar of the third retrieval device, the second tube having a distal facing surface that faces the proximal facing surface of the proximal collar of the second retrieval device, the second tube having a proximal facing surface that faces the distal facing surface of the distal collar of the third retrieval device;
a third tube mounted on the elongate wire and having a longitudinal through opening through which the elongate wire passes, the proximal collar of the third retrieval device being attached to a distal end of the third tube or otherwise arranged such that the proximal facing surface of the proximal collar of the third retrieval device faces a distal facing surface of the third tube without the proximal collar of the third retrieval device being attached to the third tube;
the first, second and third retrieval devices being configured to transition from their respective unexpanded states to their respective expanded states at least partially by advancing the third tube distally.

Clause 6. The clot retrieval assembly according to clause 5, wherein the first, second and third retrieval devices being configured to transition from their respective unexpanded states to their respective expanded states at least partially by advancing the third tube distally while holding the elongate wire stationary.

Clause 7. A clot retrieval assembly comprising:
an elongate wire having a proximal end, a distal end, and a longitudinal axis;
a retrieval device comprising:
   a distal collar operatively coupled with the elongate wire;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section;
   the retrieval device being configured to transition from an unexpanded state to a fully expanded state, in the unexpanded state no portion of the plurality of elongate clot capturing elements is located over the proximal collar, in the fully expanded state at least portions of the plurality of elongate clot capturing elements are inverted and residing over the proximal collar; and
a tube mounted on the elongate wire and having a longitudinal through opening through which the elongate wire passes, the proximal collar being attached to a distal end of the tube or otherwise arranged such that the proximal facing surface of the proximal collar faces a distal facing surface of the tube without the proximal collar being attached to the tube;
the retrieval device being configured to transition towards or to a fully expanded state at least partially by withdrawing the elongate wire proximally and advancing the tube distally.

Clause 8. A clot retrieval assembly comprising:
an elongate wire having a proximal end, a distal end, and a longitudinal axis;
a first retrieval device mounted on the elongate wire and comprising:
   a distal collar operatively coupled with the elongate wire;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
   the first retrieval device being configured to transition from an unexpanded state to a fully expanded state, in the unexpanded state no portion of the plurality of elongate clot capturing elements is located over the proximal collar, in the fully expanded state at least some of portions of the plurality of elongate clot capturing elements are inverted and residing over the proximal collar;
a second retrieval device mounted on the elongate wire at a location proximal to the first retrieval device, the second retrieval device comprising:
   a distal collar mounted on the elongate wire, the distal collar of the second retrieval device having a distal facing surface;
   a proximal collar mounted on the elongate wire and having a longitudinal through opening through which the elongate wire passes, the proximal collar of the second retrieval device having a proximal facing surface;
   a plurality of elongate clot capturing elements that each has a proximal end attached to the proximal collar of the second retrieval device and a distal end attached to the distal collar of the second retrieval device, each of the plurality of elongate capturing elements of the second retrieval device having a proximal end portion and a distal end portion, each pair of neighboring elongate clot capturing elements of the second retrieval device being circumferentially separated from one another by an elongate slot that extends continuously between the proximal and distal collars of the second retrieval devices;
   the second retrieval device being configured to transition from an unexpanded state to the fully expanded state, in the unexpanded state no portion of the plurality of elongate clot capturing elements of the second retrieval device is located over the proximal collar, in the fully expanded state at least some portions of the plurality of elongate clot capturing elements of the second retrieval device are inverted and residing over the proximal collar;
a first tube mounted on the elongate wire between the proximal collar of the first retrieval device and the distal collar of the second retrieval device, the first tube having a distal facing surface that faces the proximal facing surface of the proximal collar of the first retrieval device, the first tube having a proximal facing surface that faces the distal facing surface of the distal collar of the second retrieval device; and
a second tube mounted on the elongate wire and having a longitudinal through opening through which the elongate wire passes, the proximal collar of the second retrieval device being attached to a distal end of the second tube or otherwise arranged such that the proximal facing surface of the proximal collar of the second retrieval device faces a distal facing surface of the second tube without the proximal collar being attached to the second tube;
the first and second retrieval devices being configured to transition from their respective unexpanded states towards or to their respective fully expanded states at least partially by withdrawing the elongate wire proximally and advancing the second tube distally.

Clause 9. A clot retrieval assembly comprising:
an elongate wire having a proximal end, a distal end, and a longitudinal axis;
a first retrieval device comprising:
   a distal collar operatively coupled with the elongate wire;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
   the first retrieval device being configured to transition from an unexpanded state to an expanded state, in the unexpanded state no portion of the plurality of elongate clot capturing elements is located over the proximal collar, in the expanded state at least some portions of the plurality of elongate clot capturing elements are inverted and residing over the proximal collar;
a second retrieval device comprising:
   a distal collar mounted on the elongate wire, the distal collar having a distal facing surface;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
   the second retrieval device being configured to transition from an unexpanded state to an expanded state, in the unexpanded state no portion of the plurality of elongate clot capturing elements is located over the proximal collar, in the expanded state at least some portions of the plurality of elongate clot capturing elements are inverted and residing over the proximal collar;
a third retrieval device comprising:
   a distal collar mounted on the elongate wire, the distal collar having a distal facing surface;
   a proximal collar mounted on the elongate wire and having a longitudinal through opening through which the elongate wire passes, the proximal collar having a proximal facing surface;
   a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
   the third retrieval device being configured to transition from an unexpanded state to an expanded state, in the unexpanded state no portion of the plurality of elongate clot capturing elements is located over the proximal collar, in the expanded state at least some portions of the plurality of elongate clot capturing elements are inverted and residing over the proximal collar;
a first tube mounted on the elongate wire and having a longitudinal through opening through which the elongate wire passes, the first tube being located between the proximal collar of the first retrieval device and the distal collar of the second retrieval device, the first tube having a distal facing surface that faces the proximal facing surface of the proximal collar of the first retrieval device, the first tube having a proximal facing surface that faces the distal facing surface of the distal collar of the second retrieval device; and
a second tube mounted on the elongate wire and having a longitudinal through opening through which the elongate wire passes, the second tube being located between the proximal collar of the second retrieval device and the distal collar of the third retrieval device, the second tube having a distal facing surface that faces the proximal facing surface of the proximal collar of the second retrieval device, the second tube having a proximal facing surface that faces the distal facing surface of the distal collar of the third retrieval device;
a third tube mounted on the elongate wire and having a longitudinal through opening through which the elongate wire passes, the proximal collar of the third retrieval device being attached to a distal end of the third tube or otherwise arranged such that the proximal facing surface of the proximal collar of the third retrieval device faces a distal facing surface of the third tube without the proximal collar of the third retrieval device being attached to the third tube; the first, second and third retrieval devices being configured to transition from their respective unexpanded states to their respective expanded states at least partially by withdrawing the elongate wire proximally and advancing the third tube distally.

What is claimed is:

1. A clot retrieval assembly comprising:
an elongate wire having a proximal end, a distal end, and a longitudinal axis;
a first retrieval device comprising:
a distal collar operatively coupled with the elongate wire;
a proximal collar mounted on the elongate wire and having a longitudinal through opening through which the elongate wire passes, the proximal collar having a proximal facing surface;
a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements is circumferentially separated from one another by an elongate slot that extends continuously between the proximal and distal collars;
a first tube mounted on the elongate wire and having a longitudinal through opening through which the elongate wire passes, the first tube having a distal facing surface that faces the proximal facing surface of the proximal collar of the first retrieval device;
the first retrieval device being configured to transition from an unexpanded state to a fully expanded state, in the unexpanded state no portion of the plurality of elongate clot capturing elements is located over the proximal collar, in the fully expanded state at least some of the proximal portions of the plurality of elongate clot capturing elements are inverted and residing over the proximal collar; and
the first retrieval device being configured to transition towards or to a fully expanded state at least partially by withdrawing the elongate wire proximally to cause a proximal end of the proximal collar to push against the distal end of the first tube.

2. The clot capturing assembly according to claim 1, wherein the proximal collar is attached to a distal end of the first tube.

3. The clot capturing assembly according to claim 1, wherein the proximal collar is not attached to a distal end of the first tube.

4. The clot capturing assembly according to claim 1, wherein the first retrieval device is configured to transition towards or to a fully expanded state at least partially by withdrawing the elongate wire proximally while the first tube is held stationary.

5. The clot retrieval assembly according to claim 1, wherein the distal collar is attached to the elongate wire.

6. The clot retrieval assembly according to claim 1, wherein the distal collar is not attached to the elongate wire.

7. The clot retrieval assembly according to claim 6, further comprising a stop on the distal end or distal end portion of the elongate wire, the stop including a proximal facing surface that faces a distal facing surface of the distal collar, the proximal facing surface of the stop being configured to press against the distal facing surface of the distal collar upon the elongate wire being withdrawn proximally to cause or assist in causing the first retrieval device to transition to or towards the fully expanded state.

8. The clot retrieval assembly according to claim 1, wherein the proximal collar, distal collar and the plurality of elongate clot capturing elements are made from a single piece of material.

9. The clot retrieval assembly according to claim 8, wherein the plurality of elongate clot capturing elements are laser cut from a tubular member.

10. The clot retrieval assembly according to claim 1, wherein one or more of the plurality of elongate clot capturing elements has a varying width and/or a varying thickness.

11. The clot retrieval assembly according to claim 1, wherein the distal end section of one or more of the plurality of elongate clot capturing elements has a cross-sectional area greater than the cross-sectional area of the respective one or more proximal end section of the one or more of the plurality of elongate clot capturing elements.

12. The clot retrieval assembly according to claim 1, wherein the distal end section of one or more of the plurality of elongate clot capturing elements has a thickness greater than a thickness of the respective one or more proximal end section of the one or more of the plurality of elongate clot capturing elements.

13. The clot retrieval assembly according to claim 1, wherein the distal end section of one or more of the plurality of elongate clot capturing elements has a width greater than a width of the respective one or more proximal end section of the one or more of the plurality of elongate clot capturing elements.

14. The clot retrieval assembly according to claim 4, further comprising an actuation device having a moveable first part to which a proximal end or proximal end section of the elongate wire is coupled, the actuation device having a stationary second part to which a proximal end or a proximal end section of the first tube is coupled.

15. The clot retrieval assembly according to claim 8, wherein the proximal end section of each of the plurality of elongate clot capturing elements is arranged helically with respect to a longitudinal axis of the first retrieval device.

16. The clot retrieval assembly according to claim 1, wherein the plurality of elongate clot capturing elements are configured to self-expand to place the first retrieval device in at least a partially expanded state.

17. The clot retrieval assembly according to claim 1, wherein none of the plurality of elongate clot capturing elements overlap with one another when the first retrieval device is in the unexpanded state.

18. The clot retrieval assembly according to claim 17, wherein at least some of the plurality of elongate clot capturing elements overlap with one another when the first retrieval device is in the fully expanded state.

19. The clot retrieval assembly according to claim 1, further comprising:
a second retrieval device mounted on the elongate wire at a location proximal to the first retrieval device, the second retrieval device comprising:
a distal collar mounted on the elongate wire, the distal collar of the second retrieval device having a distal facing surface;
a proximal collar mounted on the elongate wire and having a longitudinal through opening through which the elongate wire passes, the proximal collar of the second retrieval device having a proximal facing surface;
a plurality of elongate clot capturing elements that each has a proximal end attached to the proximal collar of the second retrieval device and a distal end attached to the distal collar of the second retrieval device, each of the plurality of elongate capturing elements of the second retrieval device having a proximal end portion and a distal end portion, each pair of neighboring elongate clot capturing elements of the second retrieval device being circumferentially separated from one another by an elongate slot that extends continuously between the proximal and distal collars of the second retrieval devices;
the second retrieval device being configured to transition from an unexpanded state to the fully expanded state, in the unexpanded state no portion of the plurality of elongate clot capturing elements of the second retrieval device is located over the proximal collar, in the fully expanded state at least some portions of the plurality of elongate clot capturing elements of the second retrieval device are inverted and residing over the proximal collar;
the first tube mounted on the elongate wire between the proximal collar of the first retrieval device and the distal collar of the second retrieval device, the first tube having a proximal facing surface that faces the distal facing surface of the distal collar of the second retrieval device; and
a second tube mounted on the elongate wire and having a longitudinal through opening through which the elongate wire passes, the proximal collar of the second retrieval device being attached to a distal end of the second tube or otherwise arranged such that the proximal facing surface of the proximal collar of the second retrieval device faces a distal facing surface of the second tube without the proximal collar being attached to the second tube;
the first and second retrieval devices being configured to transition from their respective unexpanded states towards or to their respective fully expanded states at least partially by withdrawing the elongate wire proximally while the second tube is held stationary.

20. The clot retrieval assembly according to claim 1, further comprising:
a second retrieval device comprising:
a distal collar mounted on the elongate wire, the distal collar having a distal facing surface;
a proximal collar mounted on the elongate wire and having a longitudinal through opening through which the elongate wire passes, the proximal collar having a proximal facing surface;
a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
the second retrieval device being configured to transition from an unexpanded state to an expanded state, in the unexpanded state no portion of the plurality of elongate clot capturing elements is located over the proximal collar, in the expanded state at least some portions of the plurality of elongate clot capturing elements are inverted and residing over the proximal collar;
a third retrieval device comprising:
a distal collar mounted on the elongate wire, the distal collar having a distal facing surface;
a proximal collar mounted on the elongate wire and having a longitudinal through opening through which the elongate wire passes, the proximal collar having a proximal facing surface;
a plurality of elongate clot capturing elements that each has a proximal end attached to the proximal collar and a distal end attached to the distal collar, each of the plurality of elongate capturing elements having a proximal end section and a distal end section, each pair of neighboring elongate clot capturing elements being circumferentially separated from one another by an elongate slot that extends continuously between the proximal and distal collars;
the third retrieval device being configured to transition from an unexpanded state to an expanded state, in the unexpanded state no portion of the plurality of elongate clot capturing elements is located over the proximal collar, in the expanded state at least some portions of the plurality of elongate clot capturing elements are inverted and residing over the proximal collar;
the first tube being located between the proximal collar of the first retrieval device and the distal collar of the second retrieval device, the first tube having a distal facing surface that faces the proximal facing surface of the proximal collar of the first retrieval device, the first tube having a proximal facing surface that faces the distal facing surface of the distal collar of the second retrieval device; and
a second tube mounted on the elongate wire and having a longitudinal through opening through which the elongate wire passes, the second tube being located between the proximal collar of the second retrieval device and the distal collar of the third retrieval device, the second tube having a distal facing surface that faces the proximal facing surface of the proximal collar of the second retrieval device, the second tube having a proximal facing surface that faces the distal facing surface of the distal collar of the third retrieval device;
a third tube mounted on the elongate wire and having a longitudinal through opening through which the elongate wire passes, the proximal collar of the third retrieval device being attached to a distal end of the third tube or otherwise arranged such that the proximal facing surface of the proximal collar of the third retrieval device faces a distal facing surface of the third tube without the proximal collar of the third retrieval device being attached to the third tube;
the first, second and third retrieval devices being configured to transition from their respective unexpanded states to their respective expanded states at least partially by withdrawing the elongate wire proximally while holding the third tube stationary.
